(19) United States
(12) Patent Application Publication
(10) Pub. No.: US 2023/0113802 A1
(43) Pub. Date: Apr. 13, 2023

(54) COMPOSITION AND METHOD TO PREPARE LONG-ACTING INJECTABLE SUSPENSION CONTAINING MULTIPLE CANCER DRUGS

(71) Applicant: University of Washington, Seattle, WA (US)

(72) Inventors: Rodney J.Y. Ho, Seattle, WA (US); James Griffin, Seattle, WA (US); Qingxin Mu, Seattle, WA (US); Yan Wu, Seattle, WA (US); Jesse Yu, Seattle, WA (US)

(73) Assignee: University of Washington, Seattle, WA (US)

(21) Appl. No.: 17/904,907

(22) PCT Filed: Feb. 25, 2021
(86) PCT No.: PCT/US2021/019726

§ 371 Date: Aug. 24, 2022

Related U.S. Application Data
(60) Provisional application No. 62/982,557, filed on Feb. 27, 2020.

(51) Int. Cl. A61K 31/7068; A61K 9/00; A61K 31/337; A61K 31/635; A61K 31/519; A61K 47/24; A61P 35/00; A61P 35/04

(52) U.S. Cl. CPC A61K 31/7068; A61K 9/0019; A61K 31/337; A61K 31/635; A61K 31/519; A61K 47/24; A61P 35/00; A61P 35/04

(57) ABSTRACT

The present disclosure describes an injectable aqueous dispersion, including an aqueous solvent, and a chemotherapeutic agent composition dispersed in the aqueous solvent to provide the injectable aqueous dispersion. The chemotherapeutic agent composition includes a combination of chemotherapeutic agents selected from: gemcitabine and paclitaxel; and venetoclax and zanubrutinib. The chemotherapeutic agent composition further includes one or more compatibilizers comprising a lipid (e.g., a lipid excipient), a lipid conjugate, or a combination thereof. The chemotherapeutic agents of the chemotherapeutic agent composition exhibit a synergistic chemotherapeutic effect.

### CROSS-REFERENCE TO RELATED APPLICATION

This application claims the benefit of U.S. Patent Application No. 62/982,557, filed Feb. 27, 2020, the disclosure of which is incorporated herein by reference in its entirety.

### STATEMENT OF GOVERNMENT LICENSE RIGHTS

This invention was made with government support under Grant No. UM1 AI120176, Grant No. R61 AI149665, and Grant No. U01 AI148055, awarded by the National Institutes of Health. The government has certain rights in the invention.

### BACKGROUND

Breast cancer is a leading cause of death in women in the U.S. and worldwide. Estimates suggest that in 2019 over 270,000 people were newly diagnosed and that 42,000 people would die of the disease in the US alone. A cure for breast cancer remains elusive. Early diagnosis, resection of breast cancer nodules, and receptor-targeted therapeutics (that inhibit human epidermal growth factor and hormone receptors) are effective at extending survival rates. However, many cancers still progress to the metastatic stage due to drug resistance and genetic mutation/evolution. Treatment options for these metastatic breast cancer patients are limited and outcomes are dismal. Even with current best agents— including drug-combinations and multiple cycle chemotherapy—treatments provide about 27% five-year survival. Patients at the metastatic stage exhibit cancer cells spread to highly perfused organs and local lymph nodes, detectable as colonies and nodules. Physiological mechanisms and the time-course of cancer cells metastasizing into lymph nodes and tissues are not fully understood. This gap has prevented the development of treatment interventions, specifically those targeted to these sites early in the course of advancing cancer.

Two recent reports from separate laboratories have provided time-and-spatial insight into the metastatic spread of breast cancer cells from primary sites (nodes and mammary gland) into blood (becoming apparent in the lungs as nodules) (Brown et al., Pereira et al., Science, 359, 2018). The two independent studies using 4T1 metastatic mouse tumors as models suggest that either removal of primary tumor or introducing a small number of cells in lymph node cortex (within the sinuses) would invariably lead to their appearance (through invasion into blood) in the lungs as nodules or colonies of breast cancer cells. The studies suggest that cancer cells rapidly proliferate in blood and migrate into the lungs to form colonies detectable as nodules. The time-course and spatial 4T1 tumor spread data thus suggests that early systemic intervention with highly-active chemotherapeutic or targeted agents responsive to metastatic cells could enhance response rate in metastatic breast cancer and delay the rate of disease progression.

According to NCCN (National Comprehensive Cancer Network) guidelines in the U.S., patients with newly diagnosed or recurrent breast cancer are treated with surgery if applicable prior to multiple cycles of adjuvant therapy. Metastatic breast cancer patients are often treated with intensive chemotherapeutic drug combinations targeted to topoisomerase or DNA synthesis plus microtubules, such as doxorubicin and paclitaxel, gemcitabine and paclitaxel (GT), capecitabine and docetaxel, or capecitabine and ixabepilone. These combination chemotherapies, while more effective than monotherapies, often exhibit dose-limiting toxicities, and intolerabilities prevent patients from completing their treatment cycles. For example, gemcitabine (1250 mg/m² IV day 1, day 8) and paclitaxel (175 mg/m² IV dl) combinations are reported to provide 41.4% response rate compared to paclitaxel alone (26.2%). Median survival of this combination as a first-line treatment was 18.6 months versus 15.8 months on paclitaxel only. In another study in patients who failed neo-adjuvant anthracycline-based chemotherapy, the same dose regimen produces a 50% objective response rate in the 12-month study. However, significant side effects such as neutropenia, leukopenia, and poor tolerability were reported for these combination therapies.

Drug combination regimens for treating cancer (e.g., metastatic breast cancer) are prescribed as a combination of two or more chemotherapeutics to maximize cancer cell death and overcome drug resistance. These regimens are typically based on anthracyclines (e.g., doxorubicin, daunorubicin, epirubicin) or taxanes (e.g., paclitaxel, docetaxel) in combination with other agents. Neither taxanes nor anthracyclines are superior to one another, but metastatic patients will likely have a limited duration of treatment with anthracyclines due to the cumulative lifetime risk of cardiac toxicity. This cumulative cardiotoxicity risk is inherent to anthracycline therapy. Once patients have reached their lifetime anthracycline dose, they can no longer be treated with doxorubicin, daunorubicin or epirubicin without the risk of heart failure. To overcome anthracycline dependent dose-limiting cardiotoxicity, taxane combinations with gemcitabine are used. However, GT is given as sequential infusions (T over 3 hours followed by G over 30 to 60 minutes) to minimize adverse events, which also reduces the time where both G and T circulate in plasma at pharmacologically relevant concentrations.

When G is given as a single agent, it requires intracellular phosphorylation to a tri-phosphate form to mediate cytotoxicity. In patients with leukemia, the concentration of G tri-phosphate in cancer cells is proportional to the plasma concentration of G up to 3 μg/mL. At higher plasma concentrations of G, the tri-phosphate levels no longer increase above 3 μg/mL; thus, this target concentration is currently used for G in plasma. The target therapeutic plasma concentrations of T were determined by establishing the threshold concentrations for neutropenia (0.09 μg/mL) with the intent to maximize T dosing before adverse events occur. Despite having target therapeutic plasma concentrations, there is only a 2-hour window in which G and T circulate above those concentrations under the current recommended dose and time sequences. This is because of the varying physicochemical and pharmacokinetic profile of GT. Longer or simultaneous infusions have been attempted in clinic, but poor patient tolerability and the physical incompatibility of GT limit these approaches.

To achieve the synchronized delivery of GT to target cells, drug delivery systems can be used carry multiple chemotherapeutic agents as a single particle. However, water soluble G (log P=−1.4) and water insoluble T (log P=3) are difficult to co-formulate with existing formulation strategies. Drug delivery systems such as liposomes (100 nm to several microns in diameter) or small polymeric nanoparticles (<10 nm in diameter) may, in some cases, mitigate systemic toxicity by reducing the high concentration of free drugs that cause toxicity. However, targeting these particles to cancer cells is a challenge. Biological barriers such as the reticuloendothelial system can sequester liposomes (>200 nm) into the liver and spleen for elimination. Thus, premature clearance prevents liposomal drugs from reaching target cells. Small polymeric nanoparticles or micelles (<10 nm) can undergo renal filtration and elimination by the kidney, leading to short plasma half-life and limited effect.

Challenges in chemotherapy and drug delivery are also seen in treatment of liquid tumors, such as the treatment of Chronic Lymphocytic Lymphoma (CLL). CLL is responsible for over one third of all new leukemia cases diagnosed every year, occurring in every 5 of 100,000 people. CLL is caused by the uncontrolled and monoclonal growth of malignant B cells. Broad-acting anticancer drugs, including chlorambucil (alkylating agent), fludarabine (purine analogue), and cyclophosphamide (alkylating agent), were effectively used to treat CLL prior to the introduction of newer targeted agents, though they each carry significant negative side effects that can limit their application in weaker and older patients. In addition, these drugs are unable to penetrate peripheral body compartments, preventing them from fully eliminating cancer cells in the body. Although conventional treatments for CLL were only able to treat and not cure the disease, new classes of small molecule and antibody drugs can target and eliminate malignant cells throughout the body, including in the lymphatic systems and other peripheral body compartments that were previously inaccessible to conventional treatments.

The most common targeted agents used in modern CLL treatment can be divided into three groups: (1) targeted kinase inhibitors (TKI's) of Bruton's Tyrosine Kinase (BTK), a kinase found in B cells, (2) inhibitors of Bcl-2, a mitochondrial antiapoptotic protein in B cells, and (3) monoclonal antibodies targeting CD20, an antigen present on B cell surfaces. All three groups of targeting agents selectively target B cells, both increasing their potency against CLL and reducing their off-target toxicities compared to conventional broad-acting therapies, making them the superior option when selecting treatments for a wide range of patients with CLL. Resistance events to targeted agents are not uncommon, especially when given as a monotherapy, so combination regimens of multiple drugs with varying mechanisms of action are often utilized. Combination regimens can also benefit the patients due to synergy between the regimen drugs: targeting multiple pathways can both limit resistance events and increase the potency of the treatment.

Ibrutinib, the first-generation small molecule TKI of BTK, is commonly used due to its effectiveness in treating CLL. Second-generation inhibitors of BTK, including acalabrutinib and zanubrutinib, are slowly being introduced into the market. BTK inhibitors are usually administered orally, making them an attractive treatment for patients. Combining ibrutinib with rituximab, an antibody agent targeting CD20, did not demonstrate improvement in response or progression-free survival in older patients, though some positive effect was seen in younger patients. Despite this disappointing outcome, combination regimens of ibrutinib and venetoclax, an inhibitor of Bcl-2, have shown promise against CLL in Phase II trials as a first-line treatment and as a second-line treatment for patients with relapsed or refractory CLL.

Venetoclax and zanubrutinib are administered orally, a route that patients usually prefer over parenteral routes, though the oral route can limit a drug's efficacy against disease. Gastrointestinal (GI) absorption of the drugs can be restricted due to metabolic enzymes in the gut and liver, leading to a low drug bioavailability, sub-therapeutic drug plasma and intracellular concentrations, and the subsequent promotion of drug resistance due to insufficient drug concentrations at the cancer site. In addition, orally delivered drug requires daily dosing, which can be cumbersome for the patient and leads to gastrointestinal injury due to constant high drug levels in the GI tract.

There is a need for effective chemotherapeutic drug combinations that can be delivered to advancing metastatic breast cancer cells or to liquid tumors, and that can be administered at a lower overall dose to overcome dose-limiting toxicities. The present disclosure fulfills these needs and provides further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features an injectable aqueous dispersion, including an aqueous solvent, and a chemotherapeutic agent composition dispersed in the aqueous solvent to provide the injectable aqueous dispersion. The chemotherapeutic agent composition includes a combination of chemotherapeutic agents selected from: gemcitabine and paclitaxel; and venetoclax and zanubrutinib; the chemotherapeutic agent composition further includes one or more compatibilizers that includes a lipid (e.g., a lipid excipient), a lipid conjugate, or a combination thereof. The chemotherapeutic agents of the chemotherapeutic agent composition exhibit a synergistic chemotherapeutic effect.

In yet another aspect, the present disclosure features a method of treating cancer, including parenterally administering to a subject in need thereof an injectable aqueous dispersion described herein, wherein the chemotherapeutic agents of the chemotherapeutic agent composition exhibit a synergistic chemotherapeutic effect.

In yet a further aspect, the present disclosure features a powder composition including a combination of chemotherapeutic agents selected from: gemcitabine and paclitaxel; and venetoclax and zanubrutinib. The powder composition further includes one or more compatibilizers including a lipid (e.g., a lipid excipient), a lipid conjugate, or a combination thereof. The chemotherapeutic agents of the combination of chemotherapeutic agents exhibit a synergistic chemotherapeutic effect.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIGS. 1A-1D are directed to the effect of drug combination nanoparticle (DcNP) on gemcitabine and paclitaxel fixed-dose combination treatment on 4T1 metastatic tumor intensity and nodules in the lungs. Mice inoculated with 4T1-luc via tail vein were administered with a 50/5 mg/kg GT fixed dose combination in DcNP (test) or CrEL (control) formulation as a single bolus IV dose. On day 14, the total tumor growth was estimated. Equivalent single IV doses of conventional formulation of the GT fixed dose combination were given to mice as a control. The values in panels (A) and (B) are expressed as mean±SEM. P values were obtained from two-tailed t-tests with unequal variations. Experimental animal numbers in each group were 8-15.

FIG. 1A is a bar graph of the total tumor growth on day 14 based on luciferase activity detected as total bioluminescence (BL) intensity.

FIG. 1B is a bar graph of the total tumor growth on day 14 based on the cancer nodule count.

FIG. 1C is a series of photographs of Representative 4T1-luc luciferase mediated bioluminescence intensities in saline control, CrEL drug combination, DcNP treated mice, and healthy mice, as well as the lung nodules harvested from these mice.

FIG. 1D is a series of images of GFP (expressed by 4T1-luc) stained lung cross-sections from mice in conditions of (C), and photographs of fixed lung tissues. Top row, whole lung cross-sections; Bottom row, enlarged images from red boxes in top row. Black arrows indicate cancer nodules.

FIG. 2A-2B are directed to the dose-response of DcNP formulated gemcitabine-paclitaxel on inhibiting 4T1 lung metastasis; and bodyweight reduction. The 4T1-luc breast cancer cells were inoculated via tail-vein and the indicated dose (anchored on gemcitabine containing 1/10 weight equivalent of paclitaxel in DcNP formulation) were administered as a single dose IV administration. The 4T1 tumor growth (based on 4T1-luc luciferase dependent bioluminescence) and tumor nodule counts were expressed as therapeutic effects. The bodyweight loss at day 4 was used as an indicator of gross toxicity. FIG. 2A is a series of photographs of representative 4T1-luc luciferase mediated bioluminescence intensities in saline and DcNP (with different GT doses) treated mice, as well as the lung nodules harvested from these mice.

FIG. 2B is a graph of dose-responsive curves of metastasis inhibition determined by bioluminescence integration and nodule count, as well as body weight loss with DcNP treatment. The values expressed are mean±SEM. Experimental animal numbers in each group were 8-15. The curves were fitted in GraphPad Prism software (dose response-inhibition) to estimate ED₅₀s and TD₅₀s based on gemcitabine doses. The ED₅₀ was averaged from two measures. The average therapeutic index (TI) is estimated based on the ratio of TD₅₀-to-ED₅₀ which is 15.8.

FIG. 3 is a graph of time course body weight changes in 4T1-inoculated mice treated with placebo (saline), GT in Cremophor EL/EtOH/PBS (CrEL) suspension or DcNP (drug-combination nanoparticle) dosage form. On day 0 GT in CrEL suspension or DcNP at 1.25/0.125, 10/1, or 50/5 mg/kg IV doses, and the 4T1 inoculated mice were monitored over 14 days. Each treatment group contains 8-15 mice and the data presented are mean±SEM. In the group of 50/5 mg/kg of DcNP treated mice, some animals, due to clinical necessity, were sacrificed ahead of schedule. The remaining animals in the high dose group recovered and by day 14 appeared to exhibit body weight higher than at entry. In comparison, the saline placebo treated animals exhibit significant (15%) weight loss by day 14 due to rapid growth of lung metastatic nodules. The same trend is seen in the group treated with GT in CrEL suspensions (at two lower doses-10/1 and 1.25/0.125 mg/kg).

FIG. 4 is a schematic representation of a mechanism-based pharmacokinetic model for DcNP associated and dissociated gemcitabine and paclitaxel in plasma after IV dosing. A mechanism-based pharmacokinetic (MBPK) model was developed to describe the association and dissociation of drug from DcNPs in plasma. The model features two parts: (A) the behavior of the fraction of gemcitabine or paclitaxel associated to DcNPs and their distribution to peripheral compartments. (B) The behavior of the fraction of DcNP dissociated gemcitabine or paclitaxel in plasma including distribution into peripheral compartments and clearance as dissociated drug. The DcNP associated and dissociated models are linked by the release parameter k_{1,3} in the central compartment. After dissociation through the release parameter, gemcitabine and paclitaxel are assumed to behave as they would in their free drug form as presented in the conventional CrEL dosage form control.

FIGS. 5A and 5B are directed to the structural morphology of GT DcNPs by electron microscopy. The morphology of GT DcNPs was evaluated using negatively stained transmission election microscopy and compared against conventional liposomes.

FIG. 5A is an electron micrograph of GT DcNPs, which exhibit a discoid morphology with no evidence of bilayer structure (dashed arrows).

FIG. 5B is a comparison electron micrograph of conventional liposome controls, which exhibit typical spherical structures with visible bilayer membranes (solid arrows).

FIGS. 6A and 6B demonstrate that the association of GT to DcNPs increases the concentration of GT in plasma over time compared to CrEl control suspension.

FIG. 6A is a graph showing gemcitabine (50 mg/kg) administered as a DcNP (Dashed, ∘) substantially increases the plasma circulation levels in healthy BALB/c mice (n=3 per time point) measured at identical time points to the CrEL control (Cremophor El/saline suspension, solid line, •). The LLOQ of gemcitabine is represented as a dotted line

FIG. 6B is a graph showing that the plasma concentration of paclitaxel (5 mg/kg, Panel B) was also increased in plasma relative to the control suspension but a smaller effect is observed. Paclitaxel levels fall below the LLOQ of our LC-MS/MS assay after 6 hours. The LLOQ of paclitaxel is represented as a dotted line.

FIGS. 7A-7C are a series of graphs directed to the effect of DcNP formulation on dFdU formation over time compared to CrEL control.

FIG. 7A is a graph showing the plasma time course of gemcitabine (A) and its metabolite dFdU (▴) in control soluble gemcitabine (50 mg/kg; in CrEL) dosage form.

FIG. 7B is a graph showing the plasma time course of mice treated with gemcitabine in GT DcNP at equivalent doses to the soluble control; the symbols are the same as those represented in FIG. 7A.

FIG. 7C is a graph showing the ratios of gemcitabine to dFdU over time for mice treated with gemcitabine, comparing gemcitabine in a DcNP (∘) or CrEL (•) control dosage form.

FIGS. 8A and 8B are a series of graphs showing the validation of an MBPK model predicted concentration time curve for gemcitabine and paclitaxel with experimental data in mouse plasma after intravenous administration of GT DcNPs.

FIG. 8A is a graph showing the gemcitabine plasma time course of associated and dissociated fractions of drug. The experimental data are presented in open circles (∘) with an SD error bar. The MBPK model simulated values are plotted as a continuous solid line. The dotted lines represent the DcNP dissociated gemcitabine concentration over time, simulated by the model.

FIG. 8B is a graph showing the experimental data and simulated DcNP associated and dissociated fractions over time for paclitaxel. The symbol and line representations for FIG. 8B are the same as for FIG. 8A. The total simulated plasma concentrations and the DcNP associated species of gemcitabine overlap with most of the gemcitabine remaining DcNP associated throughout the study period.

FIGS. 9A and 9B are bar graphs showing the effects of DcNP on gemcitabine and paclitaxel tissue distribution 3 hours after intravenous injection compared to the control suspension. Mice (n=3) were intravenously administered with GT DcNP or a control dosage form (CrEL suspension) at 50 mg/kg gemcitabine and 5 mg/kg paclitaxel. Gemcitabine and paclitaxel concentrations were measured in the listed tissues 3 hours after injection; the respective tissue to plasma ratios for each animal were analyzed and presented as a mean±SD for each dosage form.

FIG. 9A is a graph of gemcitabine tissue to plasma ratio. The black bars indicate GT DcNP while the gray bars indicate the CrEL control dosage form. *denotes p <.05.

FIG. 9B is a graph of paclitaxel tissue to plasma ratios. The black bars indicate GT DcNP while the gray bars indicate the CrEL control dosage form. *denotes p<0.05.

FIG. 10 is a table describing the particle size determination of DcNPs. Particle size and distribution of different DcNP formulations (with and without TWEEN20) at Day 1 and Day 70 following rehydration without sonication. DcNP's are initially in solution at Day 1, but naturally precipitate over time, as seen at Day 70. “Supernatant” refers to particles in solution following precipitation, while the “mixture” refers to the fully mixed DcNP suspension.

FIG. 11 is a table describing the association efficiency of venetoclax and zanubrutinib in DcNP's. Particle size and distribution of different DcNP formulations (with and without TWEEN20) at Day 1 and Day 70 following rehydration without sonication. DcNP's are initially in solution at Day 1, but naturally precipitate over time, as seen at Day 70. “Supernatant” refers to particles in solution following precipitation, while the “mixture” refers to the fully mixed DcNP suspension.

FIGS. 12A-12D are a series of graphs of the in vitro effect of free drug and drug combination nanoparticles on cell growth.

FIG. 12A is a graph of HL-60 viability as a function of free venetoclax.

FIG. 12B is a graph of HL-60 viability as a function of free zanubrutinib.

FIG. 12C is a graph of HL-60 viability as a function of a combination of free venetoclax and zanubrutinib.

FIG. 12D is a graph of HL-60 viability as a function of a DcNP including venetoclax and zanubrutinib.

FIGS. 13A-13D are a series of graphs showing the intracellular drug concentrations following incubation with free or DcNP-bound drug. Three leukemic cell lines were incubated with either free or DcNP-bound drug over four hours, and intracellular drug concentration was measured via LC-MS/MS. Both free drugs show relatively little drug uptake compared to the DcNP formulation. Free drug concentrations are roughly a quarter of the DcNP drug concentrations.

FIG. 13A is a graph of intracellular uptake of free venetoclax.

FIG. 13B is a graph of intracellular uptake of DcNP-bound venetoclax.

FIG. 13C is a graph of intracellular uptake of free zanubrutinib.

FIG. 13D is a graph of intracellular uptake of DcNP-bound zanubrutinib.

FIGS. 14A-14D are directed to the ABT-199 and BGB-3111 pharmacokinetics in Mice. Following intravenous (IV) or subcutaneous (SC) administration of venetoclax and zanubrutinib, plasma drug concentrations were measured over one week. Data points below the limit of quantification were not plotted. Subcutaneous administration of DcNP's yielded the largest drug exposure for both drugs compared to intravenous administration of either free drug or DcNP's.

FIG. 14A is a graph of venetoclax (ABT-199) pharmacokinetics in mice.

FIG. 14B is a graph of zanubrutinib (BGB-3111) pharmacokinetics in mice.

FIG. 14C is a table of AUC values of intravenously or subcutaneously administered therapeutic agents.

FIG. 14D is a table of AUC ratios of intravenously or subcutaneously administered therapeutic agents.

### DETAILED DESCRIPTION

The present disclosure describes an injectable aqueous dispersion, including an aqueous solvent, and a chemotherapeutic agent composition dispersed in the aqueous solvent to provide the injectable aqueous dispersion. The chemotherapeutic agent composition includes a combination of chemotherapeutic agents selected from: gemcitabine and paclitaxel; and venetoclax and zanubrutinib. The chemotherapeutic agent composition further includes one or more compatibilizers comprising a lipid (e.g., a lipid excipient), a lipid conjugate, or a combination thereof. The chemotherapeutic agents of the chemotherapeutic agent composition exhibit a synergistic chemotherapeutic effect, such that when administered together, the therapeutic effect of the composition is greater than the added therapeutic effect of each of the individual therapeutic agent when administered in free form, and/or compared to the combination of the chemotherapeutic agents when administered together in an amorphous form.

The chemotherapeutic agent composition is simple, stable, and scalable; and can be in the form of a drug combination nanoparticle (DcNP). The composition can provide chemotherapeutic therapeutic agents in long-acting injectable forms that provide a low, effective, and sustained dose for chemotherapy. A mixture of water-soluble and water-insoluble chemotherapeutic agents, which are generally incompatible and cannot be formed into a single unified composition, can be formulated together to provide long-acting injectable dosage forms, which exhibit sustained plasma levels for all the chemotherapeutic agents in the composition.

Without wishing to be bound by theory, it is believed that the stable assembly of otherwise incompatible water-soluble and water-insoluble chemotherapeutic agents is facilitated by lipid excipients through a well-defined formulation process. In some embodiments, the chemotherapeutic agents differ in water-solubility, such that the chemotherapeutic agents in a given composition can be water-insoluble, but differ in water solubility on the order of greater than 1, 2, or 3 orders or magnitude or more, and the lipid excipients can still facilitate the stable assembly of the chemotherapeutic agents through a well-defined formulation process. The unique drug-combination platform technology, called a drug combination nanoparticle (DcNP), could stabilize water-insoluble and water-soluble chemotherapeutic drugs, or chemotherapeutic drugs having very different water-solubilities in an injectable long-acting suspension that provides sustained and synergistic therapeutic effects.

### Definitions

At various places in the present specification, groups or ranges are described. It is specifically intended that the disclosure include each and every individual sub-combination of the members of such groups and ranges.

The verb “comprise” and its conjugations, are used in the open and non-limiting sense to mean that items following the word are included, but items not specifically mentioned are not excluded.

“About” in reference to a numerical value refers to the range of values somewhat less or greater than the stated value, as understood by one of skill in the art. For example, the term “about” could mean a value ranging from plus or minus a percentage (e.g., ±1%, ±2%, or 5%) of the stated value. Furthermore, since all numbers, values, and expressions referring to quantities used herein are subject to the various uncertainties of measurement encountered in the art, unless otherwise indicated, all presented values may be understood as modified by the term “about.”

As used herein, the articles “a,” “an,” and “the” may include plural referents unless otherwise expressly limited to one-referent, or if it would be obvious to a skilled artisan from the context of the sentence that the article referred to a singular referent.

Where a numerical range is disclosed herein, such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include 1 and 10, and any and all subranges between the minimum value of 1 and the maximum value of 10. Exemplary subranges of the range “1 to 10” include, but are not limited to, e.g., 1 to 6.1, 3.5 to 7.8, and 5.5 to 10.

As used herein, the term “matrix” denotes a solid mixture composed of a continuous phase, and one or more dispersed phase(s) (e.g., particles of the pharmaceutically active agent).

The terms “therapeutic agent”, “active agent”, “drug”, and “active pharmaceutical ingredient” are used interchangeably herein.

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety or a molecule that is not attracted to water with significant apolar surface area at physiological pH and/or salt conditions. This phase separation can be observed via a combination of dynamic light scattering and aqueous NMR measurements. A hydrophobic therapeutic agent has a log P value of 1 or greater.

As used herein, the term “hydrophilic” refers to a moiety or a molecule that is attracted to and tends to be dissolved by water. The hydrophilic moiety is miscible with an aqueous phase. A hydrophilic therapeutic agent has a log P value of less than 1.

The log P values of hydrophobic and hydrophilic drugs can be found, for example, at pubchem.ncbi.nlm.nih.gov and drugbank.ca.

As used herein, the log P value is a constant defined in the following manner:

Log P=log 10 (Partition Coefficient)

Partition Coefficient, P=[organic]/[aqueous]

where [ ] indicates the concentration of solute in the organic and aqueous partition. A negative value for log P means the compound has a higher affinity for the aqueous phase (it is more hydrophilic); when log P=0 the compound is equally partitioned between the lipid and aqueous phases; a positive value for log P denotes a higher concentration in the lipid phase (i.e., the compound is more lipophilic). Log P=1 means there is a 10:1 partitioning in organic: aqueous phases. The most commonly used lipid and aqueous system is octan-1-o1 and water, or octanol and buffer at a pH of 6.5 to 8.5.

As used herein, the term “water-insoluble” refers to a compound that has a water-solubility of less than 0.2 mg/mL (e.g., less than 0.1 mg/mL, or less than 0.01 mg/mL)), at a temperature of 25° C., and at a pressure of 1 atm or 101.3 kPa.

As used herein, the term “water-soluble” refers to a compound that is soluble in water in an amount of 1 mg/ml or more (e.g., 2 mg/ml or more), at a temperature of 25° C., and at a pressure of 1 atm or 101.3 kPa.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “polymer” refers to a macromolecule having more than 10 repeating units.

As used herein, the term “small molecule” refers to a low molecular weight (<2000 Daltons) organic compound that may help regulate a biological process, with a size on the order of 1 nm. Most drugs are small molecules.

A number of chemotherapeutic agents are referred to herein. Their names, molecular formula, molecular weight, water solubility, and structures are provided below.

Gemcitabine (G); also known as 2′, 2′-difluoro 2′deoxycytidine, and dFdC. Molecular formula: C₉H₁₁F₂N₃O₄. Molecular weight: 263.201 g/mol. Water solubility of 5.13×10⁴ mg/L at 25° C.; log P=−2.01. IUPAC Name: 4-amino-1-[(2R,4R,5R)-3,3-difluoro-4-hydroxy-5-(hydroxymethyl)oxolan-2-yl]pyrimidin-2-one. Chemical structure:

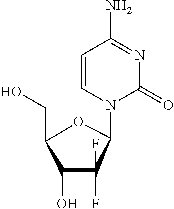

Paclitaxel (T); also known as Taxol. Molecular formula: C₄₇H₅₁NO₁₄. Molecular weight: 853.918 g/mol. Water solubility of 0.00556 mg/mL at 25° C.; log P=3.2. IUPAC Name: [(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0^{4,7}]heptadec-13-en-2-yl] benzoate. Chemical structure:

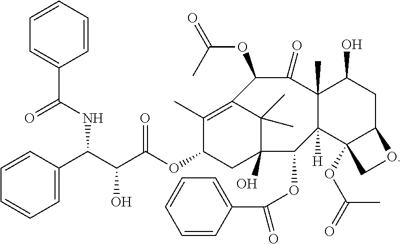 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Venetoclax; also known as Venclexta, Venclyxto, GDC-0199, ABT-199, and RG-7601. Molecular formula: C₄₅H₅₀ClN₇O₇S. Molecular weight: 868.45 g/mol. Water solubility of 0.000933 mg/mL at 25° C.; log P=6.92. IUPAC Name: 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide. Chemical structure:

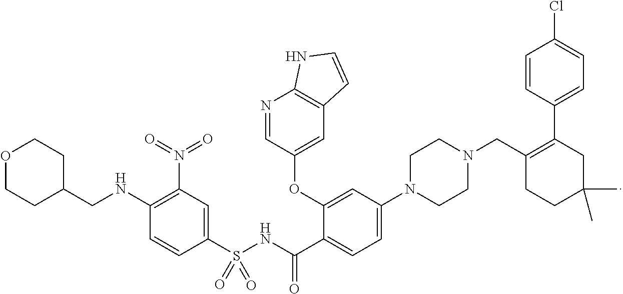 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Zanubrutinib; also known as Brukinsa, and BGB-3111. Molecular formula: C₂₇H₂₉N₅O₃. Molecular weight: 471.5509 g/mol. Water solubility of 0.0103 mg/mL at 25° C.; log P=3.5. IUPAC Name: (7S)-2-(4-phenoxyphenyl)-7-(1-prop-2-enoylpiperidin-4-yl)-4,5,6,7-tetrahydropyrazolo[1,5-a]pyrimidine-3-carboxamide. Chemical structure:

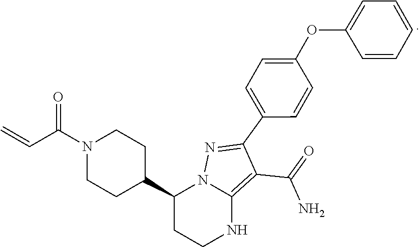 [SMILES: C=CC(=O)N1CCC([C@@H]2CCNc3c(C(N)=O)c(-c4ccc(Oc5ccccc5)cc4)nn32)CC1]

As used herein, “absorption profile” refers to the rate and extent of exposure of a drug/combination of drugs, data analysis of the AUC and/or Cₘₐₓ including the curves thereof.

As used herein, “freely solubilized individual therapeutic agent” or “free soluble therapeutic agent” refers to a single therapeutic agent, or a salt thereof, fully dissolved in a pharmaceutically acceptable solvent such as saline, a buffer, or dimethyl sulfoxide (DMSO) (for experimental studies but not approved for formulating injectable as a solvent), without excipients such as a lipid and/or a lipid conjugate.

As used herein, “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound/combination of compounds, as well as providing directions to carry out a method involving a particular compound/combination of compounds or a dosage form comprising the compound/combination of compounds.

As used herein, a “composition” refers to a collection of materials containing the specified components. One or more dosage forms may constitute a composition, so long as those dosage forms are associated and designed for use together.

As used herein, a “pharmaceutical composition” refers to a formulation of a compound/combination of compounds of the disclosure, and a medium generally accepted in the art for the delivery of the biologically active compound to mammals, e.g., humans. Such a medium includes all pharmaceutically acceptable carriers, diluents, or excipients therefor. The pharmaceutical composition may be in various dosage forms or contain one or more unit-dose formulations. The pharmaceutical composition can provide stability over the useful life of the composition, for example, for a period of several months. The period of stability can vary depending on the intended use of the composition.

As used herein, “salts” include derivatives of an active agent, wherein the active agent is modified by making acid or base addition salts thereof. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

As used herein, a “solid dispersion” relates to a solid system comprising a nearly homogeneous or homogeneous dispersion of an active ingredient/combination of active ingredients, in an inert carrier or matrix.

As used herein, a “homogeneous mixture” or “homogeneous distribution” refers to a mixture in which the components (e.g., APIs and excipients) are uniformly distributed throughout the mixture, which can be, for example, a suspension, a powder, or a solution. The mixture can have the same physical properties at every macroscopic sampling point of the assembled drug combination product.

As used herein, an “aqueous dispersion” refers to an aqueous suspension where the APIs and excipients of the pharmaceutical composition are suspended in a solvent or a buffer

“Prodrug” refers to a precursor of the pharmaceutically active agent wherein the precursor itself may or may not be pharmaceutically active but, upon administration, will be converted, either metabolically or otherwise, into the active agent or drug of interest. For example, prodrug includes an ester or an ether form of an active agent.

Particular pharmacokinetic parameters are defined in Table A.

TABLE A

| Parameter | Definition |
|---|---|
| AUC_{0-t last} | Area under the plasma concentration-time curve |
| | from time zero up to the last quantifiable concentration |
| AUC_{0-∞} | Area under the plasma concentration-time curve |
| | from time zero to infinity |
| % | Percentage of AUC that is due to extrapolation from |
| AUCₑₓₜᵣₐₚ | t last to infinity |
| Cₘₐₓ | Maximum observed plasma concentration |
| tₘₐₓ | Time of the maximum observed plasma concentration |
| t_{lag} | Time before the start of absorption |
| tₗₐₛₜ | Time of the last quantifiable plasma concentration |
| t_{1/2} | Apparent plasma terminal elimination half-life |
| | (terminal half-life) |

It is noted that AUC₀₋ₜ and AUC₀₋ₜₗₐₛₜ are used interchangeably herein. Also, AUC_{inf} and AUC_{t-inf} are used interchangeably with AUC_{0-∞}. It should also be understood that, unless otherwise specified, all pharmacokinetic parameters are measured after a single administration of the specified amount of a therapeutic agent/combination of therapeutic agents followed by a washout period in which no additional therapeutic agent/combination of therapeutic agents is administered.

A “terminal half-life” refers to the time required to divide the plasma concentration by two after reaching pseudo-equilibrium, and not the time required to eliminate half the administered dose. This is typically referred to as the last phase of descending plasma drug concentration over time and just before the drug is eliminated from the body.

A “therapeutically effective plasma concentration” refers to a plasma concentration of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition, or disorder; and

(3) ameliorating the disease; for example, ameliorating a disease, condition or disorder in an individual who is experiencing or displaying the pathology or symptomatology of the disease, condition, or disorder (i.e., reversing the pathology and/or symptomatology) such as decreasing the severity of disease.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition, or disorder; and

(3) ameliorating the disease; for example, ameliorating a disease, condition or disorder in an individual who is experiencing or displaying the pathology or symptomatology of the disease, condition, or disorder (i.e., reversing the pathology and/or symptomatology) such as decreasing the severity of disease.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

As used herein, the term “composite” refers to a composition material, a material made from two or more constituent materials with significantly different physical or chemical properties that, when combined, produce a material with characteristics different from the individual components. The individual components remain separate and distinct within the finished structure.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Chemotherapeutic Agent Formulations

Powder Chemotherapeutic Agent Compositions

The present disclosure features a powder composition including a combination of chemotherapeutic agents such as a combination of gemcitabine and paclitaxel; or a combination of venetoclax and zanubrutinib. The powder composition includes one or more compatibilizers such as a lipid (e.g., a lipid excipient), a lipid conjugate, or a combination thereof. The chemotherapeutic agents of the combination of chemotherapeutic agents exhibit a synergistic chemotherapeutic effect.

The chemotherapeutic agent compositions of the present disclosure can form a homogeneous powder (e.g., a lyophilized homogeneous powder) having a homogeneous distribution of each chemotherapeutic agent when viewed by scanning electron microscopy, such that each individual component is not visually discernible at 10-20 kV. The chemotherapeutic agent compositions have a unified repetitive multi-drug motif (MDM) structure (used interchangeably herein with “multi-drug-lipid motif” and “multi-drug motif”), such that, unlike amorphous powders, the chemotherapeutic agent compositions of the present disclosure have long range order, in the form of repetitive multi-drug and unified motifs. These motifs are homogenous or evenly distributed throughout the powder at any sampling point as determined by X-ray diffraction analysis, which can discern the physical organization of the drug combination structure stabilized by compatibilizer(s), which are homogenously distributed among the different therapeutic agent molecules.

The chemotherapeutic agent compositions (which, as discussed above, can be in the form of a powder) can be made by fully dissolving water-insoluble chemotherapeutic agents and one or more compatibilizers in an alcoholic solvent, dissolving water-soluble chemotherapeutic agents in water or a water-based aqueous buffer; adding the buffer solution to the alcoholic solution to provide a mixture (e.g., a fully solubilized homogenous therapeutic agent and compatibilizer together in solution state), followed by a controlled removal of solvent in a process (e.g., a defined and controlled process) that locks the chemotherapeutic agent and excipients into a unique powder product free of solvent and that has multi-drug motifs (MDM) with long range translational periodicity. In some embodiments, the water-insoluble chemotherapeutic agents, the one or more compatibilizers, and the water-soluble chemotherapeutic agents are dissolved in an alcoholic solvent (e.g., methanol, ethanol, and/or propanol) at a temperature of 60-80° C., then the solvent is removed in a defined and controlled process to lock the chemotherapeutic agent and excipients into a unique powder product free of solvent and that has multi-drug motifs (MDM) with long range translational periodicity. These motifs are structurally different from purely amorphous material as verified by powder x-ray diffraction, and the chemotherapeutic agent compositions can be hydrated and homogenized to produce long-acting injectable aqueous dispersions (e.g., in the form of a suspension) with the chemotherapeutic agents, having a stability in suspension when stored for over 12 months at 4° C. or at 25° C. The percentage of drug associated to the drug-combination particles is reproducible, and the particles are physically and chemically stable; thus, suitable for pharmaceutical preparation of long-acting injectable dosage form. The stable chemotherapeutic agent compositions can provide long-acting therapeutic combinations having extended plasma chemotherapeutic agent concentrations for the chemotherapeutic agent components, compared to separately administered individual free chemotherapeutic agent components, or an amorphous mixture of the chemotherapeutic agents and excipients.

The chemotherapeutic agent compositions can have a powder X-ray diffraction pattern that has at least one peak having a signal to noise ratio of greater than 3 (e.g., greater than 4, greater than 5, or greater than 6). The at least one peak can have a different 2θ peak position than the diffraction peak 2θ positions of each individual component (e.g., each individual therapeutic agent, or each individual therapeutic agent and excipient) of the chemotherapeutic agent compositions. The at least one peak can have a different 2θ peak position than the diffraction peak 2θ positions for a simple physical mixture of the individual components of the chemotherapeutic agent compositions. The X-ray diffraction pattern of the chemotherapeutic agent compositions are indicative of multiple chemotherapeutic agents assembled into a unified domain having repeating identical units, such that the chemotherapeutic agents and the one or more compatibilizers together form an organized composition (as seen by the discrete powder X-ray diffraction peaks, described above). The organized composition can have a long-range order in the form of a repeating pattern organized as one unified structure, distinctly different from each X-ray diffraction profile for the drugs and lipid excipients. As used herein, short range order involves length scales of from 1 Å (or 0.1 nm) to 10 Å (or 1 nm), while long-range order has length scales that exceed 10 nm, or of an order that is at 2 theta 10-25 nm. The long-range order can be a characteristic feature of molecular spacing for a given molecule. Thus, the chemotherapeutic agent compositions of the present disclosure have a unified repetitive multi-drug motif (MDM) structure and is referred to interchangeably herein as an “MDM composition.” MDM structures are described, for example, in Yu et al., J Pharm Sci 2020 November; 109(11):3480-3489, incorporated herein by reference in its entirety.

In some embodiments, the present disclosure features chemotherapeutic agent compositions that include a combination of chemotherapeutic agents selected from gemcitabine and paclitaxel; and venetoclax and zanubrutinib. The chemotherapeutic agent compositions include a mixture of water-soluble and water-insoluble chemotherapeutic agents.

In some embodiments, the combination of chemotherapeutic agents is gemcitabine: paclitaxel, in a molar ratio of from about 1:1 (e.g., from about 2:1, from about 5:1, from about 10:1, from about 20:1, from about 25:1, from about 30:1, from about 40:1, or from 45:1) to about 50:1 (e.g., to about 45:1, to about 40:1, to about 30:1, to about 25:1, to about 20:1, to about 10:1, to about 5:1, or to about 2:1). In certain embodiments, the combination of chemotherapeutic agents is venetoclax and zanubrutinib, in a molar ratio of from about 10:1 (e.g., from about 8:1, from about 6:1, from about 4:1, from about 2:1, from about 1:1, from about 1:2, from about 1:3, from about 1:5, from about 1:7, or from about 1:9) to about 1:10 (e.g., to about 1:9, to about 1:7, to about 1:5, to about 1:3, to about 1:2, to about 1:1, to about 2:1, to about 4:1, to about 6:1, to about 8:1).

In some embodiments, the chemotherapeutic agent compositions of the present disclosure exhibit a therapeutically effective plasma concentration of the combination of chemotherapeutic agents for 2 or more weeks (e.g., 3 or more weeks, 4 or more weeks 5 or more weeks, 6 or more weeks, 7 or more weeks, or 8 or more weeks), when administered to a subject in need thereof as a bolus dose.

The chemotherapeutic agent compositions of the present disclosure further include one or more compatibilizers such as a lipid and/or a lipid conjugate, in addition to the combination of chemotherapeutic agents. In some embodiments, the one or more compatibilizers is present in the chemotherapeutic agent composition in an amount of 60 wt % or more (e.g., 70 wt % or more, 80 wt % or more, 90 wt % or more) and 95 wt % or less (e.g., 90 wt % or less, 80 wt % or less, or 70 wt % or less) relative to the weight of the total chemotherapeutic agent composition. In some embodiments, the one or more compatibilizers, such as a covalent conjugate of a lipid with a hydrophilic moiety (e.g., PEG-DSPE, mPEG-DSPE, or mPEG₂₀₀₀-DSPE), is present in the chemotherapeutic agent composition in an amount of 2 mole % or more (e.g., 5 mole % or more, 8 mole % or more, or 10 mole % or more) and 15 mole % or less (e.g., 10 mole % or less, 8 mole % or less, or 5 mole % or less) relative to the total compatibilizer content. In some embodiments, the one or more compatibilizers, such as a covalent conjugate of a lipid with a hydrophilic moiety (e.g., PEG-DSPE, mPEG-DSPE, or mPEG₂₀₀₀-DSPE), is present in the chemotherapeutic agent composition in an amount of 10 mole % relative to the total compatibilizer content. In some embodiments, a covalent conjugate of a lipid with a hydrophilic moiety (e.g., PEG-DSPE, mPEG-DSPE, or mPEG₂₀₀₀-DSPE) in a mole percent of lower than 15% (e.g., 12%, or 10%) compared to the total compatibilizer content provides a composition exhibiting a sustained therapeutically effective plasma concentration of the constituent therapeutic agents over a period of at least 1 week (e.g., at least 2 weeks, at least 3 weeks, or at least 1 month), while a mole percent of greater than 15% (e.g., 20% or more) provides a therapeutically effective plasma concentration half-life of less than 2 days.

The one or more compatibilizers can include at least one lipid excipient and at least one lipid conjugate excipient. For example, the one or more compatibilizers can include at least one lipid excipient in an amount of 50 wt % or more and 80 wt % or less. The lipid excipient can be a saturated or unsaturated lipid excipient, such as a phospholipid. The phospholipid can include, for example, 1,2-distearoyl-sn-glycero-3-phosphocholine (DSPC), 1,2-dimyristoyl-sn-glycero-3-phosphocholine (DMPC), 1,2-dipalmitoyl-sn-glycero-3-phosphocholine (DPPC). In some embodiments, the one or more compatibilizers include at least one lipid conjugate excipient in an amount of 19 wt % or more and 25 wt % or less relative to the weight of the total chemotherapeutic agent composition. The lipid conjugate excipient can be a covalent conjugate of a lipid with a hydrophilic moiety. The hydrophilic moiety can include a hydrophilic polymer, such as poly(ethylene glycol) having a molecular weight (Mₙ) of from 500 to 5000 (e.g., from 500 to 4000, from 500 to 3000, from 500 to 2000, from 1000 to 5000, from 1000 to 4000, from 1000 to 3000, from 1000 to 2000, from 2000 to 5000, from 2000 to 4000, from 2000 to 3000, 2000, 1000, 5000, or 500). In some embodiments, the lipid conjugate excipient is a conjugate of 1,2-distearoyl-sn-glycero-3-phosphoethanolamine (DSPE) with PEG, such as PEG₂₀₀₀ or mPEG₂₀₀₀ The PEG can be conjugated to the lipid via an amide linkage. The lipid conjugate excipient can be in the form of a salt, such as an ammonium or a sodium salt.

In some embodiments, the one or more compatibilizers is 1,2-distearoyl-sn-glycero-3-phosphocholine and/or 1,2-distearoyl-sn-glycero-3-phosphoethanolamine-N-[poly(ethylene glycol)2000]. In some embodiments, the compatibilizers in the chemotherapeutic agent composition is 1,2-distearoyl-sn-glycero-3-phosphocholine and 1,2-distearoyl-sn-glycero-3-phosphoethanolamine-N-[poly(ethylene glycol)2000].

The chemotherapeutic agent compositions in powder form can include the chemotherapeutic agents and the one or more compatibilizers together in an organized composition. The chemotherapeutic agents and the one or more compatibilizers together can have a long-range order in the form of a repeating pattern. The chemotherapeutic agents and the one or more compatibilizers together can include a repetitive multi-drug motif (“MDM”) structure.

In some embodiments, the chemotherapeutic agent compositions in powder form do not include a structural feature of a lipid layer, a lipid bilayer, a liposome, a micelle, or any combination thereof. In some embodiments, the chemotherapeutic agent compositions are not amorphous (e.g., having a broad undefined X-ray diffraction pattern), but have discrete powder X-ray diffraction peaks indicative of organization and/or long-range order in the form of repeating patterns. In some embodiments, the chemotherapeutic agent compositions are not in the form of an implant (e.g., a subdermal implant). In some embodiments, the chemotherapeutic agent in the chemotherapeutic agent composition is present in its native, salt, or solvate form, but a prodrug thereof is not required to provide the long-acting injectable aqueous dispersion. In some embodiments, the chemotherapeutic agent compositions do not include nano/microcrystalline forms of the therapeutic agents or the compatibilizer(s).

In some embodiments, the chemotherapeutic agent composition of the present disclosure is not an amorphous solid dispersion. Rather, a given chemotherapeutic agent composition is not a physical mixture or a blend of its constituent chemotherapeutic agents and excipients, and as such, possesses properties unique to the composition that are different from those of each of the constituent chemotherapeutic agents and excipients. For example, the chemotherapeutic agent compositions can have a phase transition temperature different from the transition temperature of each individual component when assessed by differential scanning calorimetry. In some embodiments, one or more of the transition temperatures of each individual component is no longer present in the chemotherapeutic agent compositions, which include an organized assembly of the chemotherapeutic agent and excipient components (i.e., one or more compatibilizers). In some embodiments, the chemotherapeutic agent compositions have a homogeneous distribution of each individual therapeutic agent when viewed by scanning electron microscopy, such that each individual component is not visually discernible at 10-20 kV.

The chemotherapeutic agent compositions can remain stable when stored at 25° C. for at least 2 weeks (e.g., at least 3 weeks, at least 4 weeks, at least 6 weeks, or at least 8 weeks) and/or up to 12 months (e.g., up to 6 months, up to 6 months, or up to 4 months), at a relative humidity of 20% to 80%, at a pressure of 1 atm, and in air (i.e., 21% oxygen and 78% nitrogen), such that the at least one X-ray diffraction peak at position(s) corresponding to a given chemotherapeutic agent composition are preserved over the time period. In some embodiments, both the X-ray diffraction peak positions and intensities are preserved when the composition is stored at 25° C. for at least 2 weeks (e.g., at least 3 weeks, at least 4 weeks, at least 6 weeks, or at least 8 weeks) and/or up to 12 months (e.g., up to 6 months, up to 6 months, or up to 4 months).

In some embodiments, a given chemotherapeutic agent composition includes each chemotherapeutic agent in an amount of 2 wt % or more (e.g., 3 wt % or more, 5 wt % or more, 10 wt % or more, or 15 wt % or more) and 20 wt % or less (e.g., 15 wt % or less, 10 wt % or less, 5 wt % or less, or 3 wt % or less) relative to the weight of the total chemotherapeutic agent composition.

In some embodiments, the chemotherapeutic agent compositions can include a molar ratio of the sum of chemotherapeutic agents to the one or more compatibilizers of from about 1:10 (e.g., from about 1:9, from about 1:8, from about 1:7, from about 1:6, from about 1:5, from about 1:4, from about 1:3, or from about 1:2) to about 1:1 (e.g., to about 1:2, to about 1:3, to about 1:4, to about 1:5, to about 1:6, to about 1.7, to about 1:8, or to about 1:9). In certain embodiments, the chemotherapeutic agent compositions can include a molar ratio of the sum of chemotherapeutic agents to the one or more compatibilizers of from about 1:7 to about 1:2.

The chemotherapeutic agent compositions can be a solid. For example, the chemotherapeutic agent compositions can be a powder. The powder can be formed of particles having an average dimension of from 100 nm (e.g., from 500 nm, from 1 μm, from 4 μm, from 6 μm, or from 8 μm) to 10 μm (e.g., to 8 μm, to 6 μm, to 4 μm, to 1 μm, or to 500 nm). The average dimension (e.g., a diameter) of a particle can be determined by transmission and/or scanning electron microscopy, averaged over 500 particles. In some embodiments, particle diameter can be measured using photon correlation spectroscopy.

Aqueous Dispersions

The present disclosure also features injectable aqueous dispersions including an aqueous solvent, and a chemotherapeutic agent composition dispersed in the aqueous solvent to provide the injectable aqueous dispersion. The injectable aqueous dispersions exhibit a therapeutically effective plasma concentration of the combination of chemotherapeutic agents for 2 or more weeks (from a single injected bolus dose).

The chemotherapeutic agent composition can be in powder form prior to dispersion in the aqueous solvent to provide the aqueous dispersion. The powder form of the chemotherapeutic agent composition is described above. The chemotherapeutic agent composition powder can be mixed with an aqueous solvent to provide an aqueous dispersion. The aqueous dispersion can be a suspension of the chemotherapeutic agent composition. In some embodiments, once suspended in the aqueous solvent, the size of the suspended particles of the chemotherapeutic agent composition is reduced (e.g., to less than 0.2 μm) prior to administration to a subject, for example, by subjecting the aqueous dispersion to a homogenizer and/or a sonicator. The aqueous dispersion can then be optionally filtered to remove any microorganisms, for example, through a 0.2 μm filter. The aqueous dispersion is adapted to be parenterally administered to a subject. As used herein, parenteral administration refers to a medicine taken into the body or administered in a manner other than through the digestive tract, such as by intravenous or subcutaneous administration.

The chemotherapeutic agents in the chemotherapeutic agent compositions can be present at various molar ratios. For example, the combination of chemotherapeutic agents can include gemcitabine and paclitaxel, at a gemcitabine:paclitaxel molar ratio of from about 1:1 (e.g., from about 2:1, from about 5:1, from about 10:1, from about 20:1, from about 25:1, from about 30:1, from about 40:1, or from 45:1) to about 50:1 (e.g., to about 45:1, to about 40:1, to about 30:1, to about 25:1, to about 20:1, to about 10:1, to about 5:1, or to about 2:1). As another example, the combination of chemotherapeutic agents can include venetoclax and zanubrutinib, at a venetoclax: zanubrutinib molar ratio of from about 10:1 (e.g., from about 8:1, from about 6:1, from about 4:1, from about 2:1, from about 1:1, from about 1:2, from about 1:3, from about 1:5, from about 1:7, or from about 1:9) to about 1:10 (e.g., to about 1:9, to about 1:7, to about 1:5, to about 1:3, to about 1:2, to about 1:1, to about 2:1, to about 4:1, to about 6:1, to about 8:1).

The combination of chemotherapeutic agents at these ratios can exhibit sustained plasma concentrations of 2 weeks or more, 3 weeks or more, 4 weeks or more, 5 weeks or more, or 6 weeks or more, from a single injected bolus dose. As used herein, a sustained plasma concentration is a plasma drug concentration that is maintained for a defined period (e.g., 14 days or more and/or 90 days or less) above the EC₅₀ value of each chemotherapeutic agent in the combination of therapeutic agents, and at a dosage without adverse effects (e.g., pain and other untoward effects as defined in a clinical product label). The plasma drug concentration is determined from the blood taken from the subject over time and the drug levels determined with a validated assay in the plasma (separated from the coagulated blood and free of red cells).

In some embodiments, the injectable aqueous dispersions exhibit a therapeutically effective plasma concentration of the combination of chemotherapeutic agents for 2 or more weeks, from a single injected dose. In some embodiments, the injectable aqueous dispersions exhibit a therapeutically effective plasma concentration of the combination of chemotherapeutic agents for 3 or more weeks, from a single injected dose. In some embodiments, the injectable aqueous dispersions exhibit a therapeutically effective plasma concentration of the combination of chemotherapeutic agents for 4 or more weeks, after a single injected dose. In some embodiments, the injectable aqueous dispersions exhibit a therapeutically effective plasma concentration of the combination of chemotherapeutic agents for 5 or more weeks, after a single injected dose. In some embodiments, the injectable aqueous dispersions exhibit a therapeutically effective plasma concentration of the combination of chemotherapeutic agents for 6 or more weeks, after a single injected dose.

In the aqueous dispersion, the chemotherapeutic agents and the one or more compatibilizers together can form an organized composition, as discussed above. In the aqueous dispersion, the chemotherapeutic agents and the one or more compatibilizers together can have a long-range order in the form of a repeating pattern. In the aqueous dispersion, the chemotherapeutic agents and the one or more compatibilizers together can include a repetitive multi-drug motif (“MDM”) structure.

In some embodiments, the aqueous dispersions do not include a structural feature of a lipid layer, a lipid bilayer, a liposome, a micelle, or any combination thereof. The aqueous dispersions do not include a chemotherapeutic agent composition that is amorphous. In some embodiments, the aqueous dispersions are not in the form of nor incorporated in an implant (e.g., a subdermal implant). In some embodiments, the chemotherapeutic agent in the aqueous dispersions is present in its native, salt, or solvate form, but a prodrug thereof is not needed to provide the long-acting injectable aqueous dispersion. In some embodiments, the aqueous dispersions of the present disclosure do not include nano/microcrystalline forms of the therapeutic agents and/or the compatibilizer(s).

In some embodiments, the aqueous solvent is a buffered aqueous solvent, saline, or any balanced isotonic physiologically compatible buffer suitable for administration to a subject, as known to a person of skill in the art. For example, the aqueous solvent can be an aqueous solution of 10-100 mM (e.g., 20 mM, 40 mM, 60 mM, or 80 mM) sodium bicarbonate and 0.45 wt % to 0.9 wt % NaCl.

A given aqueous dispersion can include each chemotherapeutic agent in an amount of 5 wt % or more (e.g., 15 wt % or more, 20 wt % or more, or 25 wt % or more) and 30 wt % or less (e.g., 25 wt %, 20 wt % or less, or 15 wt % or less), relative to the final aqueous dispersion. In some embodiments, the aqueous dispersion can include the total chemotherapeutic agent composition in an amount of 5 wt % or more (e.g., 15 wt % or more, 20 wt % or more, or 25 wt % or more) and 30 wt % or less (e.g., 25 wt %, 20 wt % or less, or 15 wt % or less), relative to the final aqueous dispersion.

The aqueous dispersions of the chemotherapeutic agent composition of the present disclosure can provide a therapeutically effective plasma concentration of the chemotherapeutic agents over a longer period of time compared an aqueous dispersion of a physical mixture of the chemotherapeutic agents and excipients, an amorphous mixture of the therapeutic agents and excipients, or compared to separately administered chemotherapeutic agents at a same dosage. In some embodiments, the aqueous dispersions of the chemotherapeutic agent composition of the present disclosure can provide a therapeutically effective plasma concentration of the chemotherapeutic agents over a longer period of time and at a lower dosage compared an aqueous dispersion of a physical mixture of the chemotherapeutic agents and excipients, an amorphous mixture of the therapeutic agents and excipients, or compared to separately administered chemotherapeutic agents at a same dosage. In some embodiments, the aqueous dispersions of the chemotherapeutic agent composition provide from 2 (e.g., from 5, from 10, or from 15) to 50 (e.g., to 40, to 30, or to 20) fold higher exposure (e.g., AUC₀₋₂₄ₕ calculated from plasma drug concentrations using the trapezoidal rule) of each chemotherapeutic agent in the chemotherapeutic agent composition in non-human primates, when administered parenterally (e.g., subcutaneously), when compared to non-human primates treated with an equivalent dose of the same free and soluble therapeutic agent individually in solution. In some embodiments, the aqueous dispersions of the chemotherapeutic agent composition provide from 20-fold (e.g., from 30 fold, or from 40 fold) to 50 fold (e.g., to 40 fold, or to 30 fold) higher exposure (e.g., AUC₀₋₂₄ₕ calculated from plasma drug concentrations using the trapezoidal rule) of each chemotherapeutic agent in the chemotherapeutic agent composition in non-human primates, when administered parenterally (e.g., subcutaneously), when compared to non-human primates treated with an equivalent dose of the same free and soluble chemotherapeutic agent individually in solution.

In some embodiments, the aqueous dispersions of the chemotherapeutic agent compositions of the present disclosure are long-acting, such that the parenteral administration of the aqueous dispersion can occur once every 2 weeks (e.g., every 3 weeks, every 4 weeks, or every 5 weeks) to once every 6 weeks (e.g., every 5 weeks, every 4 weeks, or every 3 weeks).

In certain embodiments, the aqueous dispersions of the chemotherapeutic agent compositions of the present disclosure have a terminal half-life greater than the terminal half-life of each freely solubilized individual chemotherapeutic agent. For example, the chemotherapeutic agent compositions and aqueous dispersions thereof can have a half-life extension of greater than 2 to 3-fold of each constituent chemotherapeutic agent's individual elimination half-life. In some embodiments, the chemotherapeutic agent compositions and aqueous dispersions thereof can have a half-life extension of from 8-fold (e.g., from 10-fold, from 15-fold, from 20-fold, from 30-fold, from 40-fold, or from 50-fold) to 62-fold (e.g., to 50-fold, to 40-fold, to 30-fold, to 20-fold, to 15-fold, or to 10-fold) for each constituent therapeutic agent's individual elimination half-life.

The particles of chemotherapeutic agent compositions in the aqueous dispersion can maintain the MDM organization of the chemotherapeutic agents and the one or more compatibilizers, such that the physically-assembled stable molecular organization of the therapeutic agents and the compatibilizers is preserved. In some embodiments, the particles of the chemotherapeutic agent composition in the aqueous dispersion do not form a lipid layer, a lipid bilayer, a liposome, or a micelle in the aqueous solvent. In some embodiments, the particles of the chemotherapeutic agent composition in the aqueous dispersion do not include a nanocrystalline chemotherapeutic agent. In some embodiments, after hydration of the chemotherapeutic agent composition, the particles of chemotherapeutic agent compositions are discoidal rather than spherical, when visualized by transmission electron microscopy. For example, the discoid particles of the chemotherapeutic agent compositions, after suspension in an aqueous solvent, can have a dimension of, for example, a width of from 5 nm (e.g., from 8 nm, from 10 nm, or from 15 nm) to 20 nm (e.g., to 15 nm, to 10 nm, or to 8 nm) by a length of from 30 nm (e.g., from 35 nm, from 40 nm, or from 45 nm) to 50 nm (e.g., to 45 nm, to 40 nm, or to 35 nm), having a thickness of from 3 nm (e.g., from 5 nm, from 7 nm) to 10 nm (e.g., to 7 nm, to 5 nm), as visualized by transmission electron microscopy.

The particles of the chemotherapeutic agent composition in the aqueous dispersion can have a maximum dimension of from 10 nm (e.g., 25 nm, 50 nm, 100 nm, 150 nm, 200 nm) to 300 nm (e.g., 200 nm, 150 nm, 100 nm, 50 nm, or 25 nm). Particle diameter can be measured using photon correlation spectroscopy.

As used herein, the “aqueous dispersion” refers to a suspension of the chemotherapeutic agent composition in the aqueous solvent, where the chemotherapeutic agent composition is present in the form of insoluble particles suspended, stably in the aqueous solvent. In some embodiments, rather than an aqueous dispersion, the chemotherapeutic agent composition can be dissolved in an aqueous solvent to provide a solution. When the chemotherapeutic agent composition is in a solution, it is solubilized and dissolved in the solvent.

### Methods of Treatment

The present disclosure further provides a method of treating a cancer, in particular a cancer that expresses an upregulation of Bruton tyrosine kinase (BTK), Bcl-2, or both BTK and Bcl-2, by parenterally administering an injectable aqueous dispersion of a chemotherapeutic agent composition of the present disclosure. The chemotherapeutic agents of the chemotherapeutic agent composition exhibit a synergistic chemotherapeutic effect. In some embodiments, the chemotherapeutic agents of the chemotherapeutic agent composition exhibit a synergistic inhibitory effect on BTK, Bcl-2, or both BTK and Bcl-2. In some embodiments, the cancer includes metastatic breast cancer, lung cancer, pancreatic cancer, and/or a liquid tumor (e.g., leukemia). In some embodiments, the methods of the present disclosure inhibit metastasis of a cancer, such as breast cancer. In some embodiments, the methods of the present disclosure inhibit formation of lung metastasis nodules.

The dose of the injectable aqueous dispersion of the chemotherapeutic agent composition can be a bolus dose. As used herein, “parenteral administration” refers to a medicine taken into the body or administered in a manner other than through the digestive tract, such as by intravenous or subcutaneous administration. In some embodiments, parenteral administration does not include intramuscular administration.

For example, the methods can include parenterally administering to a subject in need thereof, at a frequency of at most one dose every 2 weeks (e.g., at most one dose every 3 weeks, at most one dose every 4 weeks, at most one dose every 5 weeks, or at most one dose every 6 weeks) an aqueous dispersion including an aqueous solvent, and a chemotherapeutic agent composition dispersed in the aqueous solvent.

As discussed above, in some embodiments, the chemotherapeutic agent composition includes a combination of chemotherapeutic agents, such as a combination of gemcitabine and paclitaxel, or a combination of venetoclax and zanubrutinib. The chemotherapeutic agent compositions further include one or more compatibilizers including a lipid(e.g., a lipid excipient), a lipid conjugate, or a combination thereof.

In some embodiments, the method of treating cancer includes administering a chemotherapeutic composition at a gemcitabine dosage of from 1 mg/kg (e.g., 10 mg/kg, 20 mg/kg, 30 mg/kg, or 40 mg/kg) to 50 mg/kg (e.g., 40 mg/kg, 30 mg/kg, 20 mg/kg, or 10 mg/kg) and a paclitaxel dosage of from 0.1 mg/kg (e.g., 1 mg/kg, 5 mg/kg, 10 mg/kg, 20 mg/kg, 30 mg/kg, or 40 mg/kg) to 50 mg/kg (e.g., 40 mg/kg, 30 mg/kg, 20 mg/kg, 10 mg/kg, 5 mg/kg, or 1 mg/kg). In some embodiments, when the chemotherapeutic agent composition includes gemcitabine and paclitaxel, the composition exhibits an AUC of from 1,000 μg·min/mL (e.g., 5,000 μg·min/mL, 10,000 μg·min/mL, 20,000 μg·min/mL, 30,000 min/mL, 40,000 μg·min/mL, or 50,000 μg·min/mL) to 60,000 μg·min/mL (e.g., 50,000 μg·min/mL, 40,000 μg·min/mL, 30,000 μg·min/mL, 20,000 μg·min/mL, 10,000 min/mL, or 5,000 μg·min/mL) for gemcitabine and an AUC of from 150 μg·min/mL (e.g., 300 μg·min/mL, 600 μg·min/mL, or 800 μg·min/mL) to 1,000 μg·min/mL (e.g., 800 min/mL, 600 μg·min/mL, or 300 μg·min/mL) for paclitaxel.

In some embodiments, the method of treating cancer includes administering a chemotherapeutic composition at a venetoclax dosage of from 0.1 mg/kg (e.g., from 1 mg/kg, from 5 mg/kg, from 10 mg/kg, from 15 mg/kg, from 20 mg/kg, or from 25 mg/kg) to 30 mg/kg (e.g., to 25 mg/kg, to 20 mg/kg, to 15 mg/kg, to 10 mg/kg, to 5 mg/kg, or to 1 mg/kg) and a zanubrutinib dosage of from 0.1 mg/kg (e.g., from 1 mg/kg, from 5 mg/kg, from 10 mg/kg, from 15 mg/kg, from 20 mg/kg, or from 25 mg/kg) to 30 mg/kg (e.g., to 25 mg/kg, to 20 mg/kg, to 15 mg/kg, to 10 mg/kg, to 5 mg/kg, or to 1 mg/kg). In some embodiments, when the chemotherapeutic agent composition includes venetoclax and zanubrutinib, the composition exhibits an AUC of from 150 μg.h/mL (e.g., 200 μg.h/mL, 300 μg.h/mL, or 400 μg.h/mL) to 500 μg.h/mL (e.g., 400 μg.h/mL, 300 μg.h/mL, or 200 μg.h/mL) for venetoclax and an AUC of from 10 μg.h/mL (e.g., 25 μg.h/mL, 50 μg.h/mL, or 75 μg.h/mL) to 100 μg.h/mL (e.g., 75 μg.h/mL, 50 μg.h/mL, or 25 μg.h/mL) for zanubrutinib.

In certain embodiments, the paclitaxel in the aqueous dispersions of the chemotherapeutic agent compositions of the present disclosure exhibits an apparent terminal half-life of from 1.5 hours (h) (e.g., from 2 h, from 3 h, or from 4 h) to 5 h (e.g., to 4 h, to 3 h, or to 2 h). In certain embodiments, the gemcitabine in the aqueous dispersions of the chemotherapeutic agent compositions of the present disclosure exhibits an apparent terminal half-life of from 5 h (e.g., from 8 h, from 10 h, or from 15 h) to 20 h (e.g., to 15 h, to 10 h, or to 8 h). In certain embodiments, the venetoclax in the aqueous dispersions of the chemotherapeutic agent compositions of the present disclosure exhibits an apparent terminal half-life of from 24 h (e.g., from 36 h, from 48 h, or from 60 h) to 75 h (e.g., to 60 h, to 48 h, to 36 h). In certain embodiments, the zanubrutinib in the aqueous dispersions of the chemotherapeutic agent compositions of the present disclosure exhibits an apparent terminal half-life of from 24 h (e.g., from 36 h, from 48 h, or from 60 h) to 80 h (e.g., to 60 h, to 48 h, to 36 h).

In some embodiments, the aqueous dispersion exhibits a 1-fold or more (e.g., 5-fold or more, 10-fold or more, 30-fold or more, 45-fold or more) to 60-fold or less (e.g., to 45-fold or less, 30-fold or less, 10-fold or less, or 5-fold or less) the AUC of each chemotherapeutic agent in mice, when administered subcutaneously, compared to the exposure of each freely solubilized or suspended individual chemotherapeutic agent. In some embodiments, each chemotherapeutic agent in the combination of chemotherapeutic agents of the aqueous dispersion has a terminal half-life greater than the terminal half-life of each freely solubilized or suspended individual therapeutic agent.

In some embodiments, the aqueous dispersion exhibits a therapeutic index of greater than 1.5 (e.g., greater than 2, greater than 3, greater than 4, greater than 5, greater than 6, greater than 7, greater than 8, greater than 9, or greater than 10). In some embodiments, the aqueous dispersion exhibits a therapeutic index of 5-10.

In some embodiments, parenteral administration of the aqueous dispersion to the subject occurs at a frequency of at most one dose per every week. In some embodiments, parenteral administration of the aqueous dispersion to the subject occurs at a frequency of at most one dose per every 2 weeks. In some embodiments, parenteral administration of the aqueous dispersion to the subject occurs at a frequency of at most one dose per every 3 weeks. In some embodiments, parenteral administration of the aqueous dispersion to the subject occurs at a frequency of at most one dose per every 4 weeks. In some embodiments, parenteral administration of the aqueous dispersion to the subject occurs at a frequency of at most one dose per every 5 weeks. In some embodiments, parenteral administration of the aqueous dispersion to the subject occurs at a frequency of at most one dose per every 6 weeks.

In some embodiments, the aqueous dispersion is administered intravenously. In some embodiments, the aqueous dispersion is administered subcutaneously. In some embodiments, the aqueous dispersion is not administered intramuscularly.

### Methods of Making the Aqueous Dispersions

General Procedure

The process of making an injectable aqueous dispersion including a chemotherapeutic agent composition that includes water-soluble and water-insoluble chemotherapeutic agents (to provide long-acting pharmacokinetic characteristics) can generally be performed in three steps.

Step 1—Production of the Chemotherapeutic Agent Composition in Powder Form

1, 2, or 3 therapeutic agents from the water insoluble category, such as paclitaxel, venetoclax, and/or zanubrutinib in solid states, can first be dissolved together with one or more compatibilizers (e.g., DSPC and mPEG₂₀₀₀-DPSE) in a container with alcoholic solvent at a temperature 60-90° C. Then water-soluble drugs such as gemcitabine (e.g., at a concentration of about 10 to 50 mg/ml) were prepared in buffered aqueous solution at pH 5-8 (e.g., a 0.45 (w/v)% NaCl buffered aqueous solution) at 60-90° C. Then the water-soluble drugs in buffered solution are added drop-wise into water insoluble drugs which are fully dissolved in ethanol at 60-90° C. such that the final total solid concentration in the ethanol-water (9:1 v/v) solution is 5-10 (w/v)%. In some embodiments, the therapeutic agents and the compatibilizers can be dissolved together in an alcohol at elevated temperatures (e.g., ethanol at 60-90° C.). When all component-drugs and lipids are in solution, the mixture can be spray-dried (e.g., with Procept M8TriX (Zelzate, Belgium) or Buchi B290) or otherwise lyophilized. For example, for Procept instruments, inlet temperature for the spray dryer can be maintained at 70° C. with an inlet air speed of 0.3 m³/min and chamber pressure of 25 mBar. Dried drug combination nanoparticle powder generated by the spray-dryer can be collected; and subjected to vacuum desiccation. The dried powder chemotherapeutic agent composition can be characterized with powder X-ray diffraction to be free of individual drug crystal signatures, but with a cohesive unified X-ray diffraction pattern representing multiple drug (combination) domains (MDM) assembled in repeating units. The MDM diffraction pattern can be different from that of amorphous X-ray diffraction presented typically as a broad halo with no single peak in the drug powder products. In addition, in contrast to a metastable state of amorphous organization that return to individual drug x-ray signatures of crystalline form, the single unified peak in the X-ray diffraction for the chemotherapeutic agent composition powder, which was contributed by MDM ordering, can be stable at 25-30° C. for months (e.g., more than 6 months, more than 9 months, more than 12 months).

Step 2—Production of the Aqueous Dispersion

The powder chemotherapeutic agent composition can be resuspended in buffer (e.g., 0.45 NaCl containing 50 mM NaHCO₃, pH 7.5) at 65-70° C. to provide an aqueous suspension. After the powder is in suspension, the mixture can be allowed to hydrate (absorbing water to DcNP powder containing MDM structure) with mixing at elevated temperatures (e.g., 65-70° C. for 2-4 hours, pH 7-8). The suspension can be subjected to size reduction (e.g., with a homogenizer until a uniform particle size between 10 nm and 300 nm mean diameter). Particle diameter can be measured using photon correlation spectroscopy.

Step 3—Sterile Injectable Aqueous Dispersion

To produce a sterile injectable suspension, the suspension can be sterilized using methods known to a skilled practitioner. For example, the step 2 process can be performed either under aseptic conditions in a class II biosafety sterile cabinet or the aqueous dispersion can be filtered through 0.2 μm terminal sterilization filter. The final injectable aqueous dispersion can be collected in a sterile glass vial; sterility can be verified by exposing the product on a blood agar plate test for 7 days with no bacterial growth.

### Bioanalytical Assays to Determine Therapeutic Agent Concentration

Plasma therapeutic agent concentrations can be measured using an assay developed and validated previously (see, e.g., Kraft et al., J Control Release. 2018 Apr. 10; 275: 229-241, incorporated herein by reference in its entirety). The lower limit of quantification can be 0.01 nM for the therapeutic agents in plasma.

Effects of the Injectable Aqueous Dispersion on Chemotherapeutic Drug Combinations in Mice

Mice can be intravenously administered with a control or an aqueous dispersion of the present disclosure. Blood can be collected through retro-orbital bleeding at predetermined time intervals. Each group can have a number of animals and each animal can be bled once only. Retro-orbital blood collection can be a terminal procedure. After blood collection, mice can be euthanized by CO₂ overdose followed by cervical dislocation as the secondary method of euthanasia. Drugs in plasma can be extracted and analyzed by LC-MS/MS as described below.

### Drug Extraction

A liquid-liquid extraction can be used to extract drugs from plasma or tissue homogenates. The samples can be diluted with a blank matrix to an appropriate concentration range. Samples can be spiked by internal standards followed by the addition of acetonitrile. Samples can then vortexed and centrifuged at 4° C. for an appropriate amount of time at a predetermined rpm. The supernatant can be removed and dried under nitrogen. The dried samples can be reconstituted in 20% methanol and 80% water.

### Quantification of Drugs by LC-MS/MS

Drugs can be quantified using HPLS coupled to a mass spectrometer. Chromatographic separation of drugs can be carried out as well known to a person of skill in the art. Analytes can be monitored using multiple-reaction monitoring for positive ions.

### 4T1 Cell Inoculation

4T1 cells can be transfected with luciferase and green fluorescence protein (GFP) (4T1-luc); thus, 4T1 growth could be monitored based on that bioluminescence. 4T1-luc suspended in buffer can be intravenously inoculated through mouse tail veins. Mice can be monitored for a predetermined period. Bioluminescence of 4T1-luc from living mice can be examined by an imaging system as known to a person of skill in the art. Mice can receive D-luciferin through intraperitoneal injections 10-15 min before imaging.

### Effects of Chemotherapeutic Agents on Metastatic Breast Cancer Colony Formation in the Lung

Mice can be inoculated with 4T1-luc cells IV in buffer on day 0. Three hours later, mice can be giving a single administration of saline, a control, or an aqueous dispersion of chemotherapeutic agent combinations of the present disclosure. On day 14, mice can be euthanized immediately after live imaging and lungs can be collected and placed in 12-well plates to quantify luminescence images. Mouse lung tissue can be fixed in formalin and stored in 70% EtOH before being embedded in paraffin blocks. GFP staining of thin sections can be carried out.

### Statistical Analysis

Students' t-tests can be performed, and the statistical significance can be evaluated using one-way ANOVA for multiple groups. A P-value of <0.05 can be considered statistically significant. Statistical analyses can be performed using GraphPad Prism.

### Assessing Drug Potency Against Liquid Cancer Cell Growth

K-562 cells (human leukemia), MOLT-4, and HL-60 cell lines can be used. The cells can be cultured in Gibco RPMI medium 1640 with Gibco 1% 100×Antibiotic-Antimycotic (Thermo Fisher Scientific, Waltham, USA) and 10% fetal bovine serum. Cells can be selected for their different protein expression levels of Bruton's Tyrosine Kinase (BTK) and B Cell Lymphoma 2 (Bcl-2); HL-60 cells express both BTK and Bcl-2, while K-562 and MOLT-4 cells only express BTK and Bcl-2, respectively.

Each cell line can be seeded separately into Costar® Black 96-well Assay Plates (Corning USA). Within 1 hr, varying concentrations of individual free drug, a combination of free drugs (w/w 1:1), or a combination of drugs according to the aqueous dispersions of the present disclosure can be added to the cells. Following a 5-day incubation, growth of treated cells can be compared to untreated cells, quantified using an AlamarBlue Cell Viability Assay (Thermo Fisher Scientific, Waltham, USA) with a plate reader. Prism graphing software (GraphPad) can used to analyze the absorbance data and to assess relative cell growth.

### Leukemic Cell Uptake and Retention of Therapeutic Agents.

HL-60 cells can be cultured, counted, and aliquoted into multiple Eppendorf tubes. A free drug solution of therapeutic agents (1:1 w/w) was added to half of the tubes, while an aqueous dispersion of the present disclosure of identical drug concentrations can be added to the second half of tubes. The cells in the tubes can be allowed to incubate normally. At preselected time points, one incubation tube from each group can be removed from the incubator, and the cells inside were washed twice with media to remove external drug. Cells can be lysed with acetonitrile, and drug concentrations can be quantified according to the aforementioned extraction and LC-MS/MS protocol.

The Examples below describe chemotherapeutic agent compositions and injectable aqueous dispersions of the present disclosure.

### EXAMPLES

### Example 1 Drug Combination Nanoparticles Exhibiting Enhanced Plasma Exposure and Dose-Responsive Effects on Eliminating Breast Cancer Lung Metastasis

A novel drug combination of gemcitabine and paclitaxel (GT) was developed and evaluated. Leveraging a simple and scalable drug-combination nanoparticle platform (DcNP), an injectable GT combination in DcNP (GT DcNP) was successfully prepared. Compared to a Cremophor EL/ethanol assisted drug suspension in buffer (CrEL), GT DcNP exhibits about 56-fold and 8.6-fold increases in plasma drug exposure (area under the curve, AUC) and apparent half-life of gemcitabine respectively, and a 2.9-fold increase of AUC for paclitaxel. Using 4T1 as a syngeneic model for breast cancer metastasis, a single GT (20/2 mg/kg) dose in DcNP nearly eliminated colonization in the lungs. This effect was not achievable by a CrEL drug combination at a 5-fold higher dose (i.e., 100/10 mg/kg GT). A dose-response study indicates that GT DcNP provided a therapeutic index of ˜15.8. Collectively, these data suggest that GT DcNP could be effective against advancing metastatic breast cancer with a margin of safety. As the DcNP formulation is intentionally designed to be simple, scalable, and long-acting, it can be suitable for clinical development to find effective treatment against metastatic breast cancer.

Whether gemcitabine (G, soluble) and paclitaxel (T, insoluble) can be assembled into a drug-combination particle able to enhance pharmacokinetics and also inhibit the growth of metastatic breast cancer was investigated. The results demonstrate that a single dose of DcNP formulated GT combination (20/2 mg/kg GT in DcNP) could reduce 4T1 to nearly non-detectable levels by day 14, while there was little to no effect on 4T1 with equivalent CrEL drug dosing.

### Reagents and Cell Line

1,2-Distearoyl-sn-glycero-3-phosphocholine (DSPC) and N-(carbonylmethoxypolyethyleneglycol with MW=2000)-1,2-distearoyl-sn-glycero-3-phosphoethanolamine, sodium salt (DSPE-mPEG₂₀₀₀) (GMP grade) were purchased from Corden Pharma (Liestal, Switzerland). Paclitaxel (>99.5%), gemcitabine free form (>99%), and gemcitabine hydrochloride (>99%) were purchased from LC Laboratories (Woburn, Mass.). All other chemicals and reagents were analytical grade or higher. 4T1 cell line transfected and verified to express luciferase and green fluorescence protein (GFP) (referred to as 4T1-luc) was provided by Stanley Riddell laboratory, Fred Hutchinson Cancer Research Center.

### Formulation and Characterization of GT DcNP

DcNP composed of DSPC and DSPE-mPEG₂₀₀₀ as lipid excipients, paclitaxel, and gemcitabine (90:10:2.5:80 molar ratio) were prepared aseptically as follows: Lipid excipients and drugs were solubilized together in ethanol at 60° C. Ethanol was removed by controlled solvent evaporation at 60° C., followed by vacuum desiccation to remove residual solvent. The dry film was rehydrated to 100 mM lipids in 0.45% NaCl with 20 mM NaHCO₃ buffer at 60° C. for 2 h. Particle size was reduced at ˜40° C. using a bath sonicator (Avanti Polar Lipids, Inc. Alabaster, Ala.) (5 min on, 5 mM off, 3 cycles). GT DcNP formulations were stored at room temperature for further use. Particle size was determined by a NICOMP 380 ZLS (Particle Sizing Systems, Santa Barbara, Calif.). Drug extraction with acetonitrile followed by HPLC were used to quantify drugs in formulations. Drug association was measured by dialysis (6-8 kDa) of DcNP against 0.9% NaCl 20 mM NaHCO₃ buffer for 4 h and quantification by HPLC.

### Preparation of GT CrEL Drug Combination

Paclitaxel was dissolved in ethanol (20 mg/mL) and diluted with an equal volume of Cremophor EL (Sigma-Aldrich, St. Louis, Mo.). The solution was then diluted 10× with a premade PBS solution of gemcitabine (hydrochloride salt) (12.65 mg/mL). Final concentrations of drugs were 10/1 mg/mL GT. CrEL drug suspensions were used within the same day of preparation due to instability.

### Pharmacokinetic Study

All animal studies were conducted in accordance with University of Washington Institute of Animal Care and Use Committee (IACUC) approved protocols (protocol number 2372-06). Isoflurane was used for anesthesia during live animal imaging. 5-6 week-old female BALB/c mice were purchased from The Jackson Laboratory (Bar Harbor, Me.) and housed in an animal research facility for at least one week before use.

Mice were administered with either a CrEL drug combination or GT DcNP intravenously with doses of 50/5 mg/kg GT. Blood was collected through retro-orbital bleeding at 5, 60, 120, 360 mM for CrEL GT and 5, 60, 120, 360, 1440, 4320 min (72 h) for GT DcNP. Each group had three animals and each animal was bled once only. Retro-orbital blood collection in this study was a terminal procedure and animals were under anesthesia at the time of bleeding. After blood collection, mice were euthanized by CO₂ overdose followed by cervical dislocation as the secondary method of euthanasia. Drugs in plasma were extracted and analyzed by LC-MS/MS as described below.

### Drug Extraction

A liquid-liquid extraction was used to extract drugs from plasma or tissue homogenates. 50 μL of sample were transferred into 1.5 mL tubes with or without dilution by blank matrix to an appropriate concentration range. Samples were spiked by internal standards (see below) followed by the addition of acetonitrile. Samples were then vortexed and centrifuged at 4° C. for 15 minutes at 14000 rpm. The supernatant was then removed and dried under nitrogen at 40° C. The dried samples were reconstituted in 20% methanol and 80% water in 50 μL.

### Quantification of Drugs by LC-MS/MS

Drugs were quantified by a Shimadzu HPLC system coupled to a 3200 QTRAP mass spectrometer (Applied Biosystems, Grand Island, N.Y.). The HPLC system consisted of two Shimadzu LC-20A pumps, a DGU-20A5 degasser, and a Shimadzu SIL-20AC HT autosampler. The mass spectrometer was equipped with an electrospray ionization (ESI) TurbolonSpray source. The system was operated with Analyst software, version 1.5.2 (ABSciex, Framingham, Mass.).

Chromatographic separation of drugs was achieved using a Synergi column (100×2.0 mm; 4-μm particle size) with an inline C8 guard column (4.0×2.0 mm) (Phenomenex, Torrance, Calif.). An ammonium acetate buffer/reagent alcohol gradient was used to separate components. Analytes were monitored using multiple-reaction monitoring for positive ions. The following ion transitions were monitored: gemcitabine, m/z 264.066→112.000; paclitaxel, m/z 854.266→286.200; a stable labeled isotope (C₈¹³CH₁₂ClF₂N¹⁵N₂O₄) (m/z 267.067→415.100) was used as an internal standard for gemcitabine; docetaxel (m/z 830.312→549.3) was used as an internal standard for paclitaxel.

### 4T1 Cell Inoculation

Six-week-old, female BALB/c mice were used in this study. 4T1 cells were transfected with luciferase and green fluorescence protein (GFP) (4T1-luc); thus, 4T1 growth could be monitored based on that bioluminescence. 4T1-luc (0.5, 1 or 2×10⁵ cells) suspended in a 100 μL ice-cold HBSS suspension was intravenously inoculated through mouse tail veins. Mice were monitored for a two-week period. Bioluminescence of 4T1-luc from living mice was examined by a XENOGEN IVIS 200 imaging system (PerkinElmer, Inc. Waltham, Mass.). Mice received 150 mg/kg D-luciferin through intraperitoneal injections 10-15 min before imaging. The bioluminescence imaging parameters for living mice were set as follows: field of view, 12; excitation filter, closed; emission filter, open; exposure time, 120 sec; binning factor, 4; f/stop, 2. Total 4T1-luc bioluminescence emission from living mice was integrated using Live Image software (PerkinElmer, Waltham, Mass.).

### Effects of CrEL Drug Combinations and DcNP on Metastatic Breast Cancer Colony Formation in the Lung

Six-week-old, female BALB/c mice were inoculated with 2×10⁵ 4 T1-luc cells IV in 100 μL HBSS on day 0. Three hours later, mice were given a single administration of saline, a CrEL drug combination, or GT DcNP through IV injections (n=8-15). The GT doses were 50/5 mg/kg for CrEL and DcNP formulations. On day 14, mice were euthanized immediately after live imaging and lungs were collected and placed in 12-well plates to quantify luminescence images. The images were acquired by a Xenogen IVIS-200. The bioluminescence imaging parameters for living mice were set as follows: field of view, 24; excitation filter, closed; emission filter, open; exposure time, 180 sec; binning factor, 4; f/stop, 2. The imaging parameters for lungs were set as follows: field of view, 10; excitation filter, closed; emission filter, open; exposure time, 30 sec; binning factor, 4; f/stop, 2. Bioluminescence intensity from living mice and lungs was integrated using Live Image software. Mouse lung tissue was fixed in formalin and stored in 70% EtOH before being embedded in paraffin blocks. GFP staining of thin sections (5 μm) was carried out by UW histology and imaging core.

### Dose Dependence of GT DcNP on 4T1 Metastases

Six-week-old, female BALB/c mice were inoculated with 2×10⁵ 4 T1-luc cells in 100 μL HBSS through IV injections on day 0. Three hours later, mice received a single administration of saline or a different dosage of GT DcNP through IV injections (n=8-15). The dosages for DcNP were 0.125/0.0125, 1.25/0.125, 5/0.5, 10/1, and 20/2 mg/kg GT, respectively. Mouse behavior and overall health conditions were observed on a daily basis and body weight was measured every 2 days. On day 14, bioluminescence from living mice was examined with the IVIS imaging system as described above. Lung metastasis was detected by isolating lungs and imaging with IVIS as described above. After euthanasia, all organs were collected and visually examined for apparent toxicity.

### Statistical Analysis

Data were presented as mean±standard error of the mean (SEM). The number of mice in all groups ranges from 8 to 15. Students' t-tests were performed for two groups, and the statistical significance was evaluated using one-way ANOVA for multiple groups. A P-value of <0.05 was considered statistically significant. Statistical analyses were performed using GraphPad Prism (Version 7.0).

### Development Characterization of Gemcitabine and Paclitaxel Together in an Injectable DcNP Formulation

Whether a water soluble gemcitabine (Log P=−1.5) and insoluble paclitaxel (Log P=3) could be integrated into a drug combination particle in suspension presented as an injectable dosage form was investigated. At a fixed G:T 32:1 (m/m; equals to 10:1 w/w) and lipid excipients-DSPC, DSPE-mPEG₂₀₀₀ (9:1 m/m), a stable and scalable DcNP with approximately 60 nm diameter can be made. At least 4 batches of DcNP preparation were tested and this process was reproducible, the DcNP products were stable, and could be scaled-up for the in vivo studies described. As the resulting GT DcNP product was less than 200 nm in diameter and stable in suspension (for at least 3 months and amenable for sterilization by 0.2 μm filtration), it was suitable for IV administration. Since the current clinical dose for GT was approximately 10:1 (w/w) (gemcitabine 1000˜1250 mg/m², paclitaxel 80˜175 mg/m²), the DcNP was used with a similar drug ratio for the studies in mice described below.

Enhanced Plasma Gemcitabine and Paclitaxel Exposure when Presented in DcNP Dosage Form

The effect of GT DcNP on a plasma drug-concentration time course of co-formulated GT as injectable dosage form was determined. Compared to a CrEL drug combination counterpart, the two drugs in the GT DcNP formulation greatly improve the total plasma drug exposure of GT at an equivalent dose. After a 50/5 (GT) mg/kg IV dose, gemcitabine in DcNP exhibited about 56-fold higher exposure (AUC) and 8.6-fold longer apparent half-life than an equivalent CrEL drug combination dosage in mice (Table 1). The dramatic increase in gemcitabine AUC per dose was reflected in both a small (˜10%) increase in Cₘₐₓ and an ˜8.7-fold increase in apparent half-life. At a 10-fold lower dose than gemcitabine, paclitaxel in the fixed dose combination (5 mg/kg) given in GT DcNP exhibited an ˜21% decrease in Cₘₐₓ but a similar half-life (1.97 vs 1.81 h). Due to the higher persistence of paclitaxel in DcNP, the overall AUC enhancement was about 2.9 fold (Table 1). Collectively, these data indicated that a co-formulation of GT in DcNP provided longer acting and higher GT exposure in mice compared to CrEL drug dosages.

TABLE 1
The effect of gemcitabine and paclitaxel presented in a drug combination
nanoparticle platform (DcNP) dosage form on the select pharmacokinetic
parameters of the two drugs, compared to a CrEL drug dosage control form*.

| | Gemcitabine (G) | | Paclitavel (T) | |
|---|---|---|---|---|
| | CrEL | DeNP^{a} | CrEL | DcNP |
| Cₘₐₓ (μg/mL) | 165.12^{b} | 181.4 | 17.7 | 13.9 |
| AUC_{0→t} (μg · min/mL) | 917.6 | 52063.7 | 149.29 | 588.75 |
| t_{1/2apparent} (h) | 1.60 | 13.72 | 1.81 | 1.97 |

*GT (50/5 mg/kg in 100 μL) in DcNP or CrEL drug dosage form was given intravenously to mice. Plasma drug concentrations were determined over 3 days. The plasma drug concentration time course was analyzed and the listed pharmacokinetic parameters are generated based on non-compartmental analysis (n = 3 composite sampling).
^{a}CrEL drugs scaled to equivalent DcNP dosages.
^{b}Geometric Mean (95% CI).
Abbreviations: Cₘₐₓ, maximum plasma drug concentration; AUC_{0→t}, area under the plasma drug concentration-time curve to experimental time point; t_{1/2}, apparent terminal plasma drug half-life.

### Characterization of 4T1-Luc in BALB/c Mice as a Syngeneic Metastatic Tumor Establishment and Nodule Growth Model for Intervention Studies.

To determine whether enhanced GT exposure could translate into improvements in inhibition of metastatic tumor establishment and growth, if 4T1 inoculated intravenously into BALB/c mice could model hematogenous metastasis was evaluated. 4T1 introduced into the blood have recently been shown to establish and grow in the lungs as nodules, and at a much faster rate than in the lymph nodes and the sinuses. In addition, the 4T1 (labeled with luciferase for live tracking) were able to invade blood capillaries in the nodes and spread to lungs, which were detectable as 4T1 nodules. Therefore, a titration study to find a dose of 4T1 cells that produces tumor nodules in the lungs while not overburdening the mice with tumors was first performed. To do so, a 4T1 cell line carrying luciferase marker (4T1-luc) was used. The transfection of luciferase did not affect cell proliferation or migration. After verifying luciferase expression by the 4T1-luc, these breast cancer cells were inoculated into the tail veins of BALB/c female mice. A dose range between 50 to 200×10³ 4 T1-luc cells in the inoculum per mouse) was studied. Bioluminescence (of 4T1-luc), body weight, and general behavior were monitored over two weeks. Bioluminescence signals increased exponentially from days 10 to 13 (from 0.5 to 3.5×10⁵ photo counts). Furthermore, the body weight of mice gradually declined (˜10% from day 10 to day 13) at a higher inoculum dose in mice corresponding to the exponential increase in the lung bioluminescence intensity. With 200×10³ cells, about 20˜30 tumor nodules and 2.0˜3.0×10⁵ photon counts of bioluminescence could be detected in the mouse lungs—showing that colonies establish and grow over time in these tissues with acceptable weight and overall health for interventional studies. Thus, 200×10³ 4 T1-luc cells in the inoculum was used for the following studies.

To verify the reproducibility of this model, the study was repeated five times with a total of 21 mice and lung bioluminescence intensities were compared using a 200×10³ cell inoculation number. Results indicate that the model was highly reproducible and reliable with 100% tumor uptake and no significant difference between the mean bioluminescence (p=0.0681 by one-way ANOVA). The rapid and aggressive 4T1 tumor growth at this dose limited the ability to keep untreated mice for up to 14 days. The effectiveness studies in following sections were determined using a 200×10³ 4 T1 cell inoculation while carrying saline controls for each set of experiments or replications.

### Effects of DcNP on Gemcitabine and Paclitaxel Combinations for Inhibiting 4T1 Syngeneic Mouse Metastasis

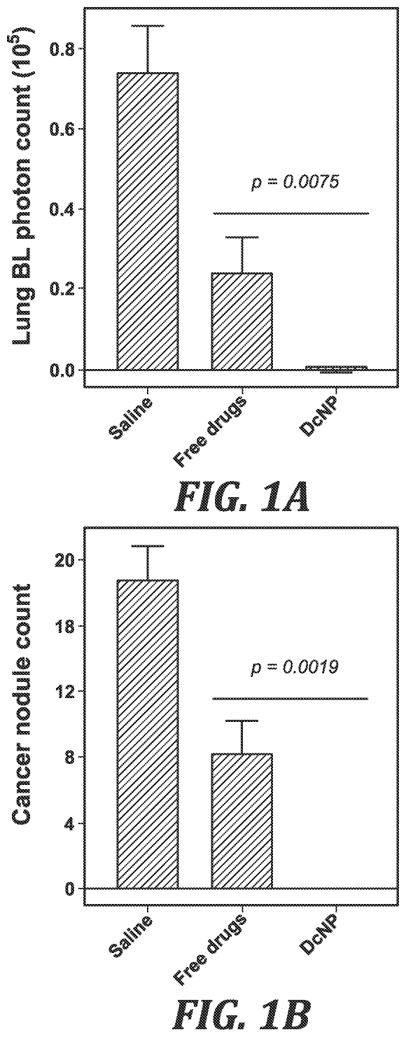

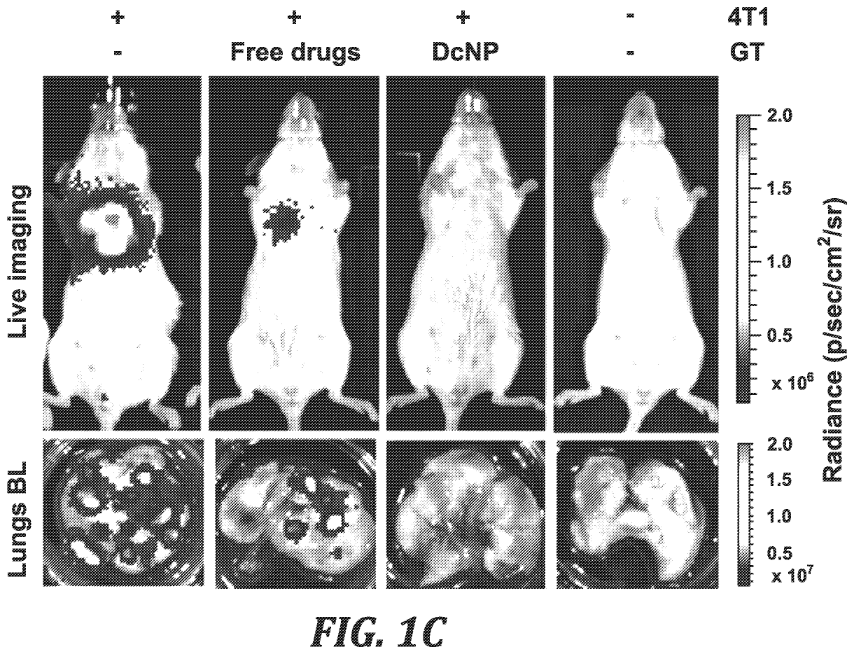

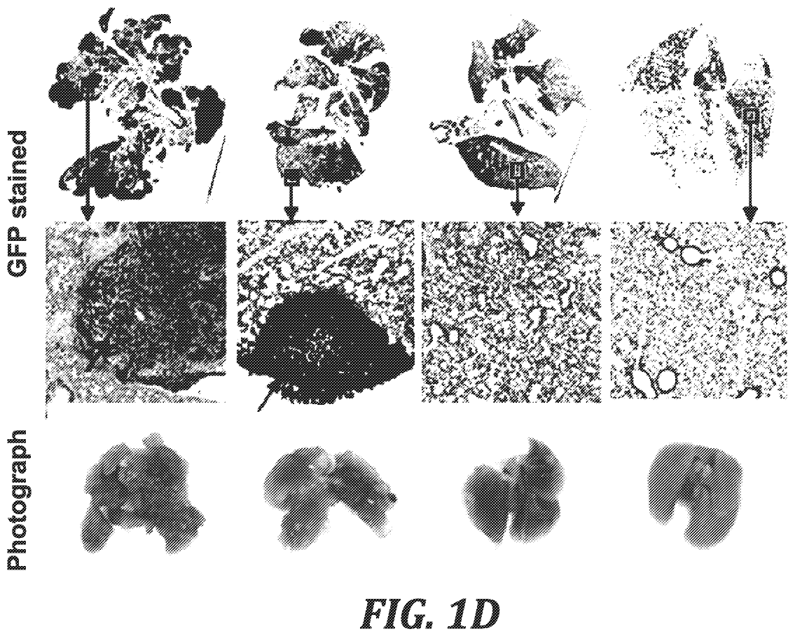

To determine the effects of enhanced GT exposure when presented in DcNP dosage form, 50/5 mg/kg (GT) was first based on the current clinical (surface area converted to weight based) dose. Mice were first inoculated with 4T1 cells and given a single IV dose of GT either in CrEL or DcNP form. Identical GT doses (50/5 mg/kg) were chosen for the two formulations to evaluate DcNP effect on this treatment model (and without using dose compensations to match plasma drug exposures between the two formulations). The short interval between cell inoculation and GT administration (3h) was also purposefully designed, as the goals of this study were examining the clearance of advancing cancer cells in blood and eliminating formation of lung metastasis nodules. Tumor nodule formation was monitored over 14 days. As shown in FIGS. 1A and 1B, at day 14 mice treated with GT DcNP formulation completely inhibited 4T1 colonies in the lungs while the CrEL dosage form only inhibited 60˜70%. The bioluminescence intensity data are verified with lung nodule counts and ex-vivo 4T1-luc-luminescence verification of the excised lungs (FIG. 1C). However, a trend toward weight loss around day 4-6 in mice treated with 50/5 GT mg/kg or higher dose was observed. These quantitative data indicate that a single dose of GT co-formulated in DcNP could completely inhibit the establishment and growth of 4T1 metastatic cancer in the lungs at a significantly higher rate than that provided by CrEL drug combinations in this syngeneic mouse model.

To further characterize lung metastasis and treatments at the microscopic level, lung sections were examined with GFP immunohistochemistry given GFP is expressed by 4T1-luc, and the microstructures were evaluated in comparison to controls (FIG. 1D). First, the metastatic nodules in lungs often occur near blood vessels, indicating that 4T1-luc cells deposit in lungs following deposition in narrow vasculature. Second, the lung tissues of 4T1 inoculated mice treated with placebo or drug combinations was evaluated. As shown in FIG. 1D (second rows of graphs), the cross-sections of lungs treated with CrEL dosage form and placebo exhibited a larger number of 4T1 cells positive to GFP while those from a GT DcNP single dose treatment did not. These data were consistent with bioluminescence measures in-life and ex-vivo of lung analysis where a high single IV dose GT DcNP treatment completely cleared 4T1 cancer nodules in the lungs.

### Dose-Dependent Tumor Inhibitory and Gross Toxicity (Weight Loss) Effect of GT Combination in DcNP to Estimate Therapeutic Index

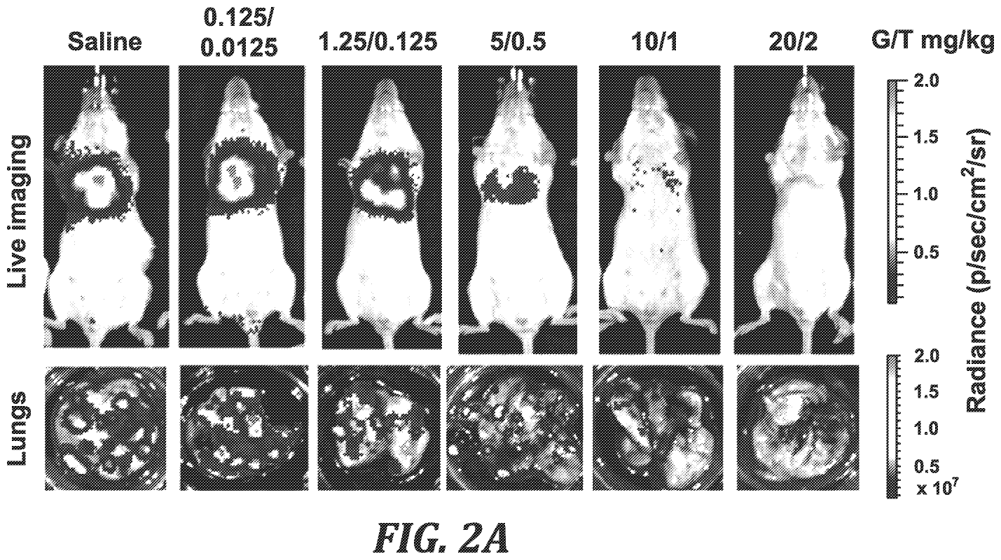

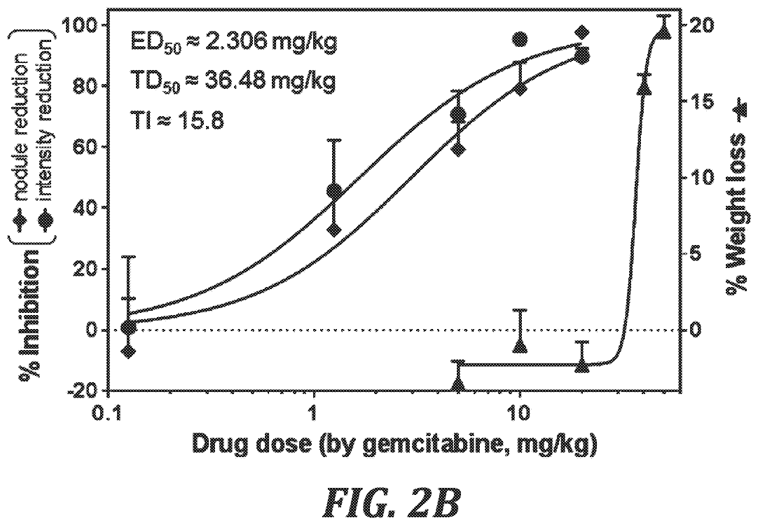

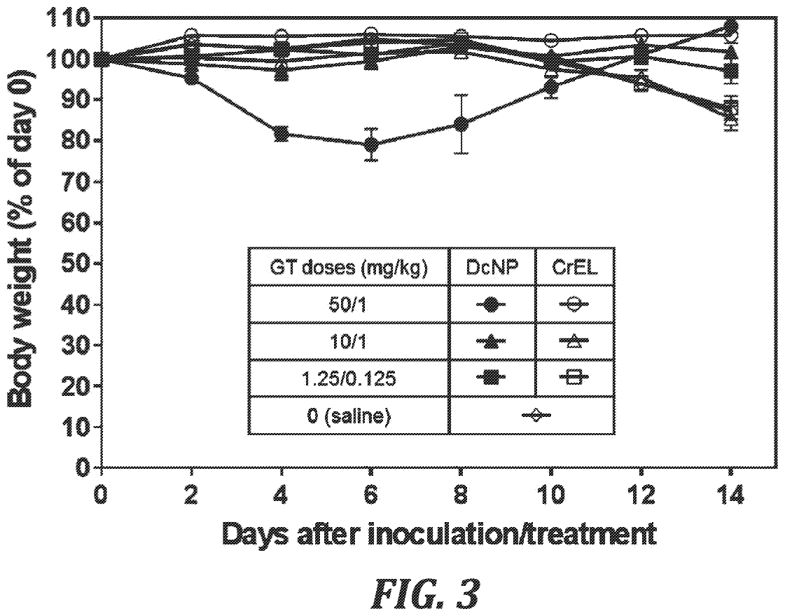

To confirm the initial results of the GT fixed-ratio combination in DcNP on 4T1 nodules in the lungs and establish preliminary dose-dependent effects, a dose-finding study was performed. Within the range of a single IV dose of DcNP carrying 0.125 to 50 mg/kg gemcitabine and 10/1 w/w paclitaxel (0.0125 to 5 mg/kg), the growth (based in 4T1-luc bioluminescence intensity) of tumor nodules was followed as therapeutic outcome measures. No clinical behavioral or hematological effects were notable for animals in all treatment groups for the 14-day study. However, a reproducible measure is the weight loss detected within 4-6 days after dosing (FIG. 3). Thus, this measure was used as a gross toxicity, likely similar to that observed with general GI toxicity associated in humans treated with gemcitabine. As shown in FIG. 2A, dose-dependent measures of tumor nodule count and tumor intensity were noted, and the trend of these two measures was a similarly graded response. At 20 mg/kg gemcitabine plus 2 mg/kg paclitaxel in DcNP, these two measures exhibited a 90-95% inhibition of 4T1-luc tumor burden, indicating a smaller dose requirement for cancer clearance compared to initial data presented in FIGS. 1A-1D. The same level of clearance was not achievable for the CrEL drug combination, even with 5-fold higher doses (i.e., 80-90% inhibition at 100/10 mg/kg GT). The 50% effective doses (ED₅₀s) for GT in DcNP fixed dose combinations were determined to be 1.655/0.1655 and 2.958/0.2958 mg/kg based on luminescence intensity and nodule count respectively (FIG. 2B). Based on the 20% weight loss (a maximum number allowable for experimental study) as a gross toxicology measure, the dose-dependent weight loss profile exhibited a much higher dose range and did not occur until 30/3 GT mg/kg dose. The dose-response curve for weight loss, referred to as a toxic dose (TD) was steeper and well-separated from the GT DcNP dose range that inhibited 4T1-luc tumor. The 50% toxic or TD₅₀ was determined to be 36.48/3.648 mg/kg GT. Using the mid-point of effective dose and toxic (weight-loss) doses, and the average therapeutic index, the ratio of toxicity-to-effective dose was estimated to be about 15.8 for GT DcNP. Taken together, dose-ranging studies indicate that the effective dose range was lower and well-separated from the toxicity range for GT combination by about 16 fold when given in DcNP dosage form. These data also confirmed that a single IV dose can clear a significant burden of invasive 4T1 with a sufficient margin of safety.

### Reproducibility of GT DcNP's Physicochemical and In Vivo Study Data

Due to inherent challenges in translating nanomedicine into clinical applications, it was important to validate the reproducibility of our GT DcNP regarding physicochemical properties and in vivo effectiveness. Due to high variance of the animal models, at least two independent effectiveness studies for each DcNP dose reported in FIGS. 2A and 2B was carried out. A two-way ANOVA analysis indicated that the difference between mean bioluminescence of replications for the same doses were insignificant but significant between difference doses (p=0.0854 for replication factor and p<0.0001 for dose factor). The results verified that the DcNPs produced in different batches exhibit similar characteristics—nearly identical mean particle size, drug loading and association efficiency, and more importantly, the ability to inhibit 4T1 metastasis in mice; thus, these results were reproducible and consistent.

With early detection and targeted therapies, breast cancer survival rates have increased, even as a cure remains elusive. When dealing with advanced metastatic disease, highly potent but toxic combination regimens such as GT are a limited option with dose-limiting toxicity as a significant barrier. Capitalizing on the drug combination nanoparticle (DcNP) platform's ability to stabilize water soluble gemcitabine and insoluble paclitaxel together into GT DcNP, it was found that the DcNP enhanced the plasma drug exposure at higher and longer lasting levels in mice. In a 4T1-luc metastatic mouse model, a single dose of GT DcNP was able to completely suppress 4T1 metastasis in the lung tissues. Dose-response studies also revealed the enhanced efficacy of GT drug combination and extended safety margin when given in GT DcNP dosage form.

For metastatic breast cancer treatment, combination therapy has been proven more effective than monotherapy. However—due to the disparate physicochemical properties—co-formulation for the targeted delivery of hydrophilic and hydrophobic drugs such as GT has been challenging. The disparate physical properties of the two drug combinations prevent them from associating together in a stable form. While liposome encapsulation of doxorubicin—via a complex manufacturing process removing unbound drug and precise remote loading—is available as Doxil, it is a single agent that must be combined with other agents expressing different pharmacokinetics, tissue distribution, and time courses for combination therapy. The benefit of liposomal doxorubicin is derived from enhanced tumor tissue accumulation through the neovasculature, which is formed at a later stage of tumor nodule development. A product with two-drugs encapsulated in liposomes called Vyxeos was recently approved by the FDA. Vyxeos contained two water soluble drugs cytarabine and daunorubicin. However, both drugs were subjected to labor intensive purification mentioned above (followed by lyophilization as a finished product) and only intended for treating leukemia, which exhibits significantly different cancer biology from metastatic breast cancer. Unlike liposome encapsulation, the DcNP platform was based on water-soluble (i.e., gemcitabine) and insoluble (i.e., paclitaxel) co-solubilized in a soft organic solvent (i.e., ethanol) together with lipid excipients serving as a bridge or glue. Removal of the solvent and rehydration allowed the formation of a stable drug-combination complex that can be broken down into GT DcNP particles at a size that is amenable for use as an injectable dosage form. Thus, this simplified process required no unbound drug separation, purification, or lyophilization, which could help with product scaling, reproducibility, and cost-saving.

The current human GT combination was given in a sequence with IV infusions of paclitaxel followed by gemcitabine (after 2-3 h) at doses of ˜1250/175 mg/m² GT, equivalent to ˜35/3.5 mg/kg. Sequential dosing of conventional GT was necessary both to improve tolerability and reduce toxicity. Both drugs were combined in the present Example while exhibiting sufficient safety in mouse models. The effective dose-range 10-50/1-5 mg/kg GT in mice was within the current range of human doses given in multiple cycles. Without wishing to be bound by theory, it is believed that this enhanced therapeutic index was likely through enhancement in differential drug distribution and pharmacokinetic profile. With two drugs in one intravenous injection, the DcNP formulation has prolonged the apparent elimination half-life of gemcitabine by more than 8× and enhanced its AUC by nearly 60×, higher than known previous achievements (see, e.g., Paolino D. et al., Cosco D. et al., Gemcitabine-loaded PEGylated unilamellar liposomes vs GEMZAR®: Biodistribution, pharmacokinetic features and in vivo antitumor activity. Journal of Controlled Release. 2010; 144(2):144-50; Zhang J. et al., Co-Delivery of Gemcitabine and Paclitaxel in cRGD-Modified Long Circulating Nanoparticles with Asymmetric Lipid Layers for Breast Cancer Treatment. Molecules. 2018; 23(11):2906, each of which is incorporated herein by reference in its entirety). Such enhancement can be due to association to DcNP, together with reduced paclitaxel clearance; plus potentially prevent exposure of DcNP bound gemcitabine inactivation by cytidine deaminase (to 2′,2′-difluorodeoxyuridine, or dFdU) in liver and cells. Regardless of pharmacokinetic and physiologic mechanisms, the DcNP formulation has enhanced the GT pharmacokinetic and pharmacodynamic profile resulting in an ˜10-fold lower GT dose needed to inhibit metastatic cancer with a safety margin (TI of 15.8).

The therapeutic effects mediated by DcNP on GT were evaluated in 4T1 inoculated systematically to produce the lung metastasis model. This model was immunocompetent and relevant to human disease, where immune contribution was important. A genomic profiling study revealed a high consistency between lung metastases from orthotopic (mammary fat pad) and IV inoculation models, demonstrating that this approach mimicked the spread of metastatic breast cancer cells from the primary tumor site. This model was also clinically relevant to human disease due to reported spontaneous 4T1 metastasis to the lungs, brain, and bones in mice with functional immune systems. Models generated with murine originated 4T1 cells have proven useful in metastasis disease and interventional studies and were used extensively in discovering immuno- and chemotherapeutics targeting metastatic breast cancer.

Limited by safety concerns and the short half-lives of most current chemotherapeutic drugs, repeated dosing regimens were typically used in humans. Single agent drug resistance, and cumulative drug toxicity for some drugs could limit treatment options for metastatic diseases. In mouse model of this study, cancer cells progressed into the lungs from the blood to cause mortality in only ˜14 days. In this time window, the highest achievable single dose of the CrEL drug combination (limited by paclitaxel solubility) inhibited the process marginally. For 80˜90% inhibition of lung metastasis of 4T1 within a similar timeframe, multiple dosing was required in previous studies (see, e.g., Cao H. et al., Hydrophobic interaction mediating self-assembled nanoparticles of succinobucol suppress lung metastasis of breast cancer by inhibition of VCAM-1 expression. Journal of Controlled Release. 2015; 205:162-71; Cao H. et al., Liposomes Coated with Isolated Macrophage Membrane Can Target Lung Metastasis of Breast Cancer. ACS Nano. 2016; 10(8):7738-48; Dan Z. et al., A pH-Responsive Host-guest Nanosystem Loading Succinobucol Suppresses Lung Metastasis of Breast Cancer. Theranostics. 2016; 6(3):435-45; and Chen Q, Ross AC. All-trans-retinoic acid and the glycolipid α-galactosylceramide combined reduce breast tumor growth and lung metastasis in a 4T1 murine breast tumor model. Nutr Cancer. 2012; 64(8):1219-27, each of which is incorporated herein in its entirety). Even with more sophisticated nanoparticles with target-activated drug delivery (e.g., legumain), none of the reports were able to demonstrate clearing of the 4T1 lung metastasis in a single dose. See, e.g., He X. et al., Inflammatory Monocytes Loading Protease-Sensitive Nanoparticles Enable Lung Metastasis Targeting and Intelligent Drug Release for Anti-Metastasis Therapy. Nano Letters. 2017; 17(9):5546-54, incorporated herein by reference in its entirety). In contrast, GT DcNP inhibited 100% of the lung metastasis with no detectable cancer in vivo and ex vivo with only one single IV injection (FIGS. 1A-1D and FIGS. 2A and 2B).

The ability of DcNP to enhance GT combination could enhance cancer chemotherapy. As lymphatic cancer invasion was believed to be an early site for metastasis, DcNP loaded drugs can provide additional benefits. After subcutaneous administration and during the first passage, DcNP particles are shown to track preferentially into the lymph, but not blood capillaries, as observed with a near-infrared fluorophore (indocyanine green) tagged DcNP in mice. In addition, DcNP has enabled the maintenance of cellular drug levels in lymph node mononuclear cells (above plasma drug levels) for over 2-4 weeks in non-human primates (NHP). With alternative subcutaneous routes of GT DcNP administration, it is believed that drugs could be localized in the lymphatic system and stand ready to block the lymphatic metastasis pathway.

Thus, the present Example describes a simple, stable, and scalable GT DcNP dosage suitable for IV administration that transforms short-acting gemcitabine into a long-acting variation. The enhanced GT pharmacokinetic profile provided by DcNP dosage form paralleled the GT effect against 4T1 metastatic breast cancer. A single GT DcNP injection completely inhibited lung metastasis in mice at levels that cannot be achieved with a CrEL dosage form at equal or higher doses. The enhanced dose-response was observed with a significant margin of safety. With the flexibility of the DcNP platform and impact on GT pharmacokinetics, pharmacodynamics, and safety windows, it is believed that this approach could be generally applicable for use with other drug combinations intended to treat a number of cancer types, especially for the metastatic disease stage. The ability to transform current short-acting drugs into long-acting forms with preferential uptake could also accelerate clinical translation of the drug combinations in DcNP dosage form.

### Example 2. Long-Acting Drug Combination Nanoparticles Composed of Gemcitabine and Paclitaxel Enhance Localization of Both Drugs in Metastatic Breast Cancer Nodules

An optimized GT DcNP composition (d=59.2 nm±9.2 nm) was found to be suitable for IV formulation. Plasma exposure of G and T were enhanced 61-fold and 3.8-fold when given in DcNP form compared to the conventional formulation, respectively. Mechanism based pharmacokinetic modeling and simulation show that both G and T remain highly associated to DcNPs in vivo (G: 98%, T:75%). GT DcNPs have minimal distribution to healthy organs with selective distribution and retention in tumor burdened tissue. Tumor bearing lungs had a 5-fold higher tissue-to-plasma ratio of gemcitabine in GT DcNPs compared to healthy lungs.

### Abbreviations

DcNP: Drug combination nanoparticle

CrEL: Cremophor El suspension

AUC: Area under the curve

C₀: Concentration at time 0

T_{1/2}: Half-life

Dose/AUC: Apparent clearance

Vₛₛ: Volume of distribution at steady state

MRT: Mean residence time

AUMC: Area under the moment curve

GT: Gemcitabine and paclitaxel combination

G: Gemcitabine

T: Paclitaxel

MBPK: Mechanism-based pharmacokinetic model

K: rate constant

dFdU: 2′,2′-difluoro-deoxyuridine

CDA: Cytidine deaminase

dCK: deoxycytidine kinase

The goal of this study is to determine whether water soluble G and water insoluble T can be stably assembled together in DcNP form and allow synchronization of both drugs in metastatic breast cancer burdened tissue. GT was stabilized in DcNP form and transformed GT from a current short-acting combination therapy into a long-acting combination therapy in target tissues and cells. In addition, pharmacokinetic modeling and simulations were used to distinguish DcNP associated and dissociated fractions of GT in plasma. By doing so, how the fraction of drug association to DcNP in vivo impacts the overall pharmacokinetics and exposure of GT when formulated together in DcNP dosage form can be investigated.

### Materials and Methods

G (>99%) and T (>99.5%) were purchased from LC Laboratories (Woburn, Mass.). 1,2-distearoyl-sn-glycero-3-phosphocholine (DSPC) and 1,2-distearoyl-sn-glycero-3-phosphoethanolamine-N-[amino (polyethylene glycol)-2000] (ammonium salt) (DSPE-PEG₂₀₀₀) (GMP grade) were purchased from Cordon Pharma (Liestal, Switzerland). Anhydrous ethanol was purchased from Decon Pharmaceuticals (King of Prussia, Pa.). All other reagents used were of analytical grade or higher.

### Preparation and Characterization of Gemcitabine and Paclitaxel (GT) Drug Combination Particles

T (0.7 mg/mL) and G (7 mg/mL) were solubilized together in hot ethanol (60° C.) with DSPC (25 mg/mL) and mPEG₂₀₀₀-DSPE (10 mg/mL) in a round bottom flask. The total concentration of solutes (drugs+excipients) in ethanol was 5% w/v. Solvent was removed by rotary evaporation followed by vacuum desiccation. The dry film was removed from the round bottom flask and triturated to achieve a uniform dry powder. Dry powder was rehydrated in 0.45% NaCl with 20 mM NaHCO₃ buffer at 70° C. and a pH of 7.4 to achieve a nominal concentration of 100 mM total lipids. Particle size reduction was achieved through bath sonication (Avanti Polar Lipids, Inc. Alabaster, Ala.) (5 min on, 5 min off, 3 cycles). Particle size and zeta potential were determined by photon correlation spectroscopy using a NICOMP 380 ZLS (Particle Sizing Systems, Santa Barbara, Calif.). The pH of the DcNP suspension was measured using MQuant pH-indicator test strips (SupelcolSigma Aldrich, St. Louis, Mo.). The osmolarity of GT DcNPs was measured using a Vapro osmometer (Wescor Inc, Logan, Utah).

The morphology of GT DcNPs was investigated compared to a liposome control using transmission electron microscopy (TEM) with negative staining. Liposome controls were formed by dissolving egg L-α-phosphatidylcholine (EPC) (Avanti polar lipids, Inc. Alabaster, Ala.) in chloroform. Chloroform was removed via rotary evaporation and the dry lipid film was rehydrated in normal saline. The rehydrated lipid film was then extruded through 100 nm pores to yield a liposome suspension. Sample suspensions containing either GT DcNPs or liposomes were transferred onto a TEM grid (copper grid, 300-mesh, coated with carbon and Formvar film). Particles from the sample suspensions were allowed to settle onto the grid and excess suspension was removed by filter paper after 5 min. The grid was then stained with 4 μL 5% uranyl acetate. After one minute, excess staining solution was removed by filter paper and the grid was air-dried. All images were acquired on a Tecnai G2 F20 electron microscope (FEI, Hillsboro, Oreg.) operating at 200 kV.

Drug association efficiency (AE %) was determined by dialyzing 100 μl of the DcNP suspension (6-8 k MWCO) against 1000×volume (100 mL, pH=7.4) of bicarbonate buffered saline for 4 hours at room temperature. Drugs were extracted by acetonitrile and drug concentrations in pre and post-dialysis DcNP suspensions were measured with a Shimadzu HPLC-UV system (Kyoto, Japan). Chromatographic separation was achieved using a Kinetex C18 column (100 Å, 5 μm, 4.6 mm×100 mm) (Phenomenex, Torrance, Calif.). The flow rate was set to 1.0 mL/min with a 10 μl sample injection volume. The mobile phase for separation consisted of pump A (Acetonitrile) and B (10 mM Ammonium Acetate in water). The gradient program used was as follows: pump B was set to 40%, and increased to 100% over 5 minutes. The wavelength for detection of gemcitabine and paclitaxel was 254 nm. AE % was calculated as the ratio of pre- over post-dialysis concentrations of G or T.

### Preparation of Gemcitabine and Paclitaxel (GT) Combination in Cremophor EL Suspension (CrEL)

To prepare an equivalent GT drug combination for use as a control formulation, T was first dissolved in ethanol (20 mg/mL). To make a stable suspension, the 20 mg/mL T was diluted with Cremophor EL [1:1, (v/v)] (Sigma-Aldrich, St. Louis, Mo.). The resultant T in suspension was further diluted 10-fold with PBS containing pre-dissolved G (hydrochloride salt, 12.65 mg/mL). The final concentrations of drug combination in suspension were 10 mg/ml G and 1 mg/ml T. The control drug combination in CrEL suspension was used in animal studies within the same day of preparation due to instability.

### Pharmacokinetic Study of Gemcitabine and Paclitaxel (GT) in DcNPs Compared to CrEL

Animal studies were conducted in accordance with the University of Washington Institute of Animal Care and Use Committee (IACUC) approved protocol number 2372-06 and federal guidelines. Five- or six-week old female BALB/c mice were purchased from The Jackson Laboratory (Bar Harbor, Me.) and housed in an animal research facility for at least one week before use. Mice were approximately 8 to 9 weeks old for the pharmacokinetic studies. Mice were administered GT either as DcNP or CrEL suspensions intravenously by tail vein injection at 50/5 mg/kg (G/T) in a 100 μl bolus volume (G ˜10 mg/mL, T ˜1 mg/mL). Blood was collected through retro-orbital bleeding at 5, 60, 120, 360, 1440 (24 hour), and 2880 min (48 hour) for DcNP and at 5, 60, 120, 360 min for the GT CrEL formulation. Each mouse represented a single biological replicate and 3 mice were used to estimate the mean plasma concentration time course of G and T at each time point. Necropsies were performed on each animal to harvest respective tissues for tissue distribution studies.

Drug Extraction from Plasma and Tissues

Liquid-liquid extraction was used to extract G, 2′,2′-difluoro-deoxyuridine (dFdU), and T from plasma or tissue homogenates. Briefly, 50 μL of sample was transferred into 1.5 mL tubes with or without dilution by blank matrix to an appropriate concentration range. Samples were spiked with internal standards followed by the addition of 9 volumes of acetonitrile (450 μL). Samples were then vortexed for 6 minutes and centrifuged at 4° C. for 15 minutes at 14000 rpm. The supernatant was removed and dried under nitrogen at 40° C. The dried samples were reconstituted to 50 μL containing 20% methanol and 80% water.

Quantification of Gemcitabine, Paclitaxel and dFdU by LC-MS/MS

Drugs extracted from biological matrices such as plasma or tissue homogenates were quantified by a Shimadzu HPLC system coupled to a 3200 QTRAP mass spectrometer (Applied Biosystems, Grand Island, N.Y.). The HPLC system consisted of two Shimadzu LC-20A pumps, a DGU-20A5 degasser, and a Shimadzu SIL-20AC HT autosampler. The mass spectrometer was equipped with an electrospray ionization (ESI) TurbolonSpray source. The system was operated with Analyst software, version 1.5.2 (ABSciex, Framingham, Mass.). Chromatographic separation of G and T was achieved using a Synergi column (100×2.0 mm; 4-μm particle size) (Phenomenex, Torrance, Calif.) with an inline C8 guard column (4.0×2.0 mm) also from Phenomenex. The flow rate was set to 0.5 mL/min with a 5 μl sample injection volume. The mobile phase for separation consisted of pump A (20 mM Ammonium Acetate in water) and B (Reagent Alcohol). The gradient program used was as follows: pump B was maintained at 20% for 1.0 minute, then increased to 97% at 2.0 minutes, held at 97% until 3.0 minutes, ramped to 3% by 4.0 minutes and held until 5.5 minutes. The needle was washed with isopropanol after each injection. Analytes were monitored using multiple-reaction monitoring (MRM) for positive ions. The following ion transitions were monitored: gemcitabine, m/z 264.066→112.000; dFdU, m/z 265.084→413.200; paclitaxel, m/z 854.266→286.200; a stable labeled isotope of gemcitabine (C₈C¹³ H₁₂ClF₂N¹⁵N₂O₄) (m/z 267.067→115.100) was used as an internal standard for gemcitabine and dFdU; docetaxel (m/z 830.312→549.3) was used as an internal standard for paclitaxel.

Estimating the Maximum Dissociated Fraction of Gemcitabine and Paclitaxel In Vivo when Administered as a DcNP

To estimate a maximum dissociated fraction (f_{diss. max}) of GT from DcNPs in vivo, a non-compartmental approach based on the plasma AUCs of equi-molar injections of DcNP and CrEL formulations was first utilized:

$${f}_{diss . max} = \frac{A U {C}_{0 \to ∞ , CrEL}}{A U {C}_{0 \to ∞ , DcNP}} \qquad [ Equation 1 ]$$

In Eq. 1, it is assumed that systemic clearance of G or T administered as DcNPs only occurs after drug dissociates from the particles, and dissociated drug has the same clearance pathways as the free drug control (CrEL). This non compartmental approach also assumes that drug clearance occurs solely in the central compartment. For the two drugs of interest, T is mainly metabolized in the liver by CYP3A4 and CYP2C8 but G is metabolized by ubiquitous cytidine deaminase (CDA). To understand the impact of the ubiquitous metabolism of G on its pharmacokinetic profile, the plasma concentration of the primary metabolite of G, dFdU, was used as a biomarker for dissociated drug. For Eq. 2, it is assumed that G conversion to dFdU exhibits linear kinetics. The equation below then provides an estimate of the fraction of the G dose metabolized to dFdU. If the calculated fraction metabolized (f_{diss.G→dFdU}) from Eq. 2 equals the f_{diss. max} from Eq. 1, it can be surmised that once G is released from DcNPs at all locations in the body it exchanges readily with the circulating G and subsequently undergoes metabolism (i.e., metabolism of all dissociated drug occurs in the central compartment). However, if some or all of the drug released from DcNPs in peripheral tissue is immediately metabolized to dFdU without exchanging with G in circulation, the f_{diss.G→dFdU} estimates from Eq. 2 would be expected to exceed the f_{diss. max} from Eq. 1 (i.e., dissociated drug is metabolized in both the central and peripheral compartments).

$$\frac{{GAUC}_{0 \to ∞ , CrEl}}{dFdU {AUC}_{0 \to ∞ , CrEl}} = \frac{{f}_{diss . G \to dFdU} \cdot G {AUC}_{0 \to ∞ , DcNP}}{dFdU {AUC}_{0 \to ∞ , DcNP}} \qquad [ Equation 2 ]$$

Which can then be simplified to:

$${f}_{diss . G \to dFdU} = \frac{G {AUC}_{0 \to ∞ , CrEl}}{dFdU {AUC}_{0 \to ∞ , CrEl}} \cdot \frac{dFdU {AUC}_{0 \to ∞ , DcNP}}{G {AUC}_{0 \to ∞ , DcNP}}$$

G AUC_{0→∞,CrEL} is the plasma AUC of G given in free drug form. G AUC_{0→∞,DcNP} is the total AUCs of associated and dissociated G given in DcNP form. dFdU AUC_{0→∞, CrEL} is the AUC of dFdU formed after administration of free G. dFdU AUC_{0→∞,DcNP} is the AUC of dFdU after dosing G in DcNP, which reflects the amount of dissociated G released from DcNPs and available for metabolic conversion. f_{diss.G→dFdU} represents the fraction of DcNP-dose that is released as free drug and available for conversion to dFdU by CDA.

The assumptions underlying Eq. 2 are as follows: [1] Dissociated (free) G, but not associated G, is readily available for metabolism by CDA. [2] Dissociated G is rapidly and extensively metabolized to dFdU by CDA. [3] The free fraction of dissociated Gin plasma and tissue is independent of drug concentration (i.e., protein binding does not change with G concentration). A detailed pharmacokinetic model for the associated and dissociated species will be presented next to simulate the in vivo association of both drugs.

### Mechanism-Based Pharmacokinetic Modeling (MBPK) to Estimate DcNP Associated and Dissociated Fractions of GT

To further evaluate the strong in vivo association of both G and T to DcNPs, we have utilized a mechanism-based pharmacokinetic model (MBPK) to simulate the plasma time course of the associated and dissociated drugs.

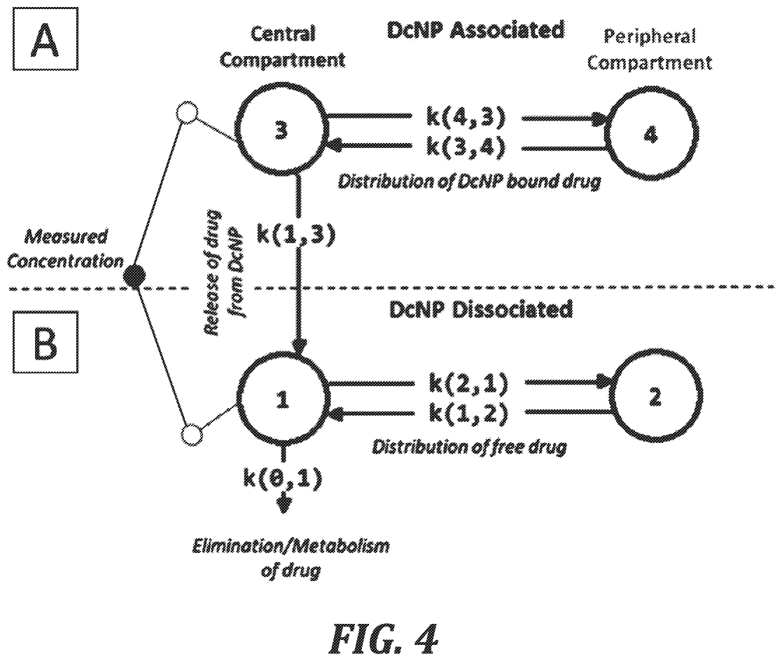

Briefly, the model adopted here for GT is composed of two sub-models representing DcNP-associated drug and dissociated drug species. Each sub-model has a central compartment and a peripheral compartment. A visual representation of the model is presented in FIG. 4. The observed plasma concentration is the sum of DcNP-associated and dissociated species since they cannot be measured separately. The dissociated drug pharmacokinetic parameters, k_{1,2}, k_{2,1} and k_{0,1}, were determined by fitting the dissociated drug sub-model to the observed plasma concentration-time data for G and T after injection of CrEL (free-drug) suspension. These parameters were then fixed and the linked sub-models were fitted to the observed total concentrations for G and T obtained after injection of the DcNP-dose. The model reasonably assumed that the pharmacokinetics of drugs released from particles will be the same as that of the free-drug (CrEL) control. It was assumed that G and T are released from the particle at independent rates (k_{1,3}) in the central compartment and only dissociated drugs are subject to clearance from the system (k_{0,1}). The model input was the dose, which was assumed to be 100% associated for T based on the high in vitro association (95%) and corroborated by Eq. 1 (>75%, See results). Although G in vitro association was 9%, it was assumed that G was also 100% associated per the in vivo results from Eq. 1 (>98%, See results). In fact, when 9% G association (based on in vitro dialysis under sink conditions) was assigned to the DcNP-dose, the model fit did not converge due to gross underprediction of the observed plasma G concentration-time data. The peripheral compartment features purely drug exchange between plasma and a group of slowly equilibrating tissues (k_{4,3}, k_{3,4}). In other words, the DcNP-associated drug is cleared only via its release from the particles in the central compartment. Thus, Eq.1 would provide a reasonable boundary estimate for the dissociated fraction after DcNP-associated drug injection and should be close to the estimate from the modeling. The model predictions fit the observed total plasma drug concentration-time data well (R²>0.9 for both G and T). MBPK modeling and parameter estimations were performed using SAAM II v2.3.

### Establishment of Metastatic Nodules in Lungs

Female BALB/c mice were injected with 2×10⁵ 4 T1 metastatic breast cancer cells that express luciferase as a marker intravenously by tail vein on day 0. Stable expression of luciferase in these cells allows for bioluminescent monitoring of tumor growth and metastasis. Over a 14-day period, mouse behavior and health conditions were monitored daily and body weight measurements were taken every 2 days. On day 14, mice were administered 150 mg/kg D-luciferin through intraperitoneal injections 10 to 15 min before in vivo imaging to confirm establishment of tumor nodules in lungs. The bioluminescence imaging was acquired through a XENOGEN IVIS 200 imaging system (PerkinElmer, Waltham, Mass.). The bioluminescence imaging parameters for live mice were set as follows: field of view, 24; excitation filter, closed; emission filter, open; exposure time, 180 sec; binning factor, 4; f/stop, 2. Bioluminescence intensity from mice were integrated using Live Image software (PerkinElmer, Waltham, Mass.). Mice were then intravenously administered GT in DcNPs at a dose of 50 mg/kg G and 5 mg/kg T or the same dose of drug in control suspension (CrEL) and sacrificed at fixed time points.

### Statistical Analysis

Statistical analysis was performed using GraphPad Prism 7.04 (GraphPad Software Inc., San Diego, Calif.). Statistical comparisons were performed using 2-sided t-tests with Welch's correction for unequal variances. Significance probability α was set at 0.05. Pharmacokinetic parameters from non-compartmental analysis were calculated using the trapezoidal rule and relevant pharmacokinetic equations shown in Table 3.

### Results

### Preparation and Characterization of Injectable GT Combination in Drug Combination Nanoparticle Form

To determine whether two chemically distinct cancer drugs of interest (G, log P=−1.4 and T, log P=3) can be formulated together in a single particle suitable for IV injection, several lipid excipient combinations that exhibit amphipathic properties were investigated. Hydrophobic T may interact with the acyl chains of phospholipids while the hydrophilic G may interact with the pegylated periphery of lipids when drugs and lipid excipients are assembled together. The two lipid excipients in the composition were able to stabilize GT in a DcNP in suspension. After formulation and composition optimization, a DcNP composition with 10:1 (w/w) G-to-T ratio, containing two lipid excipients (DSPC, DSPE-PEG₂₀₀₀, 9:1 m/m) was able to produce a stable DcNP suspension with a total drug to total lipid ratio of 1:12 (w/w) for use as an IV injectable dosage form. With the intent to streamline scale-up processes, the preparation procedure was designed to minimize processes such as removal of unbound drug. This process appears to be robust and reproducible with consistent AE % of drugs (see below). Product characteristics including degree of drug association (AE %) to DcNP in suspension were evaluated under sink conditions and batch to batch variability in size, drug concentration and association efficiency. These data are presented in Table 2. The pH, zeta potential, osmolarity and morphology of GT DcNPs were also characterized.

TABLE 2
Characterization and batch-to-batch variability of GT DcNPs.

| | | | | [Drug Cone] | |
|---|---|---|---|---|---|
| | | AE %^{b} | | (mg/mL) | |
| Batch | Size (nm)^{a} | PTX | GEM | PTX | GEM |
| 1 | 49.2 ± 35 | 95 | 7 | 1 | 13.9 |
| 2 | 59.9 ± 44 | 97 | 9 | 1.2 | 13.9 |
| 3 | 69.7 ± 47 | 97 | 10 | 1.3 | 15.3 |
| 4 | 68.7 ± 46 | 97 | 10 | 1.1 | 14.5 |
| 5 | 48.5 ± 30 | 92 | 8 | 0.7 | 14 |
| Average | 59.2 | 96 | 9 | 1.1 | 14.3 |
| S.D | 9.2 | 2 | 1 | 0.2 | 0.5 |

^{a}Mean particle diameter was determined by photon correlation spectroscopy and presented as the mean ± standard deviation
^{b}Association efficiency of gemcitabine (Gem) and paclitaxel (PTX) was determined by dialysis under sink conditions as described in Materials and Methods.

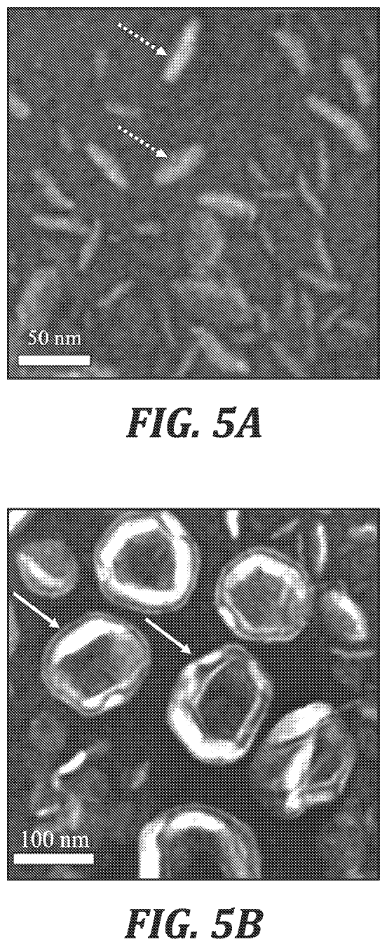

A total of 5 batches were tested for formulation process and quality with respect to reproducibility. The mean particle size for all 5 batches was 59.2±9.2 nm and suitable for IV dosing. The degree of drug association to DcNP was measured and expressed as AE % measured under sink conditions in buffered normal saline. For G, the average AE % for 5 batches was 9±1; for T, the average AE % for 5 batches was 96±2. The pH and osmolarity of GT DcNPs in suspension were consistent across batches and measured to be pH=8.0 and 355 milliosmoles, respectively. Both of these parameters fall within acceptable boundaries for IV injectable products. The average zeta potential of GT DcNPs was measured to be −16.4 mV. The morphology of GT DcNPs was investigated using transmission electron microscopy (TEM). GT DcNPs have a distinct, discoid-like shape (FIG. 5A) with no apparent bilayer structure. In contrast, the conventional liposome controls (FIG. 5B) are observed to have lipid vesicles with enclosed bilayer membranes.

Collectively, these data indicate that GT DcNPs are suitable for injectable dosage forms and can be prepared in a reproducible manner. The final composition for GT DcNPs had nominal concentrations of 16 mg/ml G and 1.6 mg/ml T. The GT DcNP injectable dosage form was subsequently diluted to 10 mg/mL and 1 mg/mL for use in pharmacokinetic studies in mice.

### Effect of DcNP Formulation on Gemcitabine and Paclitaxel Plasma Time Course and Pharmacokinetics

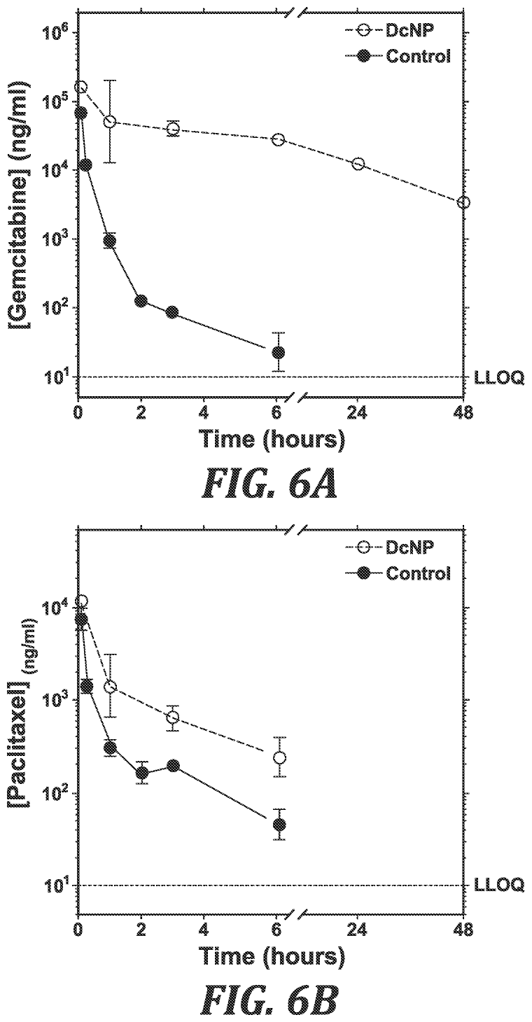

The effects of DcNP formulation on G and T in vivo was then investigated. Due to the limited solubility of T, a Cremophor EL (referred to as CrEL) suspension was used to stabilize the GT control dosage form for IV administration. Although this CrEL-based micellar formulation may not fully represent a free and soluble T control, it does represent the clinical dosage form of T (Taxol). Thus, intravenously administered G and T (50/5 mg/kg G/T) to mice was compared in either DcNP or control CrEL-solubilized form. The total drug concentrations of G and T were determined in plasma at indicated time points. These data are presented as a plasma concentration time course comparison in FIGS. 6A and 6B. Pharmacokinetic parameter estimates are presented in Table 3.

As shown in FIGS. 6A and 6B, the plasma drug concentration time course of G and T were substantially different when administered as DcNPs in comparison to CrEL control dosage form. Based on their respective in vitro AE % (9% for G and 95% for T under sink conditions), a greater difference in exposure for T than G was expected. However, to the contrary, much greater enhancement in plasma concentrations of G than T when comparing DcNP to that of the CrEL control was observed (FIGS. 6A and 6B). For example, 3 hours after DcNP dosing, mean plasma G concentration was 470 times greater than at the same time point for mice treated with the control CrEL (41,065 ng/mL vs 87.28 ng/mL at 3 hours, p<0.05). For T, there was also a higher plasma drug concentration in those treated with DcNP compared to the CrEL control; however, the formulation effect was more modest. At 3-hours, the plasma T concentration for DcNP was 3.3× greater than CrEL (642.9 ng/mL vs 193.6 ng/mL at 3-hour, p<0.05). By 24 hours, the plasma G and T concentrations in mice treated with the CrEL control formulation fell below the detection limit (LLOD). In contrast, for the test group treated with DcNP dosage form, persistent G concentrations in plasma were detected for the entire 48-hour study in mice administered DcNPs (FIG. 6A). No T concentrations were detected in plasma after 6 hours in mice likely due to its much lower dose (50 mg vs 5 mg/kg G to T ratio in DcNP formulation) (FIG. 6B).

The plasma drug concentration time course was further analyzed using non-compartmental analysis. The pharmacokinetic parameters are presented in Table 3. The total exposure or area under the curve (AUC) of G is increased by 61-fold when administered as a GT DcNP compared to the control CrEL suspension (56218.6 vs 920.8 μg*min/mL). Since both DcNP and control groups received the same dose of G (50 mg/kg), the apparent clearance (represented by dose/AUC) for the DcNP cohort decreased reflective of the increase in exposure (1.1 vs. 65.2 mL/hour). No major change is observed in the concentration of G in plasma at time 0 (C₀) after administration in DcNP or control suspension (181.4 versus 165.1 μg/mL). The apparent half-life (t_{1/2} app) increased 8.6-fold when administered in DcNP form compared to the control suspension (13.7 vs 1.6 hours), reflecting a change in the long-acting plasma time-course that extended the apparent terminal slope of G. There was a limited reduction in volume of distribution at steady state for G in DcNP and free forms (12.1 mL vs 16.6 mL). Mean residence time for G, or the average time G molecules stay in the body, increases when given in DcNP form (11.3 vs. 0.25 hours). Considering the relative change in exposure from G administered in DcNP form versus free form, it is unlikely that G is only 9% associated as indicated by in vitro dialysis under sink conditions. Alternatively, assuming that systemic clearance of G administered in DcNP form can only occur after dissociation and the clearance mechanisms do not differ between free and DcNP form, then Eq. 1 can provide an in vivo estimate of the maximum dissociated fraction (f_{diss. max}) of G. This estimate is calculated to be 1.6% and suggests that G is mostly associated in vivo.

For T, the total exposure or AUC is increased by 3.8-fold when administered as a DcNP versus a CrEL control suspension. Both groups were administered the same dose of T (5 mg/kg) and apparent clearance decreased reflective of the increase in exposure (10.2 vs 38.3 mL/hour). Interestingly, the volume of distribution at steady state changed in concert with clearance when given in DcNP or control form (10 mL vs 35.6 mL) and to a greater degree than G. No major change is observed in the initial concentration of T in plasma when administered as either the CrEL suspension or DcNP. In contrast to G, no change is observed in the apparent half-life or mean residence time of T when given in DcNP or control suspension (2.0 vs 1.8 hours; 1.0 vs 1.0 hours, respectively) (Table 3). Using the same assumption stated above, the in vivo association of T can be estimated by Eq. 1. The f_{diss. max} of T is calculated to be 26.6%.

The changes to the in vivo behavior of GT administered as a DcNP compared to their conventionally solubilized control suggests that DcNPs are reasonably stable in vivo. If DcNPs degraded rapidly in plasma after IV administration, the drug would be expected to release from the particles and behave like the CrEL control suspension. Instead, a remarkable enhancement in G circulation in plasma for up to 48 hours and a lesser but notable enhancement with T were observed. Collectively, these data suggest that large fractions of both G and T remain associated to DcNPs after IV administration. This effect is surprisingly more remarkable for water soluble G, which was initially predicted to readily dissociate in blood according to in vitro predictions in buffer.

Effect of DcNP on Gemcitabine Metabolism to dFdU and Estimation of Dissociated Drug

To further investigate the discrepancy between low in vitro G association and the much higher than anticipated overall drug exposure in mice attributed to DcNP (FIGS. 6A and 6B and Table 3), the metabolic conversion of G to 2′,2′-difluorodeoxyuridine (dFdU) in cells and tissues was evaluated. In this instance, assuming that when G is bound to DcNPs, it is not accessible to CDA, the primary metabolizing enzyme of G that is present in plasma and peripheral tissues. Due to the ubiquitous expression of the CDA enzyme and its ability to convert G to dFdU in peripheral tissues, dissociated drug that undergoes immediate metabolism in the periphery is not directly accounted for using Eq. 1 (which is based on plasma G levels). With these assumptions, this primary metabolite of G (dFdU) was utilized as a marker for the fraction of G that dissociates from DcNPs, which subsequently undergoes metabolism in all tissues (following free G to dFdU conversion kinetics) as per Eq. 2. In the studies with IV injections, it was assumed that G in the control CrEL formulation is 100% freely soluble and available to CDA for conversion to dFdU in the body. The elimination of dFdU from the body occurs primarily through the kidneys and all dFdU formed in the body (peripheral tissues) would readily return to the central compartment for renal elimination. Under these conditions, when G is stably associated with DcNPs in the body, G is not available for conversion to dFdU. When G dissociates from the particle in either the central or peripheral compartment, it is then free to interact with CDA and undergo metabolism to dFdU.

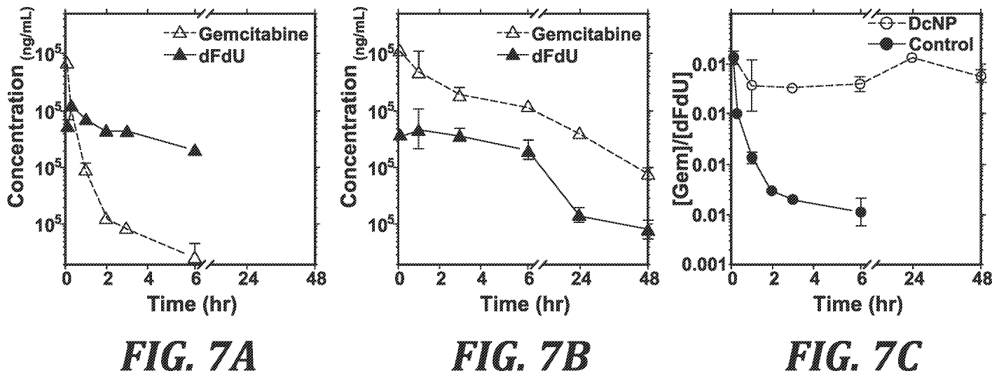

The results in FIG. 7A show the time course of G and dFdU after IV administration of GT in the CrEL control dosage form. The plasma concentrations of the primary metabolite (dFdU) rose rapidly and reached concentrations equivalent to G within 15 minutes (11836.7 ng/mL and 12023.5 ng/mL, respectively) and in 3 hours the concentration of dFdU in plasma was 50×times higher than G (4343.2 ng/mL vs 87.3 ng/mL). Over time, this gap became larger with the G to dFdU ratio falling to 0.01 in 6 hours. In contrast, when G was given as a GT DcNP dosage form the plasma concentration of dFdU did not reach the G levels throughout 48 hours of study (FIG. 7B). At 3 hours, plasma dFdU is 3.4×lower than G. The variation in G/dFdU ratios over time can be seen in FIG. 7C with open circles representing the CrEL control and closed circles representing DcNPs. Based on these data (FIGS. 7A-7C) and Eq. 2, the estimated fraction of dissociated G accessible for metabolism (f_{diss.G→dFduU}) is calculated to be 8%. In other words, 92% of G in DcNP dosage form is not accessible for dFdU conversion after IV GT-DcNP administration. The higher dissociated fraction of G derived from dFdU analysis is higher than the 1.6% estimate provided by Eq. 1 and suggests that peripheral metabolism competes with the redistribution of dissociated drug back into systemic circulation. Nevertheless, both estimates collectively point to the majority of G (>92%) remaining associated to DcNPs in vivo and being likely inaccessible by CDA for metabolic conversion.

These data are consistent with the initial premise that GT DcNP particle are sufficiently stable in vivo, and that G association to DcNPs is high throughout the course of the pharmacokinetic study. Additionally, the plasma time course of G and dFdU show that dFdU kinetics are elimination rate limited when given in free form and changes to formation-rate limited (or release-rate limited) kinetics when administered in DcNP form. DcNPs enabling a shift to release-rate limited kinetics may essentially act as an extended infusion of G and may be beneficial for therapeutic effect.

### Mechanism-Based Pharmacokinetic Simulations of DcNP-Associated and Dissociated Drug Time-Courses

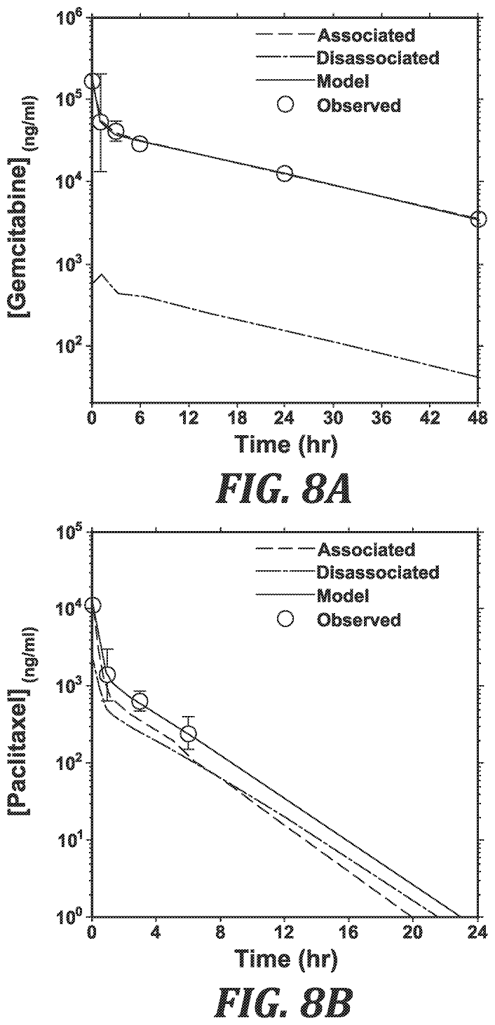

To further understand the effect of DcNP on the pharmacokinetic behavior of GT, a mechanism-based pharmacokinetic model (MBPK) was used to simulate the time course of DcNP-associated and dissociated drugs in plasma. The MBPK model adapted for GT-DcNPs is based on a validated MBPK model developed for long-acting HIV drug combination nanoparticles tested in non-human primates. Experimentally, the total drug concentrations in plasma (i.e., DcNP-associated plus dissociated) can be measured, but DcNP-associated and dissociated drugs could not be distinguished. Due to these limitations, direct comparisons of the pharmacokinetics of GT when administered in DcNP or CrEL control form are difficult to interpret. The MBPK model presented in FIG. 4 utilizes data from both DcNP and CrEL control formulation treated animals to estimate plasma concentrations of DcNP-associated and dissociated drug species over time and their respective time-averaged fractions in vivo. After intravenous administration as a DcNP, GT can theoretically exist in at least two species: DcNP-associated drug and dissociated drug. The latter can be reasonably assumed to distribute and be cleared as free G and T after administration of CrEL control formulation. By using the experimental data from the CrEL control group as an anchor for dissociated drug, the model simulates the contribution of DcNP association to the observed increase in plasma concentrations and is reported in FIGS. 8A and 8B.

### Analysis of MBPK Structure and Assumptions

Individual distribution, metabolism and elimination of G and T are well characterized. G undergoes rapid and complete deamination to inactive metabolite (dFdU) by ubiquitous CDA. T is metabolized in the liver primarily by CYP3A4 and CYP2C8 with subsequent excretion of metabolites into the bile. In this model, the k_{0,1} term represents the aggregate elimination processes of G and T through their independent described pathways. A peripheral compartment for both G and T was added and distribution was parameterized with the k_{1,2} and k_{2,1} terms. With these considerations, a parallel compartment was added to represent DcNP associated drug and assumed the following: [1] at the moment of injection, G and T are both completely associated to DcNPs, but are released at different rates; [2] apart from release, there is no other mechanism of clearance for drug bound to the DcNP; [3] when either G or T has been released from DcNPs their pharmacokinetic behavior will be the same as that of the CrEL control and [4] the amount of drug released from particles in the peripheral compartment is negligible. With these assumptions in place, the k_{1,3} term was set to link the two sub-models and represent the release mechanism of drug from DcNP into the central compartment. The DcNP associated species of G and T was then parameterized with the k_{4,3} and k_{3,4} to account for distribution. By fitting the additive sum of compartments 1 and 3 to the observed GT concentrations, the fraction of drug that is associated or dissociated in plasma and the relevant pharmacokinetic parameters was estimated.

Model Simulations and Verification with Experimental Data

For G, the estimated volume of central compartment decreased 4.6 times when administered as DcNP compared to administration in CrEL form(24 mL vs 5.2 mL) which likely reflects a reduced distribution of DcNP-G into tissues. For T, the estimated volume of distribution was slightly less than the physiological plasma volume of a mouse (˜0.8 mL) in both DcNP and CrEL groups. It is likely that T association to DcNPs or CrEL micelles limits the distribution of T from the plasma. Thus, the volume parameter was fixed at 0.8 mL to retain physiological context. It is important to note that the dissociated T parameters were derived from an injection of the CrEL control and not completely soluble drug (due to solubility limitations) and the CrEL micelles may limit distribution of T from plasma. The estimated release parameter of G (k_{1,3}) was 9.5-fold lower than T (0.2 hour⁻¹ vs 1.9 hour⁻¹, Table 4) and corresponds to the relative 11.3-fold difference in mean residence times of G and T after DcNP administration (11.3 hours vs 1.0 hour, Table 3). Based on the parameters generated in this model (Table 4), a simulation was performed to predict the plasma concentration time course kinetics of dissociated and associated G and T (FIGS. 8A and 8B). The ratio of dissociated over total G AUCs as simulated by the model was 1.5%, which agrees with our maximum fraction dissociated in plasma estimate of 1.6% from Eq. 1. For T, the model simulated ratio of dissociated over associated T AUCs is 24.7% and in close agreement with our boundary estimate using Eq. 1 (26.6%). It is interesting to note that the in vivo association of G is greater than T (˜98% vs ˜73%), but the opposite is true for in vitro association (9% versus 95%). There is likely an unknown in vivo mechanism that enables the substantial association of G to DcNPs, however further investigation is required. Despite these unknowns, the current results clearly show that DcNPs retain hydrophilic G and hydrophobic T together in plasma (up to 8 hours) and may enable the co-delivery of GT to target cancer cells.

TABLE 3
Effect of DcNP formulation on pharmacokinetic parameters of gemcitabine and
paclitaxel administered together in GT DcNP or control suspension.
Pharmacokinetic parameter estimates were derived from the data shown in FIG. 4.
^{a}Non compartmental analysis was used to estimate area under the curve (AUC) from 0 to
∞ of gemcitabine and paclitaxel in DcNP and CrEL control.
^{b}Apparent half-life was estimated from the slope of the last 3 time points collected in each
condition.
^{c}Concentration at time 0 was back extrapolated from the first time point in each condition.
^{d}Dose/AUC is shown for the relative apparent clearances of gemcitabine and paclitaxel.
^{e}Vₛₛ was calculated using Dose*AUMC/AUC² extrapolated to infinity
^{f}Mean residence time (MRT) was calculated by dividing the area under the moment curve
(AUMC) by the AUC from 0 to infinity.

| | | Gemcitabine | | | Paclitaxel | | |
|---|---|---|---|---|---|---|---|
| Parameter | Units | Control | DcNP | Ratio | Control | DcNP | Ratio |
| AUC_{0 to ∞}^{a} | μg | 920.8 | 56218.6 | 61.0 | 156.5 | 588.8 | 3.8 |
| | min/ml | | | | | | |
| T_{1/2,app}^{b} | hr | 1.6 | 13.7 | 8.6 | 1.8 | 2.0 | 1.1 |
| C₀^{c} | μg/mL | 165.1 | 181.4 | 1.1 | 17.7 | 13.9 | 0.8 |
| Dose/AUC^{d} | mL/hr | 65.2 | 1.1 | 0.02 | 38.3 | 10.2 | 0.3 |
| Vss^{e} | mL | 16.6 | 12.1 | 0.7 | 35.6 | 10.0 | 0.3 |
| MRT^{f} | hr | 0.25 | 11.3 | 45.2 | 1.0 | 1.0 | 1.0 |

TABLE 4
Model derived pharmacokinetic parameters for gemcitabine and paclitaxel
when administered as a single IV dose in GT DcNP dosage form.

| DcNP Dissociated Parameters | Gemcitabine | Paclitaxel |
|---|---|---|
| V (mL) | 24 ± 12 | 0.8^{a} |
| K_{0,1} (1/hour) | 3.9 ± 0.9 | 7.0 ± 2.0 |
| K_{1,2} (1/hour) | 0.5 ± 0.2 | 0.6 ± 0.1 |
| K_{2,1} (1/hour) | 0.2 ± 0.1 | 4.4 ± 1.0 |
| DcNP Associated Parameters | Gemcitabine | Paclitaxel |
| V_{DcNP} (mL) | 5.2 ± 0.6 | 0.8^{a} |
| K_{1,3} (1/hour) | 0.2 ± 0.02 | 1.9 ± 0.1 |
| K_{3,4} (1/hour) | 0.6 ± 0.1 | 0.7 ± 0.2 |
| K_{4,3} (1/hour) | 1.8 ± 0.5 | 1.6 ± 0.5 |

^{a}Volume was fixed to plasma volume assuming a blood volume of~2 mL for 8 to 9-week-old mice and a hematocrit of 40%

### Effect of DcNP Formulation on Gemcitabine and Paclitaxel Tissue Distribution

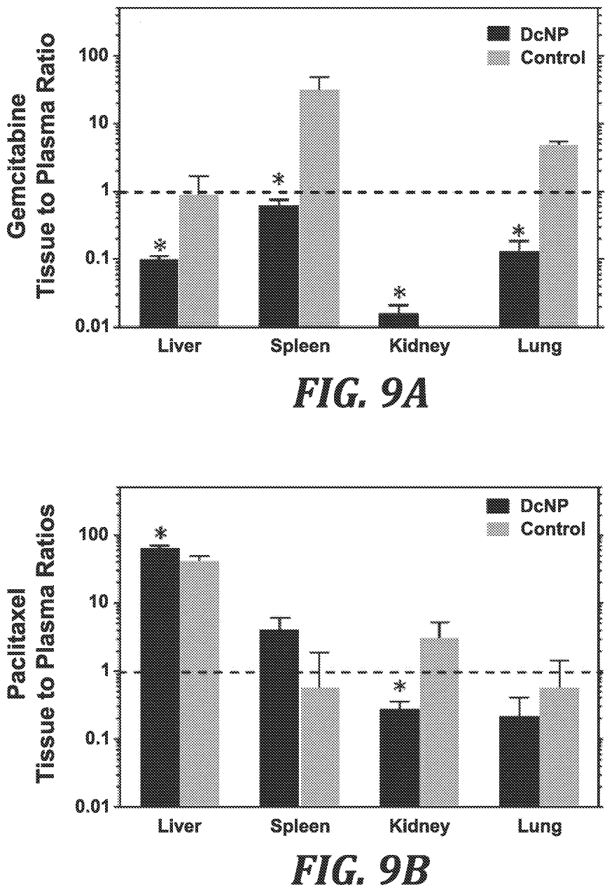

The effect of DcNP on preferential GT tissue distribution in mice was determined. Non-specific, off-target accumulation of drugs in healthy tissues can limit the therapeutic potential of GT DcNPs. In addition, accumulation of cytotoxic drug in healthy tissues can pose a safety concern. Therefore, tissue-to-plasma drug concentration ratios were compared at 3 hours after IV administration in mice dosed with GT in DcNP or the CrEL control dosage form. The 3-hour time point was selected to ensure that both drugs are detectable in both plasma and tissues for animals treated with DcNP or CrEL control. As shown in FIGS. 9A and 9B, DcNPs retain G in the plasma relative to CrEL control 3 hours post-injection in all tissues tested (p<0.05, Student's T-Test). For T, lung and kidney tissue-to-plasma ratios were reduced in DcNP vs CrEL control; while liver and spleen ratios increased. These data indicate that G in DcNPs does not accumulate in off-target organs such as the liver and spleen and, instead, GT in DcNP are better retained in blood and plasma. The effect of DcNPs on T distribution is less dramatic but there does not appear to be a substantial trend towards healthy organ accumulation (FIGS. 9A and 9B). Taken together, these data suggest that G bound to DcNPs does not accumulate in any of the sampled tissues and likely provides drug associated to DcNP a greater opportunity to reach target tumor cells by remaining in the systemic circulation. It is possible that T binding to serum protein, as well as stripping of T from DcNPs, may contribute to the differential tissue distribution of G and T in healthy mice. However, this difference, particularly the T increase in liver and spleen, is minimal. The large reduction of G in the four off-target tissues are significant compared to that of CrEL control formulation and may result in less off-target toxicity.

### Effect of DcNP Formulation on Gemcitabine and Paclitaxel Localization in Healthy Versus Tumor Bearing Lung Tissue

To determine whether DcNPs enhance GT distribution into target tissues, namely cancer nodules, the differential localization of GT in DcNP or CrEL form in healthy versus cancer nodule bearing mice was investigated. Since lungs are a common metastatic site for breast cancer disease progression, a model for metastasis that forms cancer nodules in the lungs was used. Cancer nodules were established in female BALB/c mice via intravenous inoculation of syngeneic breast cancer cells (4T1). This process has been shown to consistently produce detectable and multiple 4T1 cancer nodules in lungs within 14 days. Once nodules were established and confirmed with IVIS imaging, mice were administered with GT in either DcNP or conventionally solubilized form at equivalent doses (50/5 mg/kg, GT) and euthanized at pre-determined time intervals. Nodule-free and nodule-bearing lungs were harvested, and lung tissue drug concentrations were compared in these two groups.

In mice bearing 4T1 cancer nodules, a higher concentration of G (p<0.01) and T (p<0.12) is observed in lung tissue 1 hour after CrEL administration compared to healthy tissue. Increases of lung concentration reflect increases of plasma concentration of G (p=0.14) and T (p<0.01). Comparison of lung-to-plasma ratios 1 hour after CrEL administration shows that G (p=0.24) and T (p=0.4) distribution are not significantly different between healthy and cancer nodule burdened animals. For cancer nodule burdened mice given DcNPs, no statistical significance was observed in lung, plasma, or lung-to-plasma ratios. At later time points (24 hour), free drug rapidly clears from the systemic circulation and only the DcNP group has detectable levels of drug in plasma and lung. The lung-to-plasma ratios of G in cancer nodule bearing mice at 24 hours are 5 times greater than in healthy with a p-value of 0.02. Although a lung-to-plasma ratio of T cannot be determined at 24 hours due to rapid plasma clearance, the concentration of T is 7×greater in cancer nodule bearing mice versus healthy mice (p=0.008). These data are summarized in Table 5. The results from this experiment show that when administered to healthy mice, DcNPs can sustain drug levels in the lung (a major site of metastasis) for a longer time than the CrEL control formulation. When mice have cancer nodules present, G and T levels in the lung are increased relative to healthy mice. These increases in lung concentrations are disproportionately larger than the elevated concentrations in plasma, suggesting that the particles preferentially target cancer burdened tissue. Furthermore, when the GT concentration ratios in lungs were compared, an 8.8 to 1 ratio is observed, similar to the original drug ratio in formulation. This further supports the idea that particles may selectively deposit and retain in cancer burdened tissue.

### Discussion

A key challenge in the treatment of breast cancer is metastasis and poor tolerability of highly potent, chemotherapeutic drug combinations. Off-target drug distribution and asynchronous concentrations of drug combinations in target tissues (tumors) likely contribute to the high dose requirements for metastatic control and dose-limiting toxicities. To coordinate the anticancer effects of two chemotherapeutic agents, we have successfully co-formulated chemically distinct gemcitabine (G) and paclitaxel (T) in a drug combination nanoparticle (DcNP). When intravenously given to mice laden with metastatic 4T1 breast cancer nodules in the lung, the GT DcNP demonstrated improved tissue selectivity and long-acting exposure of both drugs in metastatic cancer bearing tissues (Table 5).

TABLE 5
Effect of 4T1 tumors on gemcitabine and paclitaxel localization in tumor burdened
lung tissues after dosing with GT DcNP or CrEL control formulation
Data in the table are presented as the geometric mean of three biological replicates ±
standard deviation. No drug was detected with the CrEL control 24 hours after drug
administration.
*denotes p < 0.05.
NA denotes a ratio that is not calculable
BDL denotes no detectable drug in the sample

| Gemcitabine | | | Paclitaxel | | |
|---|---|---|---|---|---|
| Healthy | 4T1 | p-value | Healthy | 4T1 | p-value |

1 hour after control

| | | | | | | |
|---|---|---|---|---|---|---|
| Lung _{(μg/g)} | 0.9 ± 0.4 | 6.0 ± 0.8 | <0.01* | 1.0 ± 0.3 | 1.7 ± 0.5 | 0.12 |
| Plasma _{(μg/mL)} | 0.9 ± 0.2 | 2.8 ± 1.4 | 0.14 | 0.3 ± 0.05 | 0.7 ± 0.1 | 0.01* |
| Lung/Plasma Ratio | 0.9 ± 0.3 | 2.2 ± 1.4 | 0.24 | 3.3 ± 1.2 | 2.5 ± 0.8 | 0.40 |

1 hour after DcNP

| | | | | | | |
|---|---|---|---|---|---|---|
| Lung _{(μg/g)} | 7.5 ± 1.2 | 12.5 ± 4.9 | 0.21 | 0.6 ± 0.4 | 0.9 ± 0.5 | 0.46 |
| Plasma _{(μg/mL)} | 52.2 ± 64 | 72.6 ± 60.0 | 0.71 | 1.4 ± 0.9 | 2.2 ± 1.1 | 0.39 |
| Lung/Plasma Ratio | 0.1 ± 0.2 | 0.2 ± 0.1 | 0.85 | 0.4 ± 0.01 | 0.4 ± 0.2 | 0.92 |

24 hours after DcNP

| | | | | | | |
|---|---|---|---|---|---|---|
| Lung _{(μg/g)} | 0.3 ± 0.1 | 3.5 ± 1.0 | 0.01* | 0.06 ± 0.01 | 0.4 ± 0.1 | 0.03* |
| Plasma _{(μg/mL)} | 12.7 ± 1.5 | 34.2 ± 0.6 | <.01* | BDL | BDL | NA |
| Lung/Plasma Ratio | 0.02 ± .01 | 0.10 ± .03 | 0.04* | NA | NA | NA |

Interestingly, the single nanoparticle composed of the unlikely partners—water soluble G and water insoluble T—not only demonstrated long acting tissue selectivity for breast cancer nodules in the lung, but also minimal distribution into healthy organs. The tissue-to-plasma ratios of GT DcNPs, which also produced long acting plasma circulation, did not significantly differ from the control GT administration in mice 3 hours after IV injection (FIGS. 9A and 9B). An unexpected finding is that both G and T remain well associated to DcNPs for the duration of the time course study, which may be related to target tissue localization and the long-acting pharmacokinetics of GT enabled by the DcNP platform. In vitro association efficiency suggests that paclitaxel has a greater affinity for DcNPs than does gemcitabine. However, when given in DcNP form, gemcitabine has a greater enhancement in plasma exposure than paclitaxel compared to their respective controls. Pharmacokinetic modeling and simulation was used as a novel tool to distinguish the in vivo associated and dissociated fractions of gemcitabine. This approach may be used to estimate the associated and dissociated fractions of drug over time for other nanoparticle drug delivery systems where isolation of in vivo associated and dissociated drug is experimentally challenging. Although this work focuses specifically on the use of combination G and T, DcNPs represent a potential approach to the synchronized delivery of other combination regimens used in breast cancer treatment such as targeted therapy or hormone therapy.

Combination drug nanoparticles have been previously reported as potential therapies for cancer. However, it remains a challenge to co-formulate chemically dissimilar drugs such as G and T (water soluble and insoluble drugs). It is believed that there are only a few published reports that achieve the co-formulation of GT to target breast cancer and each study notes an improved effect of combination particles versus individual GT which highlights the potential for combination particles. Water soluble G and water insoluble T are brought together by approaches such as chemical conjugation of both drugs to polymers or encapsulation in calcium phosphate nanoparticles with a lipid bilayer coating. However, chemical conjugation produces a new chemical entity that requires a long journey of regulatory approval and filing as a new drug. Calcium phosphate precipitation requires multiple filtration steps to remove organic solvents such as THF or chloroform.

In contrast, the distinction of the DcNP process is that no chemical conjugation is required to produce substantial in vivo association of both gemcitabine and paclitaxel. The DcNP process does not require filtration of unassociated drug or co-solvents as described in other reports. Even with a limited AE % of 9% for gemcitabine, a 50-fold increase in gemcitabine plasma exposure is observed when compared to the CrEL control. Further analysis based on a combination of analysis of metabolite kinetics and MBPK modeling suggests that water soluble gemcitabine is highly associated with DcNP in vivo. Paclitaxel was found to be highly associated both in vitro and in vivo, although the lower dose (5 mg/kg) limits its duration in plasma. The stable circulation of GT well associated to DcNPs in plasma demonstrates the ability of one carrier to load two anticancer drugs while targeting cancer cells (FIGS. 6A and 6B).

Clinical studies have shown that prolonged infusion rates of G (10 mg/m²/min) confer a survival advantage over standard 30-minute infusions. Deoxycytidine kinase (dCK), which converts G to its active triphosphate form, has been shown to be rapidly saturated after G infusion. As a result, a large fraction of the total dose of G is lost to metabolism by CDA before activation by dCK. Increasing the infusion time of G can allow more drug to be converted to active form and produce a greater pharmacologic effect.

In the present Example, a single dose of GT in DcNPs increased the apparent plasma half-life of G from 1.6 hours to 13.72 hours in mice (FIGS. 6A and 6B). No infusion is necessary in this case with GT DcNP administration. Although total drug concentration in plasma does not directly reflect the free fraction of G available for phosphorylation, the persistent circulation of parent drug can increase the opportunity for drug to reach target cells for phosphorylation instead of inactivation as seen with other long-acting nucleoside analogs. Thus, extending the plasma circulation of parent G may act similarly as a prolonged infusion and may produce a greater pharmacologic effect. Regarding T, clinical studies have not established a relationship between infusion rate and pharmacologic effect. Instead, conventional Taxol is infused over 3 hours to mitigate the toxicity of Cremophor EL, a solubilizing excipient. Minor and major hypersensitivity reactions have been linked to rapid infusion of Cremophor El. When this excipient is not present, such as in albumin bound paclitaxel formulations (Abraxane), infusions can be administered in as little as 30 minutes without prophylactic medications for hypersensitivity reactions. In the present Example, the use of biocompatible lipid excipients with proven human safety in other dosage formulations enables T to stay suspended in nanoparticle form. T in DcNP can be administered in a single dose (with G) without the need for Cremophor EL.

The pharmacokinetic profiles of nanoparticle delivery systems are often described using total drug concentrations instead of unbound drug concentrations. This is partly due to the complexity of separating bound and unbound fractions of drug from biological matrices. Total drug concentrations can provide an adequate description of particle circulation but may confound the prediction of pharmacologic effect. In this Example, the fraction of drug that is associated to nanoparticles in vivo was estimated. GT association to DcNPs was first estimated by in vitro dialysis under sink conditions (G: 9%, T=95%). However, this estimate did not correspond with the in vivo results and may be due to the lack of blood components in the dialysis experiment, which can affect drug dissociation.

In the biologic milieu and limited blood volume, the in vivo data indicates an association of G to DcNPs much greater than the 9% found in vitro. These differences were reconciled by using a non-compartmental approach to estimate the maximum time-averaged fraction of dissociated drug that can be present in vivo (Eq. 1, f_{diss. max}). This approach was applied to an estimate for the fraction of drug available for metabolism when administered as a DcNP (Eq. 2, f_{diss.G→dFdU}) For G, the estimate for f_{diss.G→dFdU} was greater than f_{diss. max} suggesting that metabolism for DcNP-G can occur in tissues that are not part of the central compartment (such as lean tissue). It is possible that DcNPs distribute into peripheral tissue where local dissociation and metabolism of G can occur. Since this loss of parent drug does not re-enter the systemic circulation, Eq. 1 may underpredict the dissociated fraction. Alternatively, Eq. 2 captures the transit of dFdU from peripheral tissue back into the central compartment and account for peripheral metabolism but both f_{diss. max} and f_{diss.G→dFdU} indicate that G is highly associated to DcNPs. Regarding T association to DcNPs, the clearance pathway for dissociated T is in the liver, which resides in the central compartment. Under these conditions, f_{diss. max} can provide an estimate of the dissociated fraction without needing to account for loss of parent drug in peripheral tissues; it shows that T is also highly associated to DcNPs in vivo. Taken together, both estimates show that G and T mostly circulate in vivo as associated forms.

As an extension to those non-compartmental estimates, a mechanism-based pharmacokinetic model (MBPK) was adapted to derive a dynamic simulation of both G and T association to DcNPs in plasma. Results from this MBPK model showed that both hydrophilic G and hydrophobic T are well associated in vivo and correlate closely to the non-compartmental estimates. These early results demonstrate a novel application of pharmacokinetic modeling to understand the species of GT that circulate in vivo.

GT DcNPs did not appear to accumulate in healthy organs. Depending on the composition and size of nanoparticle formulations, the liver and spleen can sequester as much as 99% of other types of nanoparticles. This is mainly due to the fenestrations in liver and spleen microvasculature and direct interactions of traditional nanoparticles with endocytic cells. Although the removal of particulates constitutes an essential component of the immune system, the premature clearance of particles prior to their interaction with target cells poses a barrier to effective nanoparticle delivery. Lipid-based particles such as liposomes are often associated with liver and spleen uptake. For example, when large (378 nm) and small liposomes (113 nm) were intravenously administered in mice, 93% of large liposomes and 67% of small liposomes were recovered in the liver and spleen after 4 hours. The hepatic uptake of particles is also observed with non-lipid nanoparticle delivery systems. For example, intravenously administered albumin-bound paclitaxel (Abraxane, 30 mg/kg) in mice found that after 3 hours, liver concentrations of paclitaxel were 100-fold greater than plasma concentrations. In the same study, an F127 stabilized nanocrystal form of paclitaxel was intravenously injected (30 mg/kg) in mice and after 3 hours, liver concentrations were 50-fold greater than plasma. Compared to these liposome and nanoparticle drug delivery systems, the GT in DcNP form have remarkably different biodistribution characteristics.

GT DcNPs are a combination particle that contains multiple active drugs to overcome drug resistance unlike single drug particles. Both water soluble G and water insoluble T are stabilized together by two lipid excipients, without the need for a membrane structure such as liposomes. Electron microscopic analysis of GT DcNP product reveals that they contain neither membrane structures nor spherical enclosures typically observed with liposomes. After IV injection, GT DcNPs do not appear to distribute into or accumulate in the liver or spleen. For G administered as DcNPs, the tissue-to-plasma ratios show that DcNPs significantly limit tissue distribution compared to the control formulation (<1/10^{th} across all tissues) (FIG. 9A). This effect on G disposition is particularly promising as hepatic distribution of free drug is associated with hepatic abnormalities and dysfunction. For T, there is no clear trend on tissue distribution of drug when administered as a DcNP compared to the conventionally solubilized form. T is known to interact readily with albumin, while G does not, and this drug specific property may affect T disposition. Regardless of how T can dissociate from combination nanoparticles, the overall accumulation of both G and T in off-target organs is minimal compared to previous reports and may lead to improved safety.

In metastatic breast cancer, solid tumors and metastatic nodules induce major changes to their surrounding microenvironment. These changes can limit the effectiveness of nanoparticle delivery systems by reducing penetration into solid tumors. Various approaches have been investigated to overcome these limitations such as active targeting and tumor priming with limited clinical success. In our study, we found that intravenously administered DcNPs can produce greater concentrations in tumor burdened pulmonary tissue compared to healthy pulmonary tissue. This enhancement of drug accumulation in tumor-bearing lungs is likely due to increased distribution of DcNP from plasma to peripheral tumor fenestrations within tumor foci. The small size (60-70 nm) and prolonged circulation (48 hours) in plasma of DcNPs may allow particles to penetrate the fenestrations of tumor foci, which typically range from 0.3 to 4.7 microns in size. Other nanoparticle systems such as liposomes or polymeric particles have been reported to leverage the leaky neovasculature around tumors to enhance drug permeation and retention (commonly referred to as the EPR effect). In these scenarios, new blood vessels formed to support rapid tumor growth are leaky due to poorly developed endothelial cells lining the vessels. This allow for the passive distribution, diffusion, or penetration of nano-sized particles into solid tumors. In the present Example, metastatic 4T1 cancer cells present as lung cancer nodules in already highly perfused capillary beds. Under these conditions, it is not clear whether neovasculature has formed or the role of the EPR effect on the observed tumor tissue targeting by GT DcNPs. Nevertheless, the data suggest the selective deposition of GT DcNPs in tumor burdened tissue. When the concentration ratios of G and T in tumor burdened lungs, a ratio similar to the administered dose (8.8:1 versus 10:1, G/T). This suggests that intact DcNPs are depositing in tumor bearing lungs without having a large fraction of the dose sequestered in the reticuloendothelial system, reflected in the liver and spleen. The observation that untargeted DcNPs can have a tumor specific deposition in pulmonary tissue is a promising feature of DcNPs. While one could seek to improve the dispositional advantage of GT DcNPs with the use of active targeting ligands (such as those targeted to EGFR, integrin, or other MBC markers), such studies are beyond the scope of this report but are under consideration for future studies.

The therapeutic effects of this GT DcNP composition on 4T1 breast cancer metastasis to lungs in mice was evaluated. A single GT (20/2 mg/kg) dose in DcNP form nearly eliminated breast cancer colonization in the lungs, while this effect was not achievable by a CrEL drug combination at a 5-fold higher dose (i.e., 100/10 mg/kg GT). Dose-response curves of cancer nodule inhibition and systemic toxicity through body weight loss demonstrated a therapeutic index of about 15.8. These results may be related to the preferential distribution and long acting pharmacokinetic properties contributed by stable association of GT to DcNPs in vivo.

Thus, stable drug combination nanoparticles composed of water-soluble gemcitabine and water insoluble paclitaxel was developed. GT DcNP stabilization is enabled by lipid excipient composition and a novel but simple process that does not require complex free drug removal. By doing so, this highly potent combination of chemotherapy has been transformed from a short-acting regimen to a long-acting regimen. The development and application of a mechanism based pharmacokinetic model elucidates the time course of associated and dissociated fractions of GT in vivo. This validated model indicates that GT remains associated to DcNP in vivo and displays an enhanced distribution toward cancer burdened tissue over healthy tissue, which may improve the therapeutic effect of this combination. The DcNP platform is able to incorporate multiple drugs and allow water soluble and insoluble chemotherapeutic agents to form a single nano-dosage form. Thus, it may be used for other cancer drug combinations, either in clinical use or in development, with higher potencies. In addition, it may be possible to incorporate targeting ligands in the DcNP to provide additional cancer cell selectivity and preferential distribution of the chosen drug combinations. The long-acting and cancer tissue selective drug combination kinetics provided by this DcNP platform technology may lead to a meaningful impact on the development of targeted, combination treatment of metastatic breast cancer.

### Example 3 Design and Characterization of a Novel Venetoclax-Zanubrutinib Combination in a Long-Acting Injectable Nanoformulation

Venetoclax and the second-generation anti-BTK drug, zanubrutinib, were used as candidates for our drug platform. Venetoclax and zanubrutinib are administered orally, a route that patients usually prefer over parenteral routes, though the oral route can limit a drug's efficacy against disease. Gastrointestinal (GI) absorption of the drugs can be restricted due to metabolic enzymes in the gut and liver prior, leading to a low drug bioavailability, sub-therapeutic drug plasma and intracellular concentrations, and the subsequent promotion of drug resistance due to insufficient drug concentrations at the cancer site. In addition, orally delivered drug requires daily dosing, which can be cumbersome for the patient and leads to gastrointestinal injury due to constant high drug levels in the GI tract. To overcome these limitations, a system in which lower amounts of drug could be delivered over an extended period of time would greatly improve both toxicity against the disease as well as patient tolerance of drug. A drug combination nanoparticle (DcNP) platform can effectively accommodate both venetoclax and zanubrutinib, creating a drug delivery system that has reduced drug load (due to synergistic drug interactions) and extended systemic exposure (due to lymphatic retention of the DcNP's).

### Methods and Materials

### Reagents

1,2-Distearoyl-sn-glycero-3-phosphocholine (DSPC) and N-(carbonylmethoxypolyethyleneglycol-2000)-1,2-distearoyl-sn-glycero-3-phosphoethanolamine, sodium salt (DSPE-mPEG₂₀₀₀) were purchased from Corden Pharma (Liestal, Switzerland). Venetoclax (ABT-199) and zanubrutinib (BGB-3111) were purchased from MedChemExpress (Monmouth Junction, USA). All other chemicals and solutions are from Sigma (St. Louis, USA) unless otherwise noted.

### Production of Drug-Combination Nanoparticles

85.4 mg DSPC, 33.6 mg DSPE-mPEG₂₀₀₀, 9.6 mg ABT-199, and 9.6 mg BGB-3111 were dissolved in 4 mL pre-warmed 70° C. tert-butyl alcohol. The solution was thoroughly mixed, lyophilized over 24 hrs, and reconstituted in 0.9% NaCl, 20 mM NaHCO₃ buffer with trace TWEEN® 20. The resulting drug-combination, stabilized by lipid excipients and referred to as drug combination nanoparticle or DcNP's, were then filtered through an Acrodisc® CR syringe filter (0.2 μm PTFE Membrane, HPLC certified) and diluted with buffer solution to the desired concentration. Particle size was quantified using a NICOMP 380 ZLS (Zeta Potential/Particle Sizer). The extent of ABT-199 and BGB-3111 association with the DcNP particles in solution was analyzed via dialysis (6,000-8,000 molecular weight cutoff) under sink conditions; sink condition was achieved by dialyzing 200 μl of DcNP suspension against 200 mL buffer solution (1,000-fold volume change) for 4 hrs. The drug association efficiency (AE %) was determined by comparing the pre- and post-dialysis drug concentration ratios of each drug. Drug concentration was determined by the extraction drug in suspension and analyzed based on a LC-MS/MS assay as described below.

### Drug Extraction and LC-MS/MS Analysis of Drug Concentrations

To quantify drug concentrations (both DcNP-bound and free drug), an extraction protocol was established to quantify venetoclax and zanubrutinib. In short, drug was solubilized by diluting the sample into ethyl acetate, liberating it from either the DcNP lipid matrix, mouse plasma, or both. Following centrifugation to remove organic material, the samples were dried with nitrogen gas, reconstituted in acetonitrile, and loaded onto an LC-MS/MS. Drug concentrations were quantified by comparing sample data with standard curves prepared from untreated mouse plasma spiked with known amounts of drug.

Separations were carried out on a Synergi column (100×2.0 mm) (Phenomenex, Torrance, USA). A C₈ guard column (4.0×2.0 mm) was used (Phenomenex, USA). The separations were done under ambient temperature, and the flow rate was set to 0.55 ml/min. The mobile phase for the separations consisted of buffers A (water with 20 mM ammonium acetate) and B (acetonitrile). The single mobile phase ran for five minutes, and it consisted of 25% buffer A and 75% buffer B.

### Assessing Drug Potency Against Cancer Cell Growth

K-562 cells (human leukemia) were purchased from ATCC (Manassas, USA). Additional human leukemic cell lines, MOLT-4 and HL-60, were a generous gift from Carrie Cummings at Fred Hutchinson Cancer Research Center (Seattle, USA). All cells were cultured in Gibco RPMI medium 1640 with Gibco 1% 100×Antibiotic-Antimycotic (Thermo Fisher Scientific, Waltham, USA) and 10% fetal bovine serum. Cells were selected for their different protein expression levels of Bruton's Tyrosine Kinase (BTK) and B Cell Lymphoma 2 (Bcl-2); HL-60 cells express both BTK and Bcl-2, while K-562 and MOLT-4 cells only express BTK and Bcl-2, respectively.

Each cell line was seeded separately into Costar® Black 96-well Assay Plates (Corning USA). Within 1 hr, varying concentrations of individual free drug (ABT-199 or BGB-3111), a combination of both free drugs (w/w 1:1), or a combination of drugs within DcNP's were added to the cells. Following a 5-day incubation, growth of treated cells was compared to untreated cells, quantified using an AlamarBlue Cell Viability Assay (Thermo Fisher Scientific, Waltham, USA) with a PerkinElmer 1420 Multilabel Counter plate reader. Prism graphing software (GraphPad) was used to analyze the absorbance data and to assess relative cell growth.

### Leukemic Cell Uptake and Retention of Free Versus DcNP-Associated Drug

HL-60 cells were cultured, counted, and aliquoted into multiple 1.5 mL Eppendorf tubes. A free drug solution of ABT-199 and BGB-3111 (1:1 w/w) was added to half of the tubes, while a DcNP solution of identical drug concentrations was added to the second half of tubes. The cells in the tubes were then allowed to incubate normally. At preselected time points, one incubation tube from each group was removed from the incubator, and the cells inside were washed twice with media to remove external drug and DcNP's. Cells were then lysed with acetonitrile, and drug concentrations were quantified according to the aforementioned extraction and LC-MS/MS protocol.

### Pharmacokinetics of DcNP's Versus Free Drug

All animal procedures complied with and were approved by the University of Washington Institutional Animal Care and Use Committee. Female BALB/C mice were used, originally obtained from Charles River Laboratories (Wilmington, Mass.). Mice were kept under pathogen-free conditions, exposed to a 12 h light/dark cycle, and received food ad libitum. Three groups of three mice each were administered ABT-199 and BGB-3111: (1) the first group received a 180 μL intravenous injection of 600 μg ABT-199 and 600 μg BGB-3111 in 0.9% NaCl, 20 mM NaHCO₃ buffer with trace DMSO and Cremophor EL as solubilizing agents, (2) the second group received an intravenous injection of ABT-199 and BGB-3111 DcNP's in equivalent volume and drug molar concentration as the first group, and (3) the third group received a subcutaneous injection of ABT-199 and BGB-3111 DcNP's in the inner right leg. Plasma was collected at select time points, then extracted and analyzed by HPLC-MS/MS using the previously established protocol to determine plasma concentrations of drug over time.

### DcNP's Particle Size Over Time

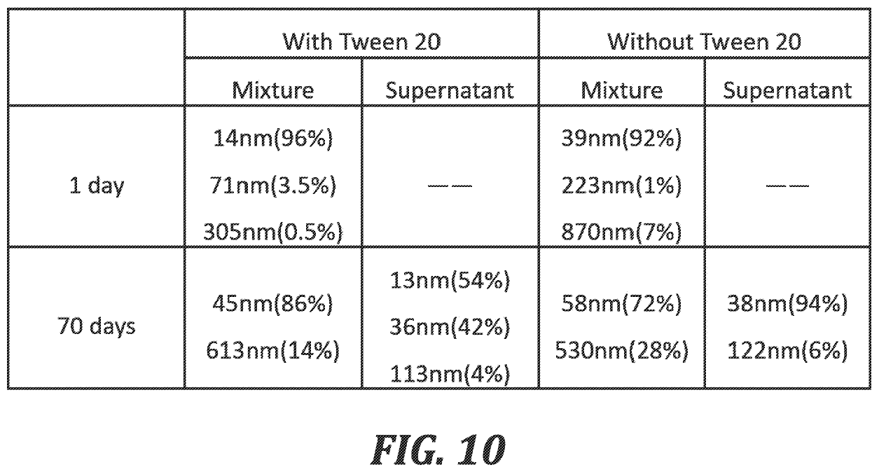

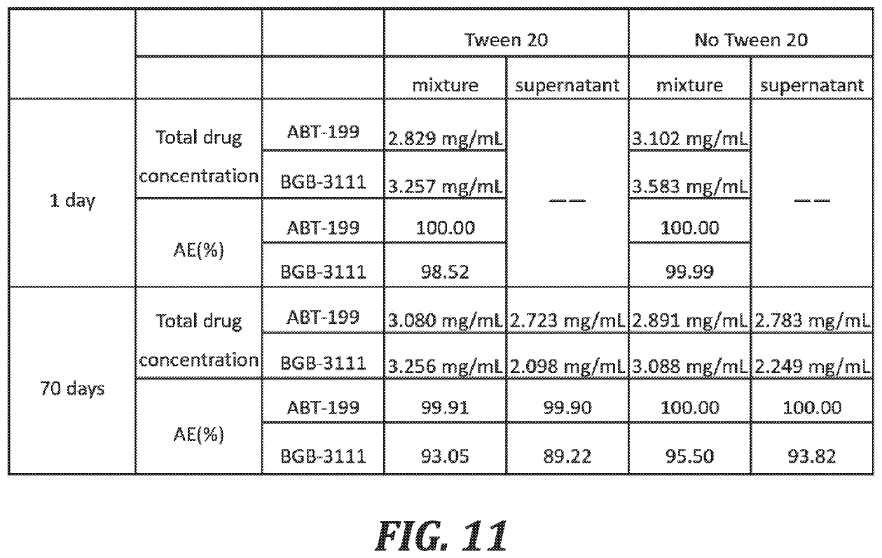

Following initial rehydration and without any sonication, particle size of the DcNP's with and without TWEEN20 was measured using the Zetasizer (FIG. 10). In the presence of TWEEN20, particle size (diameter) was found to be primarily 14 nm (96%) with less than 4% of particles measuring 71 nm. Without Tween20, DcNP size was primarily 39 nm in diameter (92%) with less than 8% of particles measuring 870 nm in diameter (FIG. 10). Particle size was determined via dynamic light scattering, and association efficiency (FIG. 11) is a normalized mass/mass ratio calculated by comparing amount of drug remaining associated with DcNP's compared to the overall amount of drug used to create the particles.

After rehydration, particles were left untouched for 70 days. DcNP's precipitate naturally over time; both the particle size of DcNP's in the “supernatant” of the mixture prior to mixing as well as the DcNP's in solution following mixing were again measured using the Zetasizer (FIG. 11). After gently mixing the precipitate back into solution, average particle size of the TWEEN20-containing mixture increased to 45 nm (86% of DcNP's) with the remaining 14% of particles measuring around 613 nm. Without TWEEN20, particle size also increased over the 70 day period to 58 nm (72% of DcNP's) with the other particles in solution measuring 530 nm (28% of DcNP's) (FIG. 10).

### Association Efficiency of Venetoclax and Zanubrutinib Over Time

After both initial rehydration and a 70-day rest period, the association efficiency (AE %) of venetoclax and zanubrutinib with the DcNP lipid platform was examined.

With TWEEN20, AE % of venetoclax was 100% on day 1, which decreased to 99.91% on day 70. Without TWEEN20, AE % of venetoclax was 100% on day 1, which remained at 100% on day 70 (FIG. 11).

With TWEEN20, AE % of zanubrutinib was 98.52% on day 1, which decreased to 93.05% on day 70. Without TWEEN20, AE % of zanubrutinib was 99.99% on day 1, which deceased to 95.50% on day 70 (FIG. 11).

### In Vitro Inhibition of Leukemic Cell Growth by Free and DcNP-Bound Venetoclax and Zanubrutinib

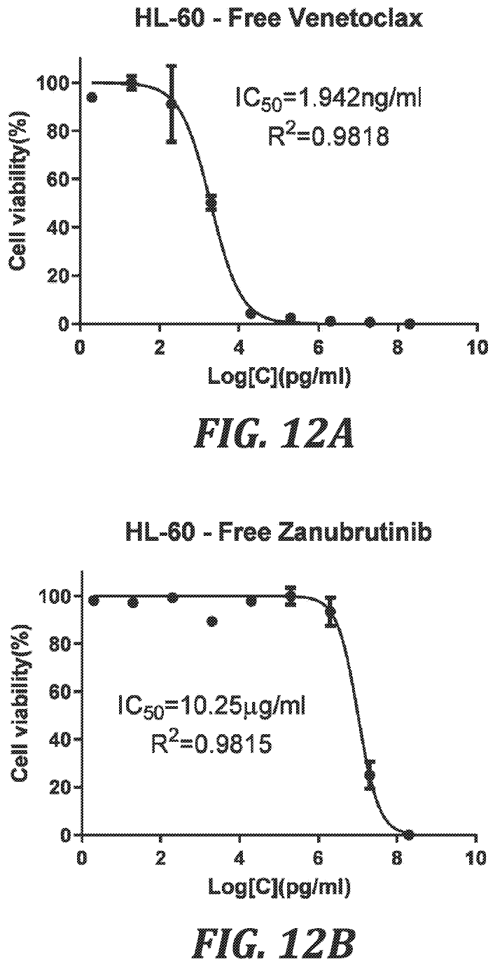

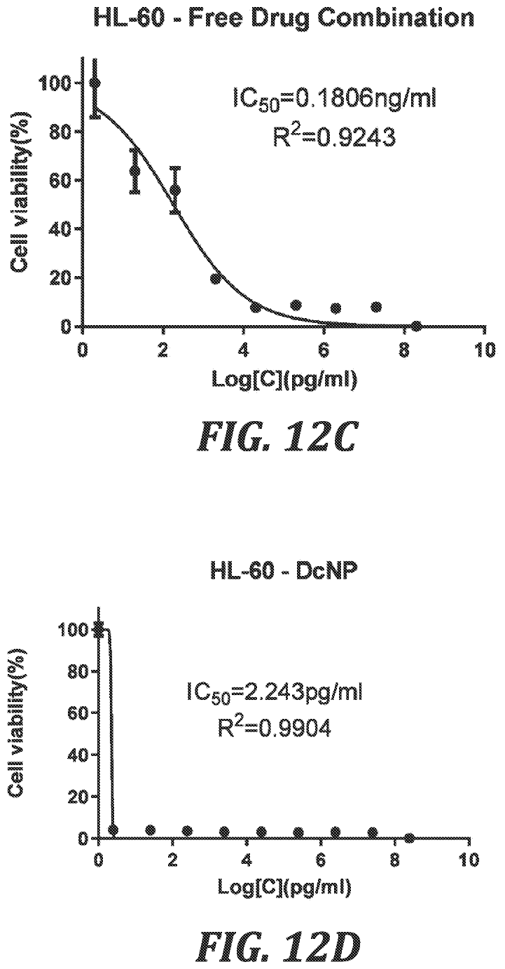

Cell lines were incubated with drug or drug combination for five days, at which point their relative growth was measured using AlamarBlue and analyzed with Prism software. HL-60 cells (FIGS. 12A-12D) demonstrated the highest sensitivity to both free venetoclax alone (IC50: 1.92 ng/mL) and to the free drug combination (IC50: 0.181 ng/mL). K-562 and MOLT-4 were less sensitive to venetoclax: 15.9 μg/mL and 1.96 μg/mL, respectively. K-562 and MOLT-4 cells were also less sensitive to the free drug 1:1 combination: 8.0 and 2.0 μg/mL, respectively. All cells showed similar sensitivities to zanubrutinib: HL-60: 10.3 ug/mL, K-562: 8.3 μg/mL, and MOLT-4: 4.0 μg/mL. HL-60 cells were also assayed against a range of DcNP's that contained drug at equivalent concentrations to the free drug combination assay. The IC50 of HL-60 cells to the DcNP's was found to be 2.2 pg/mL.

The effects of DcNP formulation on the effectiveness of ABT-199 and BGB-3111 to inhibit cells that express Bcl-2 and BTK targets is shown in Table 6. Three immortalized human cell lines were selected based on their expression of Bcl-2 (B-cell lymphoma 2; target of venetoclax) and BTK (Bruton's tyrosine kinase; target of zanubrutinib). Cell lines were seeded in 96-well plates at 75,000 cells per well. In separate sets of wells, venetoclax and zanubrutinib were incubated with the cells for 5 days individually as free drug, together as free drug, and together as the drug combination nanoformulation. An AlamarBlue assay was performed to determine relative inhibition of cell growth due to drug presence.

TABLE 6
Effects of DcNP formulation on the effectiveness of ABT-199 and
BGB-3111 to inhibit cells that express Bcl-2 and BTK targets.

| | Cell line | HL60 | K562 | Molt-4 |
|---|---|---|---|---|
| | Bcl-2 expression | + | − | + |
| | BTK expression | + | + | − |
| Inhibitory | ABT-199 | 1.9 × 10³ | 1.6 × 10⁷ | 2.0 × 10⁶ |
| effects | BGB-3111 | 1.0 × 10⁷ | 8.4 × 10⁶ | 4.0 × 10⁶ |
| (EC₅₀, pg/ml) | A + B free drug | 180.6 | 8.0 × 10⁶ | 1.9 × 10⁶ |
| | A + B DcNPs | 2.2 | ND | ND |

In Vitro Uptake of Free Drug Vs. DcNP-Bound Drug into Leukemic Cells

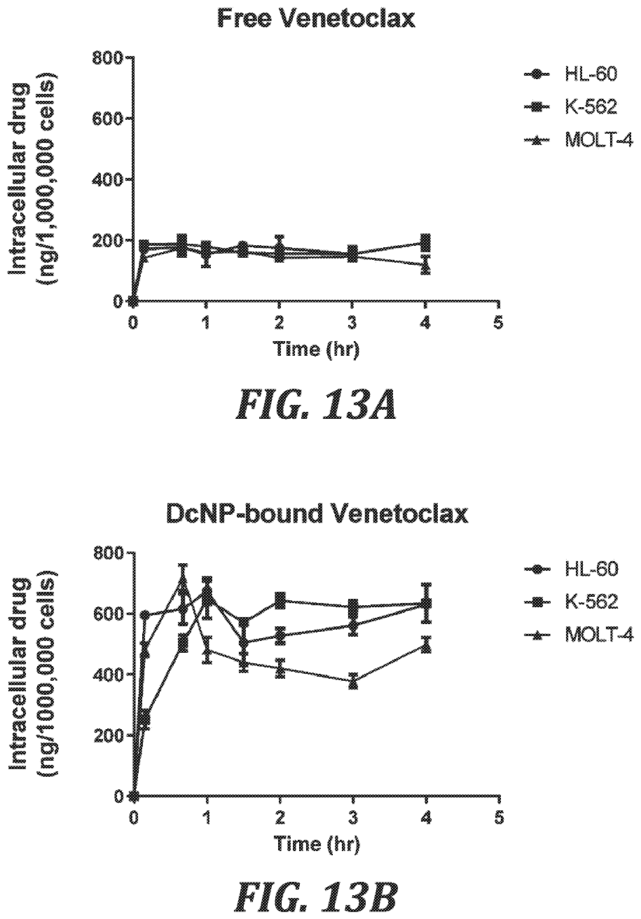

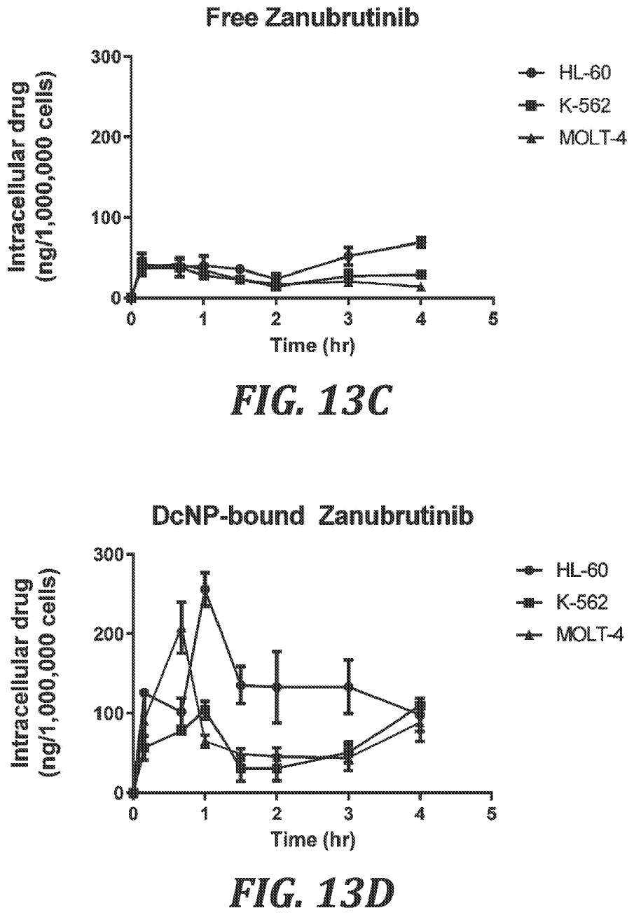

All cell lines incubated with free drug or DcNP's were able to rapidly take up the drug/DcNP's with all cells reaching their peak intracellular drug concentrations within 1 hour of incubation (FIGS. 13A-13D).

Venetoclax reached peak intracellular concentrations at 1 hour, which were maintained until 4 hours (terminal time point). Incubation with free drug reached levels of around 200 ng of drug per million cells (H1-60: 192 ng/million cells; K-562: 192 ng/million cells; MOLT-4: 176 ng/million cells), compared to around 700 ng drug per million cells when incubated with DcNP's (H1-60: 674 ng/million cells; K-562: 647 ng/million cells; MOLT-4: 718 ng/million cells).

Zanubrutinib reached peak intracellular concentrations at 1 hour, which were somewhat maintained until 4 hours (terminal time point). Incubation with free drug reached levels of around 75 ng of drug per million cells (H1-60: 69 ng/million cells; K-562: 109 ng/million cells; MOLT-4: 42 ng/million cells), compared to around 200-650 ng drug per million cells when incubated with DcNP's (H1-60: 256 ng/million cells; K-562: 647 ng/million cells; MOLT-4: 208 ng/million cells).

### In Vivo Pharmacokinetics of DcNP-Bound Venetoclax and Zanubrutinib

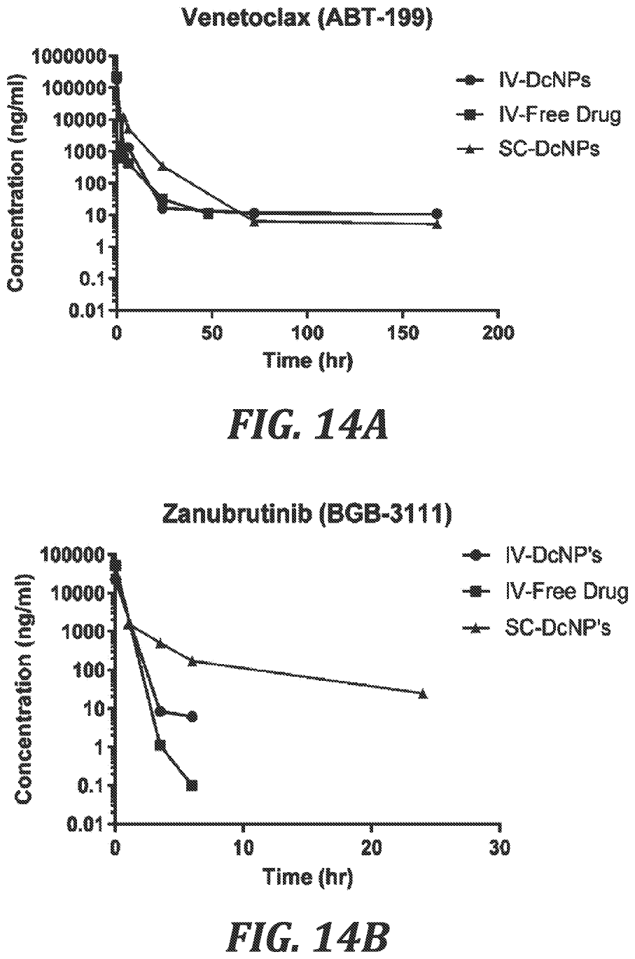

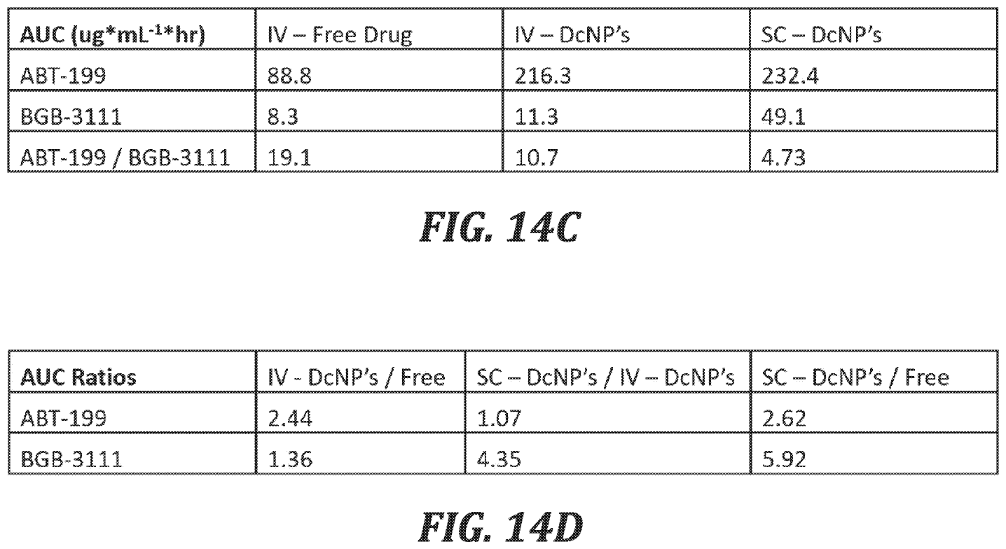

Following administration of the nanoparticles, venetoclax was able to be detected in plasma up to seven days afterwards, while zanubrutinib was detectable less than one day (FIGS. 14A-14D). Of the three groups of mice, the subcutaneous injection group had the highest plasma levels across the seven days of testing. This yielded the highest drug AUC's observed in this study: venetoclax was 232 ug*mL⁻¹*hr (as compared to free drug of 88.8 ug*mL⁻¹*hr) and zanubrutinib was 49 ug*mL⁻¹*hr (as compared to free drug of 8.3 ug*mL⁻¹*hr). Intravenously administered DcNP's had consistently higher AUC's than the free drug injection [venetoclax was 216 ug*mL⁻¹*hr (as compared to free drug of 88.8 ug*mL⁻¹*hr) and zanubrutinib was 11.3 ug*mL⁻¹*hr (as compared to free drug of 8.3 ug*mL⁻¹*hr)], but was not as successful as the subcutaneously delivered DcNP's.

### Discussion

Chronic Lymphocytic Leukemia (CLL) remains challenging to cure due to the cancer's infiltration into sanctuary sites in the body. Small molecule chemotherapy drugs have difficulty reaching and sustaining adequate concentrations for treatment at these sites, including bone marrow and the lymphatic system. In clinical practice, the oral dosage form is considered most desirable for patients due its convenience and ease of administration, though oral delivery is limited by incomplete absorption, fast elimination of drugs from the body, and, subsequently, daily dosing to maintain adequate drug concentrations in the plasma. Daily oral dosing of toxic drugs can also induce many off-target toxicities in patients, most commonly the degradation of the patient's gastrointestinal tract due to oral drug delivery. In addition, most current leukemia treatment consists of a combination of small molecule drugs; monotherapy is usually inadequate due to increased risk of drug resistance by the cancer as well as the large drug dosages imposing a heavy burden on the patient.

The present Example presents a drug combination nanoparticle (DcNP) platform as a suitable vehicle for extended retention and release of small molecule drugs for treating CLL, namely venetoclax (ABT-199) and zanubrutinib (BGB-3111). The platform is stable, scalable, and biocompatible. Venetoclax and zanubrutinib were able to be almost completely incorporated into the DcNP's with little drug loss between the initial and final stages of drug formulation. Percent association of drug to the platform exceeded 90% (FIG. 11). DcNP's were produced primarily at a 10-40 nm size range that remained stable over the course of seventy days (FIG. 10).

When introduced to leukemic cell lines, free drug combinations in vitro of venetoclax and zanubrutinib showed very promising efficacy against the cancer, with IC50's in the low nanogram per milliliter range for HL-60 cells. DcNP's showed even more promising results with an IC50 in the low picogram per milliliter solution range. (FIGS. 12A-12D). The improvement in efficacy of the DcNP's over the free drug combination may be due to enhanced uptake and retention of the DcNP-bound drug as compared to free drug (FIGS. 13A-13D).

Finally, the pharmacokinetics of the subcutaneously injected DcNP's in mice was more favorable than intravenous injection of either free drug or DcNP's (FIGS. 14A-14D). Both drugs were detectable in mouse plasma for a longer period from the subcutaneous route than either intravenous injection. The AUC's of venetoclax and zanubrutinib delivered subcutaneously as DcNP's compared to the intravenously delivered free drug were over 2.62 and 5.92, respectively, demonstrating the ability of DcNP's to deliver drug over a greatly extended period of time compared to free drug.

Drug combination nanoparticles offer a new delivery route for anti-cancer drugs that greatly improves the associated drugs' efficacy against cancer cell growth as well as significantly extending the release and AUC of drug in the body over time, making the DcNP's a superior delivery route of cytotoxic drugs than either the oral or intravenous routes.

By example and without limitation, embodiments are disclosed according to the following enumerated paragraphs:

A1. An injectable aqueous dispersion, comprising:

an aqueous solvent, and

a chemotherapeutic agent composition dispersed in the aqueous solvent to provide the injectable aqueous dispersion, the chemotherapeutic agent composition comprising a combination of chemotherapeutic agents selected from:
gemcitabine and paclitaxel; and
venetoclax and zanubrutinib;

the chemotherapeutic agent composition further comprising one or more compatibilizers comprising a lipid (e.g., a lipid excipient), a lipid conjugate, or a combination thereof;

wherein the chemotherapeutic agents of the chemotherapeutic agent composition exhibit a synergistic chemotherapeutic effect.

A2. The aqueous dispersion of Paragraph A1, wherein the chemotherapeutic agents and the one or more compatibilizers together form an organized composition.

A3. The aqueous dispersion of Paragraph A1 or Paragraph A2, wherein the chemotherapeutic agents and the one or more compatibilizers together comprise a long-range order in the form of a repeating pattern.

A4. The aqueous dispersion of any one of Paragraphs A1 to A3, wherein the chemotherapeutic agents and the one or more compatibilizers together comprise a repetitive multi-drug motif structure.

A5. The aqueous dispersion of any one of Paragraphs A1 to A4, wherein the aqueous dispersion does not comprise a structural feature of a lipid layer, a lipid bilayer, a liposome, or a micelle.

A6. The aqueous dispersion of any one of Paragraphs A1 to A5, wherein the aqueous solvent is selected from a buffered aqueous solvent, saline, and an aqueous solution of 10-100 mM sodium bicarbonate and 0.45 wt % to 0.9 wt % NaCl.

A7. The aqueous dispersion of any one of Paragraphs A1 to A6, wherein the aqueous dispersion comprises each chemotherapeutic agent composition in an amount of 5 wt % or more and 30 wt % or less.

A8. The aqueous dispersion of any one of Paragraphs A1 to A7, wherein the chemotherapeutic agent composition comprises gemcitabine and paclitaxel.

A9. The aqueous dispersion of any one of Paragraphs A1 to A8, wherein the gemcitabine: paclitaxel molar ratio is from about 1:1 to about 50:1.

A10. The aqueous dispersion of any one of Paragraphs A1 to A9, wherein the chemotherapeutic agent composition comprises an AUC of from 1,000 μg·min/mL to 60,000 μg min/mL for gemcitabine and an AUC of from 150 μg·min/mL to 1,000 μg·min/mL for paclitaxel.

A11. The aqueous dispersion of any one of Paragraphs A1 to A10, wherein the paclitaxel exhibits an apparent terminal half-life of from 1.5 h to 5 h.

A12. The aqueous dispersion of any one of Paragraphs A1 to A11, wherein the gemcitabine exhibits an apparent terminal half-life of from 5 h to 20 h.

A13. The aqueous dispersion of any one of Paragraphs A1 to A7, wherein the chemotherapeutic agent composition comprises venetoclax and zanubrutinib.

A14. The aqueous dispersion of any one of Paragraphs A1 to A7 and A13, wherein the venetoclax and zanubrutinib molar ratio is from about 10:1 to about 1:10.

A15. The aqueous dispersion of any one of Paragraphs A1 to A7, A13, and A14, wherein the chemotherapeutic agent composition comprises an AUC of from 150 μg.h/mL to 500 μg.h/mL for venetoclax and an AUC of from 10 μg.h/mL to 100 μg.h/mL for zanubrutinib.

A16. The aqueous dispersion of any one of Paragraphs A1 to A7 and A13 to A15, wherein the venetoclax exhibits an apparent terminal half-life of from 24 h to 75 h.

A17. The aqueous dispersion of any one of Paragraphs A1 to A7 and A13 to A16, wherein the zanubrutinib exhibits an apparent terminal half-life of from 24 h to 80 h.

A18. The aqueous dispersion of any one of Paragraphs A1 to A7 and A13 to A17, comprising a molar ratio of chemotherapeutic agents to the one or more compatibilizers of from about 1:10 to about 1:1.

A19. The aqueous dispersion of any one of Paragraphs A1 to A18, wherein the one or more compatibilizers are selected from 1,2-distearoyl-sn-glycero-3-phosphocholine, 1,2-distearoyl-sn-glycero-3-phosphoethanolamine-N-[poly(ethylene glycol)₂₀₀₀], and a combination thereof.

A20. The aqueous dispersion of any one of Paragraphs A1 to A19, in the form of a suspension.

A21. The aqueous dispersion of any one of Paragraphs A1 to A20, wherein the dispersion remains stable when stored at 25° C. for at least 2 weeks.

A22. A method of treating cancer, comprising:

parenterally administering to a subject in need thereof injectable aqueous dispersion of any one of Paragraphs A1 to A21,

wherein the chemotherapeutic agents of the chemotherapeutic agent composition exhibit a synergistic chemotherapeutic effect.

A23. The method of Paragraph A22, wherein the cancer expresses an upregulation of Bruton tyrosine kinase (BTK), Bcl-2, or both BTK and Bcl-2.

A24. The method of Paragraph A22 or Paragraph A23, wherein the chemotherapeutic agents exhibit a synergistic inhibitory effect on BTK, Bcl-2, or both BTK and Bcl-2.

A25. The method of any one of Paragraph A22 to A24, wherein the chemotherapeutic agent composition comprises gemcitabine and paclitaxel.

A26. The method of any one of Paragraphs A22 to A25, comprising administering a gemcitabine dosage of from 1 mg/kg to 50 mg/kg and a paclitaxel dosage of from 0.1 mg/kg to 50 mg/kg.

A27. The method of any one of Paragraphs A22 to A24, wherein the chemotherapeutic agent composition comprises venetoclax and zanubrutinib.

A28. The method of any one of Paragraphs A22 to A24 and A27, comprising administering a venetoclax dosage of from 0.1 mg/kg to 30 mg/kg and a zanubrutinib dosage of from 0.1 mg/kg to 30 mg/kg.

A29. The method of any one of Paragraphs A22 to A28, wherein the cancer comprises metastatic breast cancer, pancreatic cancer, or a liquid tumor (e.g., leukemia).

A30. The method of any one of Paragraphs A22 to A29, wherein the aqueous dispersion exhibits a 1- to 60-fold higher AUC of each chemotherapeutic agent in mice, when administered subcutaneously, compared to the exposure of each freely solubilized or suspended individual chemotherapeutic agent.

A31. The method of any one of Paragraphs A22 to A30, wherein each chemotherapeutic agent in the combination of chemotherapeutic agents of the aqueous dispersion has a terminal half-life greater than the terminal half-life of each freely solubilized or suspended individual therapeutic agent.

A32. A powder composition comprising a combination of chemotherapeutic agents selected from:
gemcitabine and paclitaxel; and
venetoclax and zanubrutinib; and

the powder composition further comprising one or more compatibilizers comprising a lipid (e.g., a lipid excipient), a lipid conjugate, or a combination thereof;

wherein the chemotherapeutic agents of the combination of chemotherapeutic agents exhibit a synergistic chemotherapeutic effect.

A33. The powder composition of Paragraph A32, wherein the composition comprises a phase transition temperature different from the transition temperature of each individual chemotherapeutic agent when assessed by differential scanning calorimetry.

A34. The powder composition of claim 32 or 33, wherein the composition is in the form of homogeneous distribution of each individual chemotherapeutic agent when viewed by scanning electron microscopy, X-ray diffraction, calorimetry, or any combination thereof.

While illustrative embodiments have been illustrated and described, it will be appreciated that various changes can be made therein without departing from the spirit and scope of the disclosure.

1. An injectable aqueous dispersion, comprising:
an aqueous solvent, and
a chemotherapeutic agent composition dispersed in the aqueous solvent to provide an injectable aqueous dispersion, the chemotherapeutic agent composition comprising a combination of chemotherapeutic agents selected from:
gemcitabine and paclitaxel; and
venetoclax and zanubrutinib; and
one or more compatibilizers comprising a lipid, a lipid conjugate, or a combination thereof;
wherein the chemotherapeutic agents of the chemotherapeutic agent composition exhibit a synergistic and sustained chemotherapeutic effect.

2. The aqueous dispersion of claim 1, wherein the chemotherapeutic agents and the one or more compatibilizers together form an organized composition.

3. The aqueous dispersion of claim 1, wherein the chemotherapeutic agents and the one or more compatibilizers together comprise a long-range physical order in the form of a repeating multiple-drug-domain pattern in an intermediate powder product in producing aqueous dispersion.

4. The aqueous dispersion of claim 1, wherein the chemotherapeutic agents and the one or more compatibilizers together comprise a repetitive multi-drug motif structure.

5. The aqueous dispersion of claim 1, wherein the aqueous dispersion does not comprise a structural feature of a lipid layer, a lipid bilayer, a liposome, or a micelle.

6. The aqueous dispersion of claim 1, wherein the aqueous solvent is selected from a buffered aqueous solvent, saline, and an aqueous solution of 10-100 mM sodium bicarbonate and 0.45 wt % to 0.9 wt % NaCl.

7. The aqueous dispersion of claim 1, wherein the aqueous dispersion comprises each chemotherapeutic agent composition in an amount of 5 wt % or more and 30 wt % or less.

8. (canceled)

9. The aqueous dispersion of claim 1, wherein the gemcitabine: paclitaxel molar ratio is from about 1:1 to about 50:1.

10-13. (canceled)

14. The aqueous dispersion of claim 1, wherein the venetoclax and zanubrutinib molar ratio is from about 10:1 to about 1:10.

15-17. (canceled)

18. The aqueous dispersion of claim 1, comprising a molar ratio of chemotherapeutic agents to the one or more compatibilizers of from about 1:10 to about 1:1.

19. The aqueous dispersion of claim 1, wherein the one or more compatibilizers are selected from 1,2-distearoyl-sn-glycero-3-phosphocholine, 1,2-distearoyl-sn-glycero-3-phosphoethanolamine-N-[poly(ethylene glycol)₂₀₀₀], and a combination thereof.

20. (canceled)

21. The aqueous dispersion of claim 1, wherein the dispersion remains stable when stored at 25° C. for at least 2 weeks.

22. A method of treating cancer, comprising:
parenterally administering to a subject in need thereof the injectable aqueous dispersion of claim 1,
wherein the chemotherapeutic agents of the chemotherapeutic agent composition exhibit a synergistic and sustained chemotherapeutic effect.

23. The method of claim 22, wherein the cancer expresses an upregulation of Bruton tyrosine kinase (BTK), Bcl-2, or both BTK and Bcl-2.

24-25. (canceled)

26. The method of claim 22, comprising administering a gemcitabine dosage of from 1 mg/kg to 50 mg/kg and a paclitaxel dosage of from 0.1 mg/kg to 50 mg/kg.

27. (canceled)

28. The method of claim 22, comprising administering a venetoclax dosage of from 0.1 mg/kg to 30 mg/kg and a zanubrutinib dosage of from 0.1 mg/kg to 30 mg/kg.

29. The method of claim 22, wherein the cancer comprises metastatic breast cancer, pancreatic cancer, or a liquid tumor.

30. (canceled)

31. The method of claim 22, wherein each chemotherapeutic agent in the combination of chemotherapeutic agents of the aqueous dispersion has a terminal half-life greater than the terminal half-life of each freely solubilized or suspended individual therapeutic agent.

32. A powder composition comprising a combination of chemotherapeutic agents selected from:
gemcitabine and paclitaxel; and
venetoclax and zanubrutinib; and
one or more compatibilizers comprising a lipid, a lipid conjugate, or a combination thereof;
wherein the chemotherapeutic agents of the combination of chemotherapeutic agents exhibit a synergistic and sustained chemotherapeutic effect.

33. The powder composition of claim 32, wherein the composition comprises a phase transition temperature different from the transition temperature of each individual chemotherapeutic agent when assessed by differential scanning calorimetry.

34. (canceled)
